(19) United States
(12) Patent Application Publication
(10) Pub. No.: US 2024/0027444 A1
(43) Pub. Date: Jan. 25, 2024

(54) BIOMARKERS FOR PREDICTING IMMUNOGENICITY AND THERAPEUTIC RESPONSES TO ADALIMUMAB IN RHEUMATOID ARTHRITIS PATIENTS

(71) Applicants: Sengenics Corporation Pte Ltd, Singapore (SG); CHINA MEDICAL UNIVERSITY HOSPITAL, Taichung City (TW)

(72) Inventors: Jonathan Michael BLACKBURN, Singapore (SG); Arif ANWAR, Kuala Lumpur (MY); Der-Yuan CHEN, Taichung City (TW); Po-Ku CHEN, Taichung City (TW)

(73) Assignees: Sengenics Corporation Pte Ltd, Singapore (SG); CHINA MEDICAL UNIVERSITY HOSPITAL, Taichung City (TW)

(21) Appl. No.: 18/037,592

(22) PCT Filed: Nov. 11, 2021
(86) PCT No.: PCT/SG2021/050690

§ 371 Date: May 18, 2023

(30) Foreign Application Priority Data
Nov. 18, 2020 (SG) 10202011473Y

(51) Int. Cl. G01N 33/564

(52) U.S. Cl. CPC G01N 33/564; G01N 2800/102; G01N 2800/52

(57) ABSTRACT

A method for predicting an immunogenic and/or therapeutic response to adalimumab 5 from a sample extracted from a rheumatoid arthritis patient by testing the sample for the presence of biomarkers, the biomarkers being autoantibodies to antigens comprising SSB, TROVE2 and ZHX2.

### FIELD OF INVENTION

The invention relates to the detection of immunological biomarkers, particularly autoantibodies, to predict immunogenicity and therapeutic responses to adalimumab in patients with Rheumatoid Arthritis (RA).

### BACKGROUND

Rheumatoid arthritis (RA), a chronic inflammatory articular disease, is characterized by persistent synovitis, cartilage degradation, and bone erosions [1], and tumor necrosis factor (TNF)-α is a crucial inflammatory mediator in RA-related synovitis and joint damage [2]. The importance of the role of TNF-α in RA pathogenesis is supported by the effectiveness of biologics targeting this cytokine [2-4], although the efficacy diminishes in some patients over time (secondary failure) [5]. Accumulating evidence indicates that the presence of anti-drug antibodies (ADAb) in certain patients may be associated with low or undetectable drug levels and ensuing reduction of therapeutic responsiveness to TNF-α inhibitors [6-10]. Such ADAb responses reflect the differential immunogenicity of the given biologic drug triggered in individual patients, which results in some patients developing a neutralising antibody response against the biologic drug and others not. In the face of such uncertainty about whether individual RA patients will show therapeutic responsiveness to TNF-α inhibitors or not [11], physicians hoping to optimize personalized and precision therapy are thus eager to find biomarkers which can predict the emergence of ADAb and the effectiveness of anti-TNF-α biologics.

Proteomics research has been increasingly applied to the identification of novel biomarkers that might be useful for monitoring therapeutic response in RA patients on specific treatments [12-14]. However, there is currently limited knowledge about circulating biomarkers that are able to predict the development of ADAb in RA patients receiving anti-TNF-α therapy.

Autoantibody biomarkers as described herein are autoantibodies to antigens, autoantibodies being antibodies which are produced by an individual which are directed against one or more of the individual's own proteins (‘self’ antigens).

The aim of the present invention therefore is to provide a novel panel of autoantibody biomarkers that are able to predict immunogenicity of adalimumab and therapeutic responses to adalimumab in individual RA patients, prior to treatment with adalimumab, a widely used TNF-α inhibitor marketed under the brand name HUMIRA® and commonly used for the treatment of autoimmune diseases, such as RA, Crohn's Disease and Psoriasis.

### SUMMARY OF INVENTION

In one aspect of the invention, there is provided a method for predicting a response to adalimumab from a sample extracted from a rheumatoid arthritis patient prior to treating the patient with adalimumab, said response being classified as a good response corresponding to anti-drug antibody negative or a poor response corresponding to anti-drug antibody positive, comprising the steps of:
(i) testing the sample for the presence of autoantibody biomarkers; and
(ii) determining whether the patient will develop a good response or a bad response to treatment with adalimumab, based on the detection of said autoantibody biomarkers;
   characterised in that the autoantibody biomarkers are autoantibodies to antigens comprising SSB, TROVE2 and ZHX2, wherein ZHX2 is associated with the good response, and SSB and TROVE2 are associated with the poor response.

Advantageously the autoantibody biomarkers can be used to predict immunogenicity of adalimumab and therapeutic responses to adalimumab in individual rheumatoid arthritis (RA) patients at baseline (i.e. prior to treating the patient with adalimumab).

In one embodiment the sample is tested using a panel of antigens that correspond to the autoantibody biomarkers. Typically the antigens are biotinylated proteins. Advantageously the biotinylation ensures that the antigens are folded in their correct form to ensure accuracy of detection by the autoantibody biomarkers.

In one embodiment the antigens may include one or more additional antigens from the group comprising of PPARD, SPANXN2, HNRNPA2B, TRIB2, CEP55, SH3GL1, FN3K, PANK3, HPCAL1, THRA, AIFM1, ODC1, RPS6KA4, EEF1D, KLF10, EPHA2, PRKAR1A and EAPP.

It should be noted that not all human antigens generate an autoantibody response and it is not possible to predict a priori which human antigens will do so in a given patient cohort—of the 1622 antigens tested, only autoantibodies against the 21 antigens described above are suitable as biomarkers in predicting immunogenicity of adalimumab and therapeutic responses to adalimumab in RA patient at baseline.

In one embodiment each biotinylated protein is formed from a Biotin Carboxyl Carrier Protein (BCCP) folding marker which is fused in-frame with the protein.

In one embodiment the biotinylated proteins are bound to a streptavidin-coated substrate.

Advantageously full-length proteins are expressed as fusions to the BCCP folding marker which itself becomes biotinylated in vivo when the fusion partner is correctly folded. By comparison misfolded fusion partners cause the BCCP to remain in the ‘apo’ (i.e. non-biotinylated) form such that it cannot attach to a streptavidin substrate. Thus only correctly folded fusion proteins become attached to the streptavidin substrate via the biotin moiety appended to the BCCP tag.

In one embodiment the substrate comprises a glass slide, biochip, strip, slide, bead, microtitre plate well, surface plasmon resonance support, microfluidic device, thin film polymer base layer, hydrogel-forming polymer base layer, or any other device or technology suitable for detection of antibody-antigen binding.

In one embodiment the substrate is exposed to a sample extracted from a person, such that autoantibody biomarkers from the sample may bind to the antigens. Typically the sample comprises any or any combination of exosomes, blood, serum, plasma, urine, saliva, amniotic fluid, cerebrospinal fluid, breast milk, semen or bile.

Typically the sample is collected at baseline prior to administration of the first dose of adalimumab.

In one embodiment following exposure to the sample, the substrate is exposed to a fluorescently-tagged secondary antibody to allow the amount of any autoantibodies from the sample bound to the antigens on the panel to be determined. Typically the secondary antibody is anti-human IgG, but it will be appreciated that other secondary antibodies could be used, such as anti-IgM, anti-IgG1, anti-IgG2, anti-IgG3, anti-IgG4 or anti-IgA.

In one embodiment the patient's response to treatment with adalimumab (i.e. the immunogenic and/or therapeutic response outcome to adalimumab in RA patient at baseline) corresponds to the relative or absolute amount of autoantibodies from the baseline sample specifically binding to the antigens.

In one embodiment the method is performed in vitro.

In a further aspect of the invention, there is provided a method for manufacturing a kit for predicting a response to adalimumab from a sample extracted from a rheumatoid arthritis patient prior to treating the patient with adalimumab, comprising the steps of:
for each antigen in a panel, cloning a biotin carboxyl carrier protein folding marker in-frame with a gene encoding the antigen and expressing the resulting biotinylated antigen;
binding the biotinylated antigens to addressable locations on one or more streptavidin-coated substrates, thereby forming an antigen array;
such that the amount of autoantibodies from the sample binding to the antigens on the panel can be determined by exposing the substrate to the sample and measuring the immunogenicity and response;
characterised in that the antigens comprise SSB, TROVE2 and ZHX2.

In one embodiment the antigens further comprise one or more of PPARD, SPANXN2, HNRNPA2B, TRIB2, CEP55, SH3GL1, FN3K, PANK3, HPCAL1, THRA, AIFM1, ODC1, RPS6KA4, EEF1D, KLF10, EPHA2, PRKARIA and EAPP.

In a further aspect of the invention there is provided a method for predicting immunogenicity of adalimumab and therapeutic responses to adalimumab in RA patients at baseline by exposing a composition comprising a panel of antigens as herein described to a sample extracted from a person, and determining the level of autoantibodies from the sample binding to the antigens.

In a yet further aspect of the invention there is provided a method for predicting immunogenicity of adalimumab and therapeutic responses to adalimumab in RA patients at baseline by exposing a composition comprising a panel of antigens as herein described to a sample extracted from a person in vitro, and determining the level of autoantibodies from the sample binding to the antigens.

In further aspect of the invention, there is provided a composition comprising a panel of antigens for predicting an immunogenic and/or therapeutic response to adalimumab in a rheumatoid arthritis patient who has not previously been treated with adalimumab, characterised in that the antigens comprise SSB, TROVE2 and ZHX2.

In one embodiment the antigens further comprise one or more of PPARD, SPANXN2, HNRNPA2B, TRIB2, CEP55, SH3GL1, FN3K, PANK3, HPCAL1, THRA, AIFM1, ODC1, RPS6KA4, EEF1D, KLF10, EPHA2, PRKARIA and EAPP.

In one embodiment the antigens are biotinylated proteins

In one embodiment the amount of one or more autoantibody biomarkers binding in vitro to the antigens in a sample from a patient can be measured to determine the immunogenicity and therapeutic response outcome to adalimumab in an RA patient at baseline.

In yet further aspect of the invention, there is provided a composition comprising a panel of autoantibody biomarkers for predicting an immunogenic and/or therapeutic response to adalimumab in a rheumatoid arthritis patient who has not previously been treated with adalimumab, wherein the level of the autoantibody biomarkers are measured in a sample collected from the patient;
characterised in that the autoantibody biomarkers are specific to antigens comprising SSB, TROVE2 and ZHX2.

### BRIEF DESCRIPTION OF DRAWINGS

It will be convenient to further describe the present invention with respect to the accompanying drawings that illustrate possible arrangements of the invention. Other arrangements of the invention are possible, and consequently the particularity of the accompanying drawings is not to be understood as superseding the generality of the preceding description of the invention.

FIG. 1 illustrates the structure of the E. coli Biotin Carboxyl Carrier Protein domain.

FIG. 2 illustrates the pPRO9 plasmid used as a vector.

FIG. 3 illustrates the distribution of the normalised RFU (i.e. autoantibody responses) for all 21 biomarkers between ADAb-positive (Group A) and ADAb-negative (Group B) RA patients collected at baseline.

FIG. 4 illustrates the discriminatory performance represented as a receiver operating curve (ROC) with an area under the curve (AUC) of 0.835 for all 21 biomarkers (SSB, TROVE2, ZHX2, PPARD, SPANXN2, HNRNPA2B, TRIB2, CEP55, SH3GL1, FN3K, PANK3, HPCAL1, THRA, AIFM1, ODC1, RPS6KA4, EEF1D, KLF10, EPHA2, PRKARIA and EAPP) between ADAb-positive (Group A) and ADAb-negative (Group B) RA patients collected at baseline.

FIG. 5 illustrates the variable importance measure for each of the 21 biomarkers identified in the study.

FIG. 6 illustrates the discriminatory performance represented as a receiver operating curve (ROC) with an area under the curve (AUC) of 0.761 for the core biomarkers (SSB, TROVE2 and ZHX2) between ADAb-positive (Group A) and ADAb-negative (Group B) RA patients collected at baseline.

### DETAILED DESCRIPTION

The invention utilises the Biotin Carboxyl Carrier Protein (BCCP) folding marker which is cloned in-frame with the gene encoding the protein of interest, as described above and in EP1470229. The structure of the E. coli BCCP domain is illustrated in FIG. 1, wherein residues 77-156 are drawn (coordinate file 1bdo) showing the N- and C-termini and the single biotin moiety that is attached to lysine-122 in vivo by biotin ligase.

BCCP acts not only as a protein folding marker but also as a protein solubility enhancer. BCCP can be fused to either the N- or C-terminal of a protein of interest. Full-length proteins are expressed as fusions to the BCCP folding marker which becomes biotinylated in vivo, but only when the protein is correctly folded. Conversely, misfolded proteins drive the misfolding of BCCP such that it is unable to become biotinylated by host biotin ligases. Hence, misfolded proteins are unable to specifically attach to a streptavidin-coated solid support. Therefore only correctly folded proteins become attached to a solid support via the BCCP tag.

The surface chemistry of the support is designed carefully and may use a three-dimensional thin film hydrogel layer (polyethylene glycol; PEG), which retains protein spot morphologies and ensures consistent spot sizes across the array. The PEG layer inhibits non-specific macromolecule absorption, therefore reducing the high background observed using other platforms. The solid support used to immobilize the selected biomarkers thus provides excellent signal-to-noise ratios and low limits of detection (translating in to improved sensitivity). In addition the PEG hydrogel layer also aids preservation of the folded structure and functionality of arrayed proteins and protein complexes post-immobilisation.

Retention of the correct folded structure of immobilised antigens during antibody binding assays (‘immuno-assays’) is particularly advantageous because human antibodies are known in general to specifically recognise and bind to discontinuous, solvent-accessible epitopes on protein surfaces, yet are also known to bind non-specifically to exposed hydrophobic surfaces on unfolded proteins. Thus serological assays carried out on arrays of unfolded proteins typically give rise to many false positive results due to such non-specific binding events (which have no biological relevance), whilst at the same time also giving rise to many false negative results due to the absence of biologically-relevant discontinuous epitopes. By contrast, serological assays carried out on arrays of folded antigens result in detection of biologically meaningful antibody-antigen interactions that are not obscured by high rates of non-specific binding.

As biotinylated proteins bound to a streptavidin-coated surface show negligible dissociation, this interaction therefore provides a superior means for tethering proteins to a planar surface in a controlled orientation and is thus ideal for applications such as protein arrays, SPR and bead-based assays. The use of a compact, folded, biotinylated, 80 residue domain BCCP affords two significant advantages over for example the AviTag and intein-based tag. First, the BCCP domain is cross-recognised by eukaryotic biotin ligases enabling it to be biotinylated efficiently in yeast, insect, and mammalian cells without the need to co-express the E. coli biotin ligase. Second, the N- and C-termini of BCCP are physically separated from the site of biotinylation by 50 Å (as shown in FIG. 1), so the BCCP domain can be thought of as a stalk which presents the recombinant proteins away from the solid support surface, thus minimising any deleterious effects due to immobilisation.

The addition of BCCP permits the monitoring of fusion protein folding by measuring the extent of in vivo biotinylation. This can be measured by standard blotting procedures, using SDS-PAGE or in situ colony lysis and transfer of samples to a membrane, followed by detection of biotinylated proteins using a streptavidin conjugate such as streptavidin-horseradish peroxidase. Additionally, the fact that the BCCP domain is biotinylated in vivo is particularly useful when multiplexing protein purification for fabrication of protein arrays since the proteins can be simultaneously purified from cellular lysates and immobilised in a single step via the high affinity and specificity exhibited by a streptavidin surface.

### Example 1

Materials and Methods

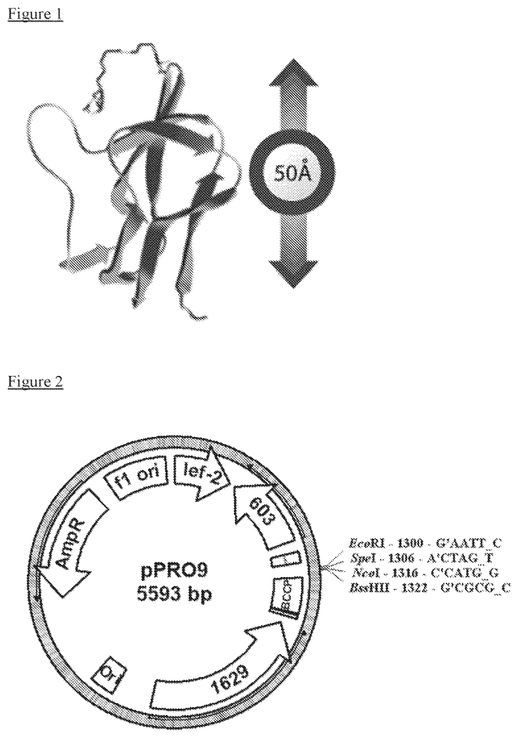

Gene synthesis and cloning. The pPRO9 plasmid (see FIG. 2 below) was constructed by standard techniques and consists of genetic elements encoding a c-myc tag and a BCCP protein domain, preceded by a multiple-cloning site. Synthetic genes encoding individual human antigens were assembled from synthetic oligonucleotides and were cloned into pPRO9 using SpeI and NcoI cloning sites such that each resultant clonal ‘transfer vector’ encoded an in-frame fusion protein comprising a specific human antigen fused to the BCCP tag. The plasmid DNA was purified from transformed bacteria and verified by DNA sequencing. The required sequence congruence within the synthetic gene region was 100%.

Recombinant baculovirus was generated via co-transfection of Sf9 cells (a clonal isolate derived from the parental Spodoptera frugiperda cell line IPLB-Sf-21-AE) with a replication-deficient bacmid vector carrying the viral polyhedrin promoter and a transfer vector carrying a specific coding sequence for a specific antigen. Homologous recombination between the transfer vector and the bacmid within Sf9 cells resulted in formation of a replication competent baculoviral vector encoding the specific antigen fused to the BCCP tag. Successful homologous recombination between the transfer vector and the bacmid within Sf9 cells caused the transfected cells to show signs of viral cytopathic effect (CPE) within few days of culture incubation. The most common CPE observed was the significantly enlargement of average cell size, a consequences of viral progeny propagation. These baculoviruses known as P0 were then released into the culture medium, and viral amplification were done to generate a higher titre of P1 viruses.

Protein Expression. Expression of recombinant antigens was carried out in 24 well blocks using 3 ml cultures containing 6×10⁶ Sf9 cells per well. High titre, low passage, viral stocks of recombinant baculovirus (>107 pfu/ml) were used to infect Sf9 insect cells. The infected cells were then cultured for 72 hours to allow them to produce the recombinant protein of interest. The cells were washed with PBS, resuspended in buffer, and were frozen in aliquots at −80° C. ready for lysis as required. Depending on the transfer vector construct and the nature of the antigen itself, the resultant recombinant protein lysate can be recovered either from the cultured cell or the culture medium. Expression of recombinant proteins was confirmed by SDS-PAGE as well as by Western blot using streptavidin-HRP-based detection. In total, 1622 human antigens were cloned and expressed using this methodology.

Array fabrication. Hydrogel coated, streptavidin-derivatised slides were custom manufactured by Schott and used as substrates on to which the biotinylated proteins were then printed. A total of 9 nanoliters of crude protein lysate was printed on a HS slide in quadruplicate using non-contact piezo printing technology. Print buffer that have a pH between 7.0 and 7.5 were used. The slides were dried by centrifugation (200×g for 5 min) before starting the washing and blocking. The printed arrays were blocked with solutions containing BSA or casein (concentration: 0.1 mg/ml) in a phosphate buffer. The pH was adjusted to be between 7.0 and 7.5 and cold solutions were used (4° C.-20° C.). Slides were not allowed to dry between washes, and were protected from light. In total, each resultant ‘Immunome array’ comprised 1622 antigens, each printed in quadruplicate.

### Experimental Procedure

1. Study Cohort

The study cohort comprised of a total of 62 plasma and serum samples collected from RA patients at baseline (i.e. prior to treatment administration);
i. Run 1: 6 ADAb-positive (“poor response”) and 6 ADAb-negative (“good response”)
ii. Run 2: 24 ADAb-positive (“poor response”) and 26 ADAb-negative (“good response”)

Patients were administered with adalimumab at a dose of 40 mg every other week. The immunogenicity and therapeutic response to adalimumab were evaluated at week 24, the latter by using the EULAR response criteria [15]. EULAR responders were defined as RA patients with good and moderate (“good response”) or poor (“poor response”) EULAR therapeutic responses.

2. Sample Collection and Storage

Peripheral blood samples were collected immediately before the first adalimumab administration (the baseline sample) and also at week 24. After centrifugation at 1000 g for 10 min within 15 min of withdrawal, serum and plasma samples were stored at −70° C.

3. Sample Preparation and Dilution

For each run, samples were placed in a shaking incubator set at 20° C. to allow thawing for 30 minutes. When completely thawed, each sample was vortexed vigorously three times and debris was pelleted by centrifugation for 3 minutes at 13,000 rpm. 11.25 μL of the sample was pipetted into 4.5 mL of Serum Assay Buffer (SAB) containing 0.1% v/v Triton, 0.1% w/v BSA in PBS (20° C.) and vortexed to mix three times. The tube was tilted during aspiration to ensure that the sera was sampled from below the lipid layer at the top but did not touch the bottom of the tube in case of presence of any sediment. Batch records were marked accordingly to ensure that the correct samples were added to the correct tubes. Samples were then randomised prior to assay.

4. Biomarker Assay

Each Immunome array was removed from the storage buffer using forceps, placed in the slide box and rack containing 200 mL cold SAB and shaken on an orbital shaker at 50 rpm, for 5 minutes. After washing, each slide was scanned using a barcode scanner and then placed array side up in an individual slide hybridization chamber containing an individual diluted sera (Step 3 above). All slides were and incubated on a horizontal shaker at 50 rpm for 2 hours at 20° C.

5. Array Washing after Serum Binding

Each Immunome array slide was rinsed twice in individual “Pap jars” with 30 mL SAB, followed by 200 mL of SAB buffer in the slide staining box for 20 minutes on the shaker at 50 rpm at room temperature. All slides were transferred sequentially and in the same orientation.

6. Incubation with Cy3-Anti-Human IgQ

Binding of autoantibodies to the arrayed antigens on the arrays was detected by incubation with Cy3-rabbit anti-human IgG (Dako Cytomation) labelled according to the manufacturer's recommended protocols (GE Healthcare). Arrays were immersed in hybridization solution containing a mixture of Cy3-rabbit anti-human IgG solution diluted 1:1000 in SAB buffer for 2 hours at 50 rpm in 20° C.

7. Washing after Incubation with Cy3-Anti-Human IgG

After incubation, the slide was dipped in 200 mL of SAB buffer, 3 times for 5 minutes at 50 rpm at room temperature. Excess buffer was removed by immersing the slide in 200 mL of pure water for a few minutes. Slides were then dried for 2 min by centrifugation at 240 g at room temperature. Slides were then stored at room temperature until scanning. Fluorescent hybridization signals were measured with excitation at 550 nm and emission at 570 nm using a microarray laser scanner (Agilent) at 10 μm resolution.

Bioinformatic analysis.

1. Image Analysis: Raw Data Extraction

The aim of an image analysis is to evaluate the amount of autoantibody present in the serum sample by measuring the median intensities of all the pixels within each probed spot. A raw .tiff format image file is generated for each slide, i.e. each sample. Automatic extraction and quantification of each spot on the array are performed using the GenePix Pro 7 software (Molecular Devices) which outputs the statistics for each probed spot on the array. This includes the mean and median of the pixel intensities within a spot as well as in its surrounding local background area. A GAL (GenePix Array List) file for the array is generated to enable image analysis. This file contains the information of all probed spots and their positions on the array. Following data extraction, a GenePix Results (.GPR) file is generated for each slide which contains the information for each spot: Protein ID, protein name, foreground intensities, background intensities etc. In the data sheet generated from the experiment, both foreground and background intensities of each spot are represented in relative fluorescence units (RFUs).

2. Data Handling and Pre-Processing

For each slide, antigens and control probes are spotted in quadruplicate on each array. The following steps were performed to verify the quality of the antigen array data before proceeding with data analysis:

Step 1:

Calculate net intensities for each spot by subtracting background signal intensities from the foreground signal intensities of each spot. For each spot, the background signal intensity was calculated using a circular region with three times the diameter of the spot, centered on the spot.

Step 2:

Remove replica spots with net intensity ≤0.

Step 3:

Zero net intensities if only 1 replica spot remaining.

Step 4:

Calculate the coefficient of variant (CV %) for the replica spots on each array.

$$CV \% = \frac{S . D .}{Mean} \times 100 \% \qquad Equation 1$$

Flag any replica spots with only 2 or less replica/s remaining and CV %>20% as “High CV”. The mean net intensity of such replica spots (i.e. antigens) is excluded from downstream analysis.

For antigens/controls with a CV %>20% and with 3 or more replica spots remaining, the replica spots which result in this high CV % value were filtered out. This was done by calculating the standard deviation between the median value of the net intensities and individual net intensities for each set of replica spots. The spot with the highest standard deviation was removed. CV % values were re-calculated and the process repeated.

Step 5:

Calculate the mean of the net intensities for the remaining replica spots.

Step 6:

Inspect signal intensities of two positive controls: IgG and Cy3-BSA.

Step 7:

Carry out a composite normalisation [16] using both quantile-based and total intensity-based modules for each dataset. This method assumes that different samples share a common underlying distribution of their control probes while taking into account the potential existence of flagged spots within them. The Immunome array uses Cy3-labelled biotinylated BSA (Cy3-BSA) replicates as the positive control spots across slides. Hence it is considered as a ‘housekeeping’ probe for normalisation of signal intensities for any given study.

The quantile module adopts the algorithm described by Bolstad et al., 2003 [17]. This reorganisation enables the detection and handling of outliers or flagged spots in any of the Cy3BSA control probes. A total intensity-based module was then implemented to obtain a scaling factor for each sample. This method assumes that post-normalisation, the positive controls should have a common total intensity value across all samples. This composite method aims to normalise the protein array data from variations in their measurements whilst preserving the targeted biological activity across samples. The steps are as follows:

Quantile-Based Normalisation of all cy3BSA across all samples

(i=spot number and j=sample number)
1. Load all Cy3-BSA across all samples, j, into an i×j matrix X
2. Sort spot intensities in each column j of X to get Xₛₒᵣₜ
3. Take the mean across each row i of Xₛₒᵣₜ to get <Xᵢ>

Intensity-Based Normalisation
1. Calculate sum of the mean across each row i, Σ<Xi>
2. For each sample, k, calculate the sum of all Cy3-BSA controls, ΣXk
3. For each sample, k,

$$Scaling factor ( k ) \frac{\sum < X i >}{\sum X k} \qquad Equation 2$$

3. Data Analysis

Batch normalisation: The composite normalised data sets from the assays in the two runs were merged using a ComBat normalisation method [18]. For each protein, this method inputs the net intensity values across all the samples from the 2 data sets and adjusts for any possible batch effects between the two data sets using a parametric empirical Bayes frameworks.

Biomarker Panel Selection: A pipeline was developed which utilises a combination of feature selection and machine learning methodologies to determine the optimal combination of antigens eliciting autoantibody responses from the list of 1622 antigens which are able to provide the best stratification between ADAb-positive and ADAb-negative patients [19]. For feature selection, univariate statistical tests, random forest importance and mutual information metrics were the filter methods used to rank biomarkers.

Biomarker panels were generated by additively selecting the top-ranking biomarkers as inputs to machine learning models up to a total of top 160 biomarkers (top 10% of biomarkers). Any further addition of number of biomarkers did not lead to significant improvements of model performance and would lead to further increase of computational time. To estimate the biomarker panel performance, ROC, sensitivity and specificity was evaluated and the biomarker panel with the best sensitivity and specificity was deemed as the optimal panel to stratify ADAb status. For this analysis, machine learning models were built using Random Forests [20], under default settings with leave-one out cross validation (LOOCV). All analyses were performed using packages available in R. Feature selection was performed using ranger [21] package and all machine learning models were performed using the caret [22] package.

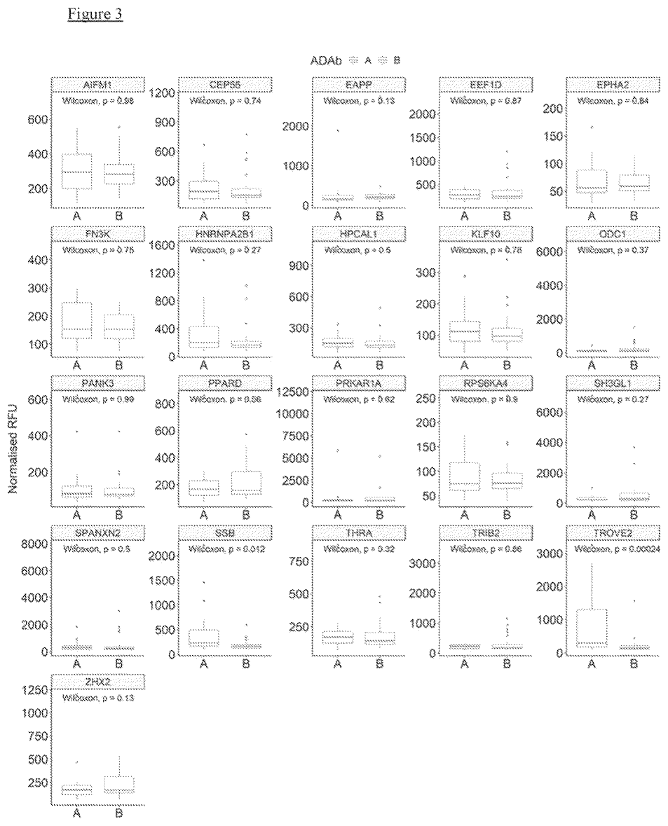

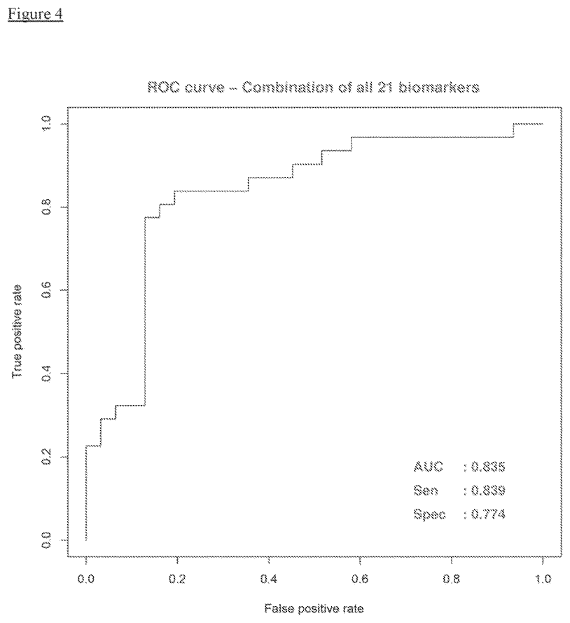

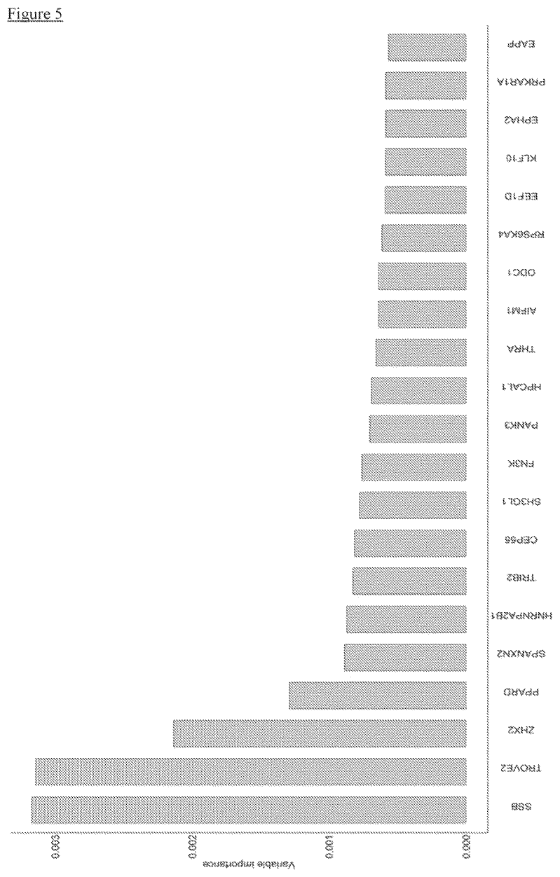

Table 1 shows top 4 best performing biomarker panel from the machine learning models using leave-one out cross validation. The lowest number of antigens with the highest sensitivity and specificity was deemed to be the top biomarker panel. This panel comprises SSB, TROVE2, ZHX2, PPARD, SPANXN2, HNRNPA2B, TRIB2, CEP55, SH3GL1, FN3K, PANK3, HPCAL1, THRA, AIFM1, ODC1, RPS6KA4, EEF1D, KLF10, EPHA2, PRKARIA and EAPP. FIG. 3 shows the distribution of the normalised net intensity (i.e. autoantibody responses) for each of these 21 individual biomarkers in ADAb-positive (Group A) and ADAb-negative (Group B) RA patients at baseline. FIG. 4 shows the discriminatory performance of the combined panel of 21 autoantibody biomarkers represented as a receiver operating curve (ROC), yielding an area under the curve (AUC) of 0.835.

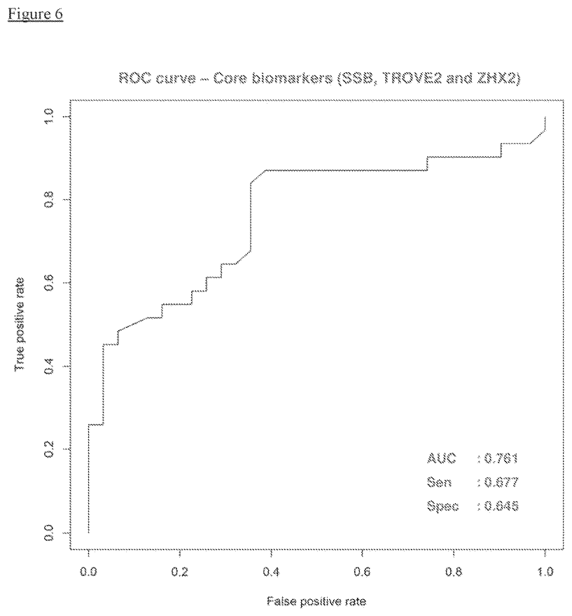

The biomarkers were ranked based on Random Forests estimated variable importance measure [23] derived from each panel (FIG. 5 and Table 2). This further identified a core set of biomarkers which are common across the top 4 biomarker panels, comprising SSB, TROVE2 and ZHX2, with an AUC performance of 0.761 (FIG. 6).

### REFERENCES

1. Choy E H, Panayi G S. Cytokine pathways and joint inflammation in rheumatoid arthritis. N Engl J Med 2001; 344:907-916.
2. Furst D E, Emery P. Rheumatoid arthritis pathophysiology: update on emerging cytokine and cytokine-associated cell targets. Rheumatology (Oxford) 2014; 53:1560-9.
3. Breedveld F C, Weisman M H, Kavanaugh A F, et al. The PREMIER study: a multicenter, randomized, double-blind clinical trial of combination therapy with adalimumab plus methotrexate versus methotrexate alone or adalimumab alone in patients with early, aggressive rheumatoid arthritis who had not had previous methotrexate treatment. Arthritis Rheum 2006; 54:26-37.
4. Kievit W, Fransen J, Adang E M M, et al. Long-term effectiveness and safety of TNF-blocking agents in daily clinical practive: results from the Dutch rheumatoid arthritis monitoring register. Rheumatology (Oxford) 2011; 50:196-203.
5. Bandres Ciga S, Salvatierra J, Lopez-Sidro M, et al. An examination of the mechanisms involved in secondary clinical failure to adalimumab or etanercept in inflammatory arthropathies. J Clin Rheumatol 2015; 21:115-9.
6. Radstake T R, Svenson M, Eijsbouts A M, et al. Formation of antibodies against infliximab and adalimumab strongly correlates with functional drug levels and clinical response in rheumatoid arthritis. Ann Rheum Dis 2009; 68:1739-45.
7. van schouwenburg PA, van de Stadt L A, de Jong R N, et al. Adalimumab elicits a restricted anti-idiotypic antibody response in autoimmune patients resulting in functional neutralization. Ann Rheum Dis 2013; 72:104-109.
8. Bartelds G M, Krieckaert C L, Nurmohamed M T, et al. Development of antidrug antibodies against adalimumab and association with disease activity and treatment failure during long-term follow-up. JAMA 2011; 305:1460-68.
9. Balsa A, Sanmarti R, Rosas J, et al. Drug immunogenicity in patients with inflammatory arthritis and secondary failure to tumor necrosis factor inhibitor therapies: the REASON study. Rheumatology (Oxford) 2018; 57:688-93.
10. Chen D Y, Chen Y M, Tsai W C, et al. Significant associations of antidrug antibody levels with serum drug trough levels and therapeutic response of adalimumab and etanercept treatment in rheumatoid arthritis. Ann Rheum Dis 2015; 74:e16.
11. Smolen J S, Breedveld F C, Burmester G R, et al. Treating rheumatoid arthritis to target: 2014 update of the recommendations of an international task force. Ann Rheum Dis 2016; 75:3-15.
12. Ortea I, Roschitzki B, L6pez-Rodriguez R, et al. Independent candidate serum protein biomarkers of response to adalimumab and to infliximab in rheumatoid arthritis: an exploratory study. PLoS One 2016; 11:e0153140.
13. Cuppen B, Fritsch-Stork R, Eekhout I, et al. Proteomics to predict the response to tumor necrosis factor-alpha inhibitors in rheumatoid arthritis using a supervised cluster-analysis based protein score. Scand J Rheumatol 2018; 47:12-21.
14. Tasaki S, Suzuki K, Kassai Y, et al. Multi-omics monitoring of drug response in rheumatoid arthritis in pursuit of molecular remission. Nat Commun 2018; 9:2755.
15. Van Gestel A M, Prevoo M L, van't Hof M A, et al. Development and validation of the European League Against Rheumatism response criteria for rheumatoid arthritis.

Comparison with the preliminary American College of Rheumatology and the World Health Organization/International League Against Rheumatism Criteria. Arthritis Rheum 1996; 39:34-40.
16. Duarte J, Serufuri J-M, Mulder N, Blackburn J. Protein function microarrays: design, use and bioinformatic analysis in cancer biomarker discovery and quantitation. In: Wang X, editor. Bioinformatics of Human Proteomics. Dordrecht: Springer 2013; 39-74.
17. Bolstad B M, Irizarry R A, Astrand M, Speed T P. A comparison of normalization methods for high density oligonucleotide array data based on bias and variance. Bioinformatics 2003; 19:185-193.
18. Johnson, WE, Rabinovic, A, and Li, C (2007). Adjusting batch effects in microarray expression data using Empirical Bayes methods. Biostatistics 8(1):118-127.
19. Bommert, A., Sun, X., Bischl, B., Rahnenfiuhrer, J., Lang, M., 2020. Benchmark for filter methods for feature selection in high-dimensional classification data. Comput.

Stat. Data Anal. 143, 106839.
20. Breiman, L., 2001. Random forests. Mach. Learn. 45 (1), 5-32.
21. Wright, M. N., Ziegler, A., 2017. Ranger: A fast implementation of random forests for high dimensional data in C++ and R. J. Stat. Softw. 77 (1), 1-17.
22. Kuhn, M. Caret package. Journal of Statistical Software, 2008; 28(5)
Strobl, C., Boulesteix, A.-L., Kneib, T., Augustin, T., Zeileis, A., 2008. Conditional variable importance for random forests. BMC Bioinformatics 9 (307).

TABLE 1

| | No of | | | | |
|---|---|---|---|---|---|
| test | antigen | Biomarkers in panel | ROC | Sens | Spec |
| ranger_permutation | 20 | SSB, TROVE2, ZHX2, PPARD, SPANXN2, HNRNPA2B1, TRIB2, CEP55, | 0.821 | 0.806 | 0.774 |
| | | SH3GL1, FN3K, PANK3, HPCAL1, THRA, AIFM1, ODC1, RPS6KA4, | | | |
| | | EEF1D, KLF10, EPHA2, PRKAR1A | | | |
| ranger_permutation | 21 | SSB, TROVE2, ZHX2, PPARD, SPANXN2, HNRNPA2B1, TRIB2, CEP55, | 0.835 | 0.839 | 0.774 |
| | | SH3GL1, FN3K, PANK3, HPCAL1, THRA, AIFM1, ODC1, RPS6KA4, | | | |
| | | EEF1D, KLF10, EPHA2, PRKAR1A, EAPP | | | |
| ranger_permutation | 28 | SSB, TROVE2, ZHX2, PPARD, SPANXN2, HNRNPA2B1, TRIB2, CEP55, | 0.824 | 0.839 | 0.710 |
| | | SH3GL1, FN3K, PANK3, HPCAL1, THRA, AIFM1, ODC1, RPS6KA4, | | | |
| | | EEF1D, KLF10, EPHA2, PRKAR1A, EAPP, ZNF331, GMPS, POTEE, | | | |
| | | ASPSCR1, ECI2, ETV7, BUD31 | | | |
| ranger_permutation | 29 | SSB, TROVE2, ZHX2, PPARD, SPANXN2, HNRNPA2B1, TRIB2, CEP55, | 0.811 | 0.839 | 0.774 |
| | | SH3GL1, FN3K, PANK3, HPCAL1, THRA, AIFM1, ODC1, RPS6KA4, | | | |
| | | EEF1D, KLF10, EPHA2, PRKAR1A, EAPP, ZNF331, GMPS, POTEE, | | | |
| | | ASPSCR1, ECI2, ETV7, BUD31, ATF3 | | | |

TABLE 2

| test | Importance value | Ranking |
|---|---|---|
| SSB | 0.003172 | 1 |
| TROVE2 | 0.003142 | 2 |
| ZHX2 | 0.002135 | 3 |
| PPARD | 0.001290 | 4 |
| SPANXN2 | 0.000886 | 5 |
| HNRNPA2B1 | 0.000870 | 6 |
| TRIB2 | 0.000825 | 7 |
| CEP55 | 0.000814 | 8 |
| SH3GL1 | 0.000776 | 9 |
| FN3K | 0.000759 | 10 |
| PANK3 | 0.000703 | 11 |
| HPCAL1 | 0.000689 | 12 |
| THRA | 0.000657 | 13 |
| AIFM1 | 0.000639 | 14 |
| ODC1 | 0.000638 | 15 |
| RPS6KA4 | 0.000613 | 16 |
| EEF1D | 0.000590 | 17 |
| KLF10 | 0.000588 | 18 |
| EPHA2 | 0.000587 | 19 |
| PRKAR1A | 0.000587 | 20 |
| EAPP | 0.000565 | 21 |

TABLE 3

| Protein Name | UniprotID | Description |
|---|---|---|
| SSB | P05455 | Lupus La protein |

Nucleotide Sequence (Seq ID No. 1):
>P003196_Q311_Q311_tube_SSB/La_6741_0_NM_
003142.3_0_P05455_0
ATGGCTGAGAACGGCGACAACGAGAAGATGGCTGCTCTCGAGGCT
AAGATCTGCCACCAGATCGAGTACTACTTCGGCGACTTCAACCTG
CCCCGTGACAAGTTCCTGAAGGAACAGATCAAGCTGGACGAGGGC
TGGGTGCCCCTCGAGATCATGATCAAGTTCAACCGTCTGAACCGC
CTGACCACCGACTTCAACGTGATCGTCGAGGCTCTGTCCAAGTCC
AAGGCTGAGCTGATGGAAATCTCCGAGGACAAGACCAAGATCCGT
CGTTCCCCATCCAAGCCCCTGCCCGAAGTGACCGACGAGTACAAG
AACGACGTGAAGAACCGTTCCGTGTACATCAAGGGTTTCCCCACC
GACGCTACCCTGGACGACATCAAGGAATGGCTCGAGGACAAGGGC
CAGGTGCTGAACATCCAGATGCGTCGTACCCTGCACAAGGCTTTC
AAGGGTTCCATCTTCGTGGTGTTCGACTCCATCGAGTCCGCTAAG
AAGTTCGTCGAGACTCCCGGCCAGAAGTACAAGGAAACCGACCTG
CTGATCCTGTTCAAGGACGACTACTTCGCCAAGAAGAACGAGGAA
CGCAAGCAGAACAAGGTCGAGGCCAAGCTGCGTGCTAAGCAAGAG
CAAGAGGCTAAGCAGAAGCTGGAAGAGGACGCTGAGATGAAGTCC
CTGGAAGAGAAGATCGGTTGCCTGCTGAAGTTCTCCGGCGACCTG
GACGACCAGACCTGCCGCGAGGACCTGCACATCCTGTTCTCCAAC
CACGGCGAGATCAAGTGGATCGACTTCGTGCGTGGTGCTAAGGAA
GGCATCATCCTCTTCAAGGAAAAGGCCAAGGAAGCTCTGGGCAAG
GCTAAGGACGCTAACAACGGCAACCTGCAGCTGCGTAACAAGGAA
GTGACCTGGGAGGTCCTCGAGGGCGAGGTCGAGAAGGAAGCCCTG
AAGAAGATCATCGAGGACCAGCAAGAGTCCCTGAACAAGTGGAAG
TCCAAGGGTCGTCGTTTCAAGGGCAAGGGAAAGGGCAACAAGGCT
GCTCAGCCCGGTTCCGGAAAGGGAAAGGTGCAGTTCCAGGGCAAG
AAGACCAAGTTCGCTTCCGACGACGAGCACGATGAGCACGACGAG
AACGGTGCTACCGGTCCCGTGAAGCGTGCTCGTGAAGAGACTGAC
AAGGAAGAACCCGCTTCCAAGCAGCAAAAGACCGAGAACGGCGCT
GGCGACCAG
Protein Sequence (Seq ID No. 22):
>sp|P05455|LA_HUMAN Lupus La protein
OS = Homo sapiens OX = 9606 GN = SSB
PE = 1 SV = 2
MAENGDNEKMAALEAKICHQIEYYFGDFNLPRDKFLKEQIKLDEG
WVPLEIMIKFNRLNRLTTDFNVIVEALSKSKAELMEISEDKTKIR
RSPSKPLPEVTDEYKNDVKNRSVYIKGFPTDATLDDIKEWLEDKG
QVLNIQMRRTLHKAFKGSIFVVFDSIESAKKFVETPGQKYKETDL
LILFKDDYFAKKNEERKQNKVEAKLRAKQEQEAKQKLEEDAEMKS
LEEKIGCLLKFSGDLDDQTCREDLHILFSNHGEIKWIDFVRGAKE
GIILFKEKAKEALGKAKDANNGNLQLRNKEVTWEVLEGEVEKEAL
KKIIEDQQESLNKWKSKGRRFKGKGKGNKAAQPGSGKGKVQFQGK
KTKFASDDEHDEHDENGATGPVKRAREETDKEEPASKQQKTENGA
GDQ

| | | |
|---|---|---|
| TROVE2 | P10155 | HUMAN 60 kDa SS-A/ |
| | | Ro ribonucleoprotein |

Nucleotide Sequence (Seq ID No. 2):
>P001236_CAG_CAGp1_SSA2_6738_Homo sapiens
Sjogren syndrome antigen A2 (60 kDa
ribonucleoprotein
autoantigen SS-A/Ro)_BC036658.2_
AAH36658.1_P10155_0_0_1617_0_1614
ATGGAGGAATCTGTAAACCAAATGCAGCCACTGAATGAGAAGCAG
ATAGCCAATTCTCAGGATGGATATGTATGGCAAGTCACTGACATG
AATCGACTACACCGGTTCTTATGTTTCGGTTCTGAAGGTGGGACT
TATTATATCAAAGAACAGAAGTTGGGCCTTGAAAATGCTGAAGCT
TTAATTAGATTGATTGAAGATGGCAGAGGATGTGAAGTGATACAA
GAAATAAAGTCATTTAGTCAAGAAGGCAGAACCACAAAGCAAGAG
CCTATGCTCTTTGCACTTGCCATTTGTTCCCAGTGCTCCGACATA
AGCACAAAACAAGCAGCATTTAAAGCTGTTTCTGAAGTTTGTCGC
ATTCCTACCCATCTCTTTACTTTTATCCAGTTTAAGAAAGATCTG
AAGGAAAGCATGAAATGTGGCATGTGGGGTCGTGCCCTCCGGAAG
GCTATAGCGGACTGGTACAATGAGAAAGGTGGCATGGCCCTTGCT
CTGGCAGTTACAAAATATAAACAGAGAAATGGCTGGTCTCACAAA
GATCTATTAAGATTGTCACATCTTAAACCTTCCAGTGAAGGACTT
GCAATTGTGACCAAATATATTACAAAGGGCTGGAAAGAAGTTCAT
GAATTGTATAAAGAAAAAGCACTCTCTGTGGAGACTGAAAAATTA
TTAAAGTATCTGGAGGCTGTAGAGAAAGTGAAGCGCACAAGAGAT
GAGCTAGAAGTCATTCATCTAATAGAAGAACATAGATTAGTTAGA
GAACATCTTTTAACAAATCACTTAAAGTCTAAAGAGGTATGGAAG
GCTTTGTTACAAGAAATGCCGCTTACTGCATTACTAAGGAATCTA
GGAAAGATGACTGCTAATTCAGTACTTGAACCAGGAAATTCAGAA
GTATCTTTAGTATGTGAAAAACTGTGTAATGAAAAACTATTAAAA
AAGGCTCGTATACATCCATTTCATATTTTGATCGCATTAGAAACT
TACAAGACAGGTCATGGTCTCAGAGGGAAACTGAAGTGGCGCCCT
GATGAAGAAATTTTGAAAGCATTGGATGCTGCTTTTTATAAAACA
TTTAAGACAGTTGAACCAACTGGAAAACGTTTCTTACTAGCTGTT
GATGTCAGTGCTTCTATGAACCAAAGAGTTTTGGGTAGTATACTC
AACGCTAGTACAGTTGCTGCAGCAATGTGCATGGTTGTCACACGA
ACAGAAAAAGATTCTTATGTAGTTGCTTTTTCCGATGAAATGGTA
CCATGTCCAGTGACTACAGATATGACCTTACAACAGGTTTTAATG
GCTATGAGTCAGATCCCAGCAGGTGGAACTGATTGCTCTCTTCCA
ATGATCTGGGCTCAGAAGACAAACACACCTGCTGATGTCTTCATT
GTATTCACTGATAATGAGACCTTTGCTGGAGGTGTCCATCCTGCT
ATTGCTCTGAGGGAGTATCGAAAGAAAATGGATATTCCAGCTAAA
TTGATTGTTTGTGGAATGACATCAAATGGTTTCACCATTGCAGAC
CCAGATGATAGAGGCATGTTGGATATGTGCGGCTTTGATACTGGA
GCTCTGGATGTAATTCGAAATTTCACATTAGATATGATT
Protein Sequence (Seq ID No. 23):
>sp|P10155|RO60_HUMAN 60 kDa SS-A/Ro
ribonucleoprotein
OS = Homo sapiens OX = 9606 GN = RO60 PE = 1
SV = 2
MEESVNQMQPLNEKQIANSQDGYVWQVTDMNRLHRFLCFGSEGGT
YYIKEQKLGLENAEALIRLIEDGRGCEVIQEIKSFSQEGRTTKQE
PMLFALAICSQCSDISTKQAAFKAVSEVCRIPTHLFTFIQFKKDL
KESMKCGMWGRALRKAIADWYNEKGGMALALAVTKYKQRNGWSHK
DLLRLSHLKPSSEGLAIVTKYITKGWKEVHELYKEKALSVETEKL
LKYLEAVEKVKRTRDELEVIHLIEEHRLVREHLLTNHLKSKEVWK
ALLQEMPLTALLRNLGKMTANSVLEPGNSEVSLVCEKLCNEKLLK
KARIHPFHILIALETYKTGHGLRGKLKWRPDEEILKALDAAFYKT
FKTVEPTGKRFLLAVDVSASMNQRVLGSILNASTVAAAMCMVVTR
TEKDSYVVAFSDEMVPCPVTTDMTLQQVLMAMSQIPAGGTDCSLP
MIWAQKTNTPADVFIVFTDNETFAGGVHPAIALREYRKKMDIPAK
LIVCGMTSNGFTIADPDDRGMLDMCGFDTGALDVIRNFTLDMI

| | | |
|---|---|---|
| ZHX2 | Q9Y6X8 | Zinc fingers and homeoboxes |
| | | protein 2 |

Nucleotide Sequence (Seq ID No. 3):
> P002188_Q305_Q305p3_ZHX2_22882_Homo sapiens
zinc fingers and homeoboxes
2_BC042145.1_Q9Y6X8
ATGGCTAGCAAACGAAAATCTACAACTCCATGCATGGTTCGGACA
TCACAAGTAGTAGAACAAGATGTGCCCGAGGAAGTAGACAGGGCC
AAAGAGAAAGGAATCGGCACACCACAGCCTGACGTGGCCAAGGAC
AGTTGGGCAGCAGAACTTGAAAACTCTTCCAAAGAAAACGAAGTG
ATAGAGGTGAAATCTATGGGGGAAAGCCAGTCCAAAAAACTCCAA
GGTGGTTATGAGTGCAAATACTGCCCCTACTCCACGCAAAACCTG
AACGAGTTCACGGAGCATGTCGACATGCAGCATCCCAACGTGATT
CTCAACCCCCTCTACGTGTGTGCAGAATGTAACTTCACAACCAAA
AAGTACGACTCCCTATCCGACCACAACTCCAAGTTCCATCCCGGG
GAGGCCAACTTCAAGCTGAAGTTAATTAAACGCAATAATCAAACT
GTCTTGGAACAGTCCATCGAAACCACCAACCATGTCGTGTCCATC
ACCACCAGTGGCCCTGGAACTGGTGACAGTGATTCTGGGATCTCG
GTGAGTAAAACCCCCATCATGAAGCCTGGAAAACCAAAAGCGGAT
GCCAAGAAGGTGCCCAAGAAGCCCGAGGAGATCACCCCCGAGAAC
CACGTGGAAGGGACCGCCCGCCTGGTGACAGACACAGCTGAGATC
CTCTCGAGACTCGGCGGGGTGGAGCTCCTCCAAGACACATTAGGA
CACGTCATGCCTTCTGTACAGCTGCCACCAAATATCAACCTTGTG
CCCAAGGTCCCTGTCCCACTAAATACTACCAAATACAACTCTGCC
CTGGATACAAATGCCACGATGATCAACTCTTTCAACAAGTTTCCT
TACCCGACCCAGGCTGAGTTGTCCTGGCTGACAGCTGCCTCCAAA
CACCCAGAGGAGCACATCAGAATCTGGTTTGCCACCCAGCGCTTA
AAGCATGGCATCAGCTGGTCCCCAGAAGAGGTGGAGGAGGCCCGG
AAGAAGATGTTCAACGGCACCATCCAGTCAGTACCCCCGACCATC
ACTGTGCTGCCCGCCCAGTTGGCCCCCACAAAGGTGACGCAGCCC
ATCCTCCAGACGGCTCTACCGTGCCAGATCCTCGGCCAGACTAGC
CTGGTGCTGACTCAGGTGACCAGCGGGTCAACAACCGTCTCTTGC
TCCCCCATCACACTTGCCGTGGCAGGAGTCACCAACCATGGCCAG
AAGAGACCCTTGGTGACTCCCCAAGCTGCCCCCGAACCCAAGCGT
CCACACATCGCTCAGGTGCCAGAGCCCCCACCCAAGGTGGCCAAC
CCCCCGCTCACACCAGCCAGTGACCGCAAGAAGACAAAGGAGCAG
ATAGCACATCTCAAGGCCAGCTTTCTCCAGAGCCAGTTCCCTGAC
GATGCCGAGGTTTACCGGCTCATCGAGGTGACTGGCCTTGCCAGG
AGCGAGATCAAGAAGTGGTTCAGTGACCACCGATATCGGTGTCAA
AGGGGCATCGTCCACATCACCAGCGAATCCCTTGCCAAAGACCAG
TTGGCCATCGCGGCCTCCCGACACGGTCGCACGTATCATGCGTAC
CCAGACTTTGCCCCCCAGAAGTTCAAAGAGAAAACACAGGGTCAG
GTTAAAATCTTGGAAGACAGCTTTTTGAAAAGTTCTTTTCCTACC
CAAGCAGAACTGGATCGGCTAAGGGTGGAGACCAAGCTGAGCAGG
AGAGAGATCGACTCCTGGTTCTCGGAGAGGCGGAAGCTTCGAGAC
AGCATGGAACAAGCTGTCTTGGATTCCATGGGGTCTGGCAAAAAA
GGCCAAGATGTGGGAGCCCCCAATGGTGCTCTGTCTCGACTCGAC
CAGCTCTCCGGTGCCCAGTTAACAAGTTCTCTGCCCAGCCCTTCG
CCAGCAATTGCAAAAAGTCAAGAACAGGTTCATCTCCTGAGGAGC
ACGTTTGCAAGAACCCAGTGGCCTACTCCCCAGGAGTACGACCAG
TTAGCGGCCAAGACTGGCCTGGTCCGAACTGAGATTGTGCGTTGG
TTCAAGGAGAACAGATGCTTGCTGAAAACGGGAACCGTGAAGTGG
ATGGAGCAGTACCAGCACCAGCCCATGGCAGATGATCACGGCTAC
GATGCCGTAGCAAGGAAAGCAACAAAACCCATGGCCGAGAGCCCA
AAGAACGGGGGTGATGTGGTTCCACAATATTACAAGGACCCCAAA
AAGCTCTGCGAAGAGGACTTGGAGAAGTTGGTGACCAGGGTAAAA
GTAGGCAGCGAGCCAGCAAAAGACTGTTTGCCAGCAAAGCCCTCA
GAGGCCACCTCAGACCGGTCAGAGGGCAGCAGCCGGGACGGCCAG
GGTAGCGACGAGAACGAGGAGTCGAGCGTTGTGGATTACGTGGAG
GTGACGGTCGGGGAGGAGGATGCGATCTCAGATAGATCAGATAGC
TGGAGTCAGGCTGCGGCAGAAGGTGTGTCGGAACTGGCTGAATCA
GACTCCGACTGCGTCCCTGCAGAGGCTGGCCAGGCC
Protein Sequence (Seq ID No. 24):
>sp|Q9Y6X8|ZHX2_HUMAN Zinc fingers and
homeoboxes protein 2
OS = Homo sapiens OX = 9606 GN = ZHX2
PE = 1 SV = 1
MASKRKSTTPCMVRTSQVVEQDVPEEVDRAKEKGIGTPQPDVAKD
SWAAELENSSKENEVIEVKSMGESQSKKLQGGYECKYCPYSTQNL
NEFTEHVDMQHPNVILNPLYVCAECNFTTKKYDSLSDHNSKFHPG
EANFKLKLIKRNNQTVLEQSIETTNHVVSITTSGPGTGDSDSGIS
VSKTPIMKPGKPKADAKKVPKKPEEITPENHVEGTARLVTDTAEI
LSRLGGVELLQDTLGHVMPSVQLPPNINLVPKVPVPLNTTKYNSA
LDTNATMINSFNKFPYPTQAELSWLTAASKHPEEHIRIWFATQRL
KHGISWSPEEVEEARKKMFNGTIQSVPPTITVLPAQLAPTKVTQP
ILQTALPCQILGQTSLVLTQVTSGSTTVSCSPITLAVAGVTNHGQ
KRPLVTPQAAPEPKRPHIAQVPEPPPKVANPPLTPASDRKKTKEQ
IAHLKASFLQSQFPDDAEVYRLIEVTGLARSEIKKWFSDHRYRCQ
RGIVHITSESLAKDQLAIAASRHGRTYHAYPDFAPQKFKEKTQGQ
VKILEDSFLKSSFPTQAELDRLRVETKLSRREIDSWFSERRKLRD
SMEQAVLDSMGSGKKGQDVGAPNGALSRLDQLSGAQLTSSLPSPS
PAIAKSQEQVHLLRSTFARTQWPTPQEYDQLAAKTGLVRTEIVRW
FKENRCLLKTGTVKWMEQYQHQPMADDHGYDAVARKATKPMAESP
KNGGDVVPQYYKDPKKLCEEDLEKLVTRVKVGSEPAKDCLPAKPS
EATSDRSEGSSRDGQGSDENEESSVVDYVEVTVGEEDAISDRSDS
WSQAAAEGVSELAESDSDCVPAEAGQA

| | | |
|---|---|---|
| PPARD | Q03181 | Peroxisome proliferator- |
| | | activated receptor delta |

Nucleotide Sequence (Seq ID No. 4):
>P000475_SIG_SIG1-2_PPARD_5467_Homo sapiens
peroxisome proliferative activated receptor
delta transcript variant 2_BC002715.2_
AAH02715.1_Q03181_51381.84_0_1086_0_1083
ATGGAGCAGCCACAGGAGGAAGCCCCTGAGGTCCGGGAAGAGGAG
GAGAAAGAGGAAGTGGCAGAGGCAGAAGGAGCCCCAGAGCTCAAT
GGGGGACCACAGCATGCACTTCCTTCCAGCAGCTACACAGACCTC
TCCCGGAGCTCCTCGCCACCCTCACTGCTGGACCAACTGCAGATG
GGCTGTGACGGGGCCTCATGCGGCAGCCTCAACATGGAGTGCCGG
GTGTGCGGGGACAAGGCATCGGGCTTCCACTACGGTGTTCATGCA
TGTGAGGGGTGCAAGGGCTTCTTCCGTCGTACGATCCGCATGAAG
CTGGAGTACGAGAAGTGTGAGCGCAGCTGCAAGATTCAGAAGAAG
AACCGCAACAAGTGCCAGTACTGCCGCTTCCAGAAGTGCCTGGCA
CTGGGCATGTCACACAACGCTATCCGTTTTGGTCGGATGCCGGAG
GCTGAGAAGAGGAAGCTGGTGGCAGGGCTGACTGCAAATGAGGGG
AGCCAGTACAACCCACAGGTGGCCGACCTGAAGGCCTTCTCCAAG
CACATCTACAATGCCTACCTGAAAAACTTCAACATGACCAAAAAG
AAGGCCCGCAGCATCCTCACCGGCAAAGCCAGCCACACGGCGCCC
TTTGTGATCCACGACATCGAGACATTGTGGCAGGCAGAGAAGGGG
CTGGTGTGGAAGCAGTTGGTGAATGGCCTGCCTCCCTACAAGGAG
ATCAGCGTGCACGTCTTCTACCGCTGCCAGTGCACCACAGTGGAG
ACCGTGCGGGAGCTCACTGAGTTCGCCAAGAGCATCCCCAGCTTC
AGCAGCCTCTTCCTCAACGACCAGGTTACCCTTCTCAAGTATGGC
GTGCACGAGGCCATCTTCGCCATGCTGGCCTCTATCGTCAACAAG
GACGGGCTGCTGGTAGCCAACGGCAGTGGCTTTGTCACCCGTGAG
TTCCTGCGCAGCCTCCGCAAACCCTTCAGTGATATCATTGAGCCT
AAGTTTGAATTTGCTGTCAAGTTCAACGCCCTGGAACTTGATGAC
AGTGACCTGGCCCTATTCATTGCGGCCATCATTCTGTGTGGAGGT
GAG
Protein Sequence (Seq ID No. 25):
>sp|Q03181|PPARD_HUMAN Peroxisome
proliferator-activated receptor delta
OS = Homo sapiens OX = 9606
GN = PPARD PE = 1 SV = 1
MEQPQEEAPEVREEEEKEEVAEAEGAPELNGGPQHALPSSSYTDL
SRSSSPPSLLDQLQMGCDGASCGSLNMECRVCGDKASGFHYGVHA
CEGCKGFFRRTIRMKLEYEKCERSCKIQKKNRNKCQYCRFQKCLA
LGMSHNAIRFGRMPEAEKRKLVAGLTANEGSQYNPQVADLKAFSK
HIYNAYLKNFNMTKKKARSILTGKASHTAPFVIHDIETLWQAEKG
LVWKQLVNGLPPYKEISVHVFYRCQCTTVETVRELTEFAKSIPSF
SSLFLNDQVTLLKYGVHEAIFAMLASIVNKDGLLVANGSGFVTRE
FLRSLRKPFSDIIEPKFEFAVKFNALELDDSDLALFIAAIILCGD
RPGLMNVPRVEAIQDTILRALEFHLQANHPDAQYLFPKLLQKMAD
LRQLVTEHAQMMQRIKKTETETSLHPLLQEIYKDMY

| | | |
|---|---|---|
| SPANXN2 | Q5MJ10 | Sperm protein associated |
| | | with the nucleus on |
| | | the X chromosome N2 |

Nucleotide Sequence (Seq ID No. 5):
>P003098_Q211_Q211_tube_SPANXN2_494119_0_NM_
001009615.1_0_Q5MJ10_0_Insert sequence is gene
optimized by GeneArt_0_0_0
ATGGAACAGCCCACCTCTTCCACCAACGGCGAGAAGCGCAAGTCC
CCCTGCGAGTCCAACAACAAGAAAAACGACGAGATGCAAGAGGCT
CCCAACCGTGTGCTGGCTCCCAAGCAGTCCCTGCAAAAGACCAAG
ACCATCGAGTACCTGACCATCATCGTGTACTACTACCGCAAGCAC
ACCAAGATCAACTCCAACCAGCTCGAGAAGGACCAGTCCCGCGAG
AACTCCATCAACCCCGTGCAAGAGGAAGAGGACGAGGGCCTGGAC
TCCGCTGAGGGATCCTCCCAAGAAGATGAGGACCTGGACAGCTCC
GAGGGTTCCAGCCAAGAGGATGAAGATCTCGACTCCTCCGAGGGC
AGCTCCCAAGAGGACGAGGACTTGGATTCCTCCGAGGGATCTAGT
CAAGAGGACGAGGATCTGGACTCTTCCGAAGGCTCATCTCAAGAA
GATGAAGATTTGGACCCCCCTGAGGGTAGCAGTCAAGAGGATGAG
GACCTCGATTCCAGCGAGGGCTCCTCACAAGAGGGTGGCGAGGAT
Protein Sequence (Seq ID No. 26):
>sp|Q5MJ10|SPXN2_HUMAN Sperm protein associated
with the nucleus on the X chromosome N2
OS = Homo sapiens
OX = 9606 GN = SPANXN2 PE = 1 SV = 1
MEQPTSSTNGEKRKSPCESNNKKNDEMQEAPNRVLAPKQSLQKTK
TIEYLTIIVYYYRKHTKINSNQLEKDQSRENSINPVQEEEDEGLD
SAEGSSQEDEDLDSSEGSSQEDEDLDSSEGSSQEDEDLDSSEGSS
QEDEDLDSSEGSSQEDEDLDPPEGSSQEDEDLDSSEGSSQEGGED

| | | |
|---|---|---|
| HNRNPA2B1 | P22626 | Heterogeneous nuclear |
| | | ribonucleoproteins A2/B1 |

Nucleotide Sequence (Seq ID No. 6):
>P003186_Q311_Q311_tube_HNRNPA2B1_3181_0_
NM_002137.3_0_P22626_0
ATGGAACGCGAGAAAGAGCAGTTCCGCAAGCTGTTCATCGGTGGC
CTGTCCTTCGAGACTACCGAGGAATCCCTGCGCAACTACTACGAG
CAGTGGGGCAAGCTGACCGACTGCGTGGTCATGCGTGACCCCGCT
TCCAAGCGTTCCCGTGGTTTCGGTTTCGTGACCTTCTCCAGCATG
GCTGAGGTGGACGCTGCTATGGCTGCTCGTCCCCACTCCATCGAC
GGTCGTGTGGTCGAGCCTAAGCGTGCTGTGGCTCGTGAAGAGTCC
GGCAAGCCTGGTGCTCACGTGACCGTGAAGAAGCTGTTCGTTGGC
GGTATCAAAGAGGACACCGAGGAACACCACCTGAGGGACTACTTC
GAGGAATACGGCAAGATCGACACCATCGAGATCATCACCGACCGT
CAGTCCGGAAAGAAGCGCGGCTTCGGCTTCGTCACTTTCGACGAC
CACGACCCCGTGGACAAGATCGTGCTGCAGAAGTACCACACCATC
AACGGTCACAACGCTGAAGTGCGCAAGGCTCTGTCCCGTCAAGAG
ATGCAAGAGGTGCAGTCCTCCCGTTCCGGTCGTGGTGGCAACTTC
GGATTCGGCGACTCTCGCGGTGGTGGCGGAAACTTCGGTCCTGGT
CCCGGTTCCAACTTCCGTGGTGGTTCCGACGGTTACGGCTCCGGA
AGAGGTTTCGGCGACGGCTACAACGGCTACGGTGGTGGTCCTGGC
GGTGGAAATTTCGGTGGTTCCCCTGGTTACGGTGGCGGTCGCGGT
GGATACGGCGGAGGTGGTCCAGGATACGGCAACCAGGGTGGCGGT
TACGGCGGTGGTTACGACAACTACGGTGGCGGCAACTACGGTTCC
GGAAACTACAACGACTTCGGCAATTACAACCAGCAGCCCTCCAAC
TACGGCCCCATGAAGTCTGGCAATTTCGGCGGCTCCCGTAACATG
GGTGGTCCTTACGGTGGTGGAAATTACGGTCCCGGTGGTTCCGGT
GGCTCTGGTGGCTACGGCGGTCGTTCCCGTTAC
Protein Sequence (Seq ID No. 27):
>sp|P22626|ROA2_HUMAN Heterogeneous nuclear
ribonucleoproteins A2/B1
OS = Homo sapiens OX = 9606
GN = HNRNPA2B1 PE = 1 SV = 2
MEKTLETVPLERKKREKEQFRKLFIGGLSFETTEESLRNYYEQWG
KLTDCVVMRDPASKRSRGFGFVTFSSMAEVDAAMAARPHSIDGRV
VEPKRAVAREESGKPGAHVTVKKLFVGGIKEDTEEHHLRDYFEEY
GKIDTIEIITDRQSGKKRGFGFVTFDDHDPVDKIVLQKYHTINGH
NAEVRKALSRQEMQEVQSSRSGRGGNFGFGDSRGGGGNFGPGPGS
NFRGGSDGYGSGRGFGDGYNGYGGGPGGGNFGGSPGYGGGRGGYG
GGGPGYGNQGGGYGGGYDNYGGGNYGSGNYNDFGNYNQQPSNYGP
MKSGNFGGSRNMGGPYGGGNYGPGGSGGSGGYGGRSRY

| | | |
|---|---|---|
| TRIB2 | Q92519 | HUMAN Tribbles homolog 2 |

Nucleotide Sequence (Seq ID No. 7):
>P001066_KIN2_KIN2p1_TRB2_28951_Homo sapiens
tribbles homolog 2_BC002637.2_AAH02637.1_
Q92519_0_0_1032_0_1029
ATGAACATACACAGGTCTACCCCCATCACAATAGCGAGATATGGG
AGATCGCGGAACAAAACCCAGGATTTCGAAGAGTTGTCGTCTATA
AGGTCCGCGGAGCCCAGCCAGAGTTTCAGCCCGAACCTCGGCTCC
CCGAGCCCGCCCGAGACTCCGAACTTGTCGCATTGCGTTTCTTGT
ATCGGGAAATACTTATTGTTGGAACCTCTGGAGGGAGACCACGTT
TTTCGTGCCGTGCATCTGCACAGCGGAGAGGAGCTGGTGTGCAAG
GTGTTTGATATCAGCTGCTACCAGGAATCCCTGGCACCGTGCTTT
TGCCTGTCTGCTCATAGTAACATCAACCAAATCACTGAAATTATC
CTGGGTGAGACCAAAGCCTATGTGTTCTTTGAGCGAAGCTATGGG
GACATGCATTCCTTCGTCCGCACCTGCAAGAAGCTGAGAGAGGAG
GAGGCAGCCAGACTGTTCTACCAGATTGCCTCGGCAGTGGCCCAC
TGCCATGACGGGGGGCTGGTGCTGCGGGACCTCAAGCTGCGGAAA
TTCATCTTTAAGGACGAAGAGAGGACTCGGGTCAAGCTGGAAAGC
CTGGAAGACGCCTACATTCTGCGGGGAGATGATGATTCCCTCTCC
GACAAGCATGGCTGCCCGGCTTACGTAAGCCCAGAGATCTTGAAC
ACCAGTGGCAGCTACTCGGGCAAAGCAGCCGACGTGTGGAGCCTG
GGGGTGATGCTGTACACCATGTTGGTGGGGCGGTACCCTTTCCAT
GACATTGAACCCAGCTCCCTCTTCAGCAAGATCCGGCGTGGCCAG
TTCAACATTCCAGAGACTCTGTCGCCCAAGGCCAAGTGCCTCATC
CGAAGCATTCTGCGTCGGGAGCCCTCAGAGCGGCTGACCTCGCAG
GAAATTCTGGACCATCCTTGGTTTTCTACAGATTTTAGCGTCTCG
AATTCAGCATATGGTGCTAAGGAAGTGTCTGACCAGCTGGTGCCG
GACGTCAACATGGAAGAGAACTTGGACCCTTTCTTTAAC
Protein Sequence (Seq ID No. 28):
>sp|Q92519|TRIB2_HUMAN Tribbles homolog 2
OS = Homo sapiens OX = 9606 GN = TRIB2
PE = 1 SV = 1
MNIHRSTPITIARYGRSRNKTQDFEELSSIRSAEPSQSFSPNLGS
PSPPETPNLSHCVSCIGKYLLLEPLEGDHVFRAVHLHSGEELVCK
VFDISCYQESLAPCFCLSAHSNINQITEIILGETKAYVFFERSYG
DMHSFVRTCKKLREEEAARLFYQIASAVAHCHDGGLVLRDLKLRK
FIFKDEERTRVKLESLEDAYILRGDDDSLSDKHGCPAYVSPEILN
TSGSYSGKAADVWSLGVMLYTMLVGRYPFHDIEPSSLFSKIRRGQ
FNIPETLSPKAKCLIRSILRREPSERLTSQEILDHPWFSTDFSVS
NSAYGAKEVSDQLVPDVNMEENLDPFFN

| | | |
|---|---|---|
| CEP55 | Q53EZ4 | Centrosomal protein |
| | | of 55 kDa |

Nucleotide Sequence (Seq ID No. 8):
>P003121_Q211_Q211_tube_CEP55_55165_0_
NM_001127182.1_0_Q53EZ4_0
ATGTCCTCCCGTTCCACCAAGGACCTGATCAAGTCTAAGTGGGGT
TCCAAGCCCTCCAACTCCAAGTCCGAGACTACCCTCGAGAAGCTG
AAGGGCGAGATCGCTCACCTCAAGACCTCCGTGGACGAGATCACC
TCCGGCAAGGGCAAGCTGACCGACAAGGAACGTCACCGTCTGCTC
GAGAAGATCCGTGTGCTCGAGGCTGAGAAGGAAAAGAACGCTTAC
CAGCTGACTGAGAAGGACAAGGAAATCCAGCGTCTGCGCGACCAG
CTGAAGGCTCGTTACTCCACCACCGCTCTGCTGGAACAGCTGGAA
GAAACCACCCGCGAGGGCGAGCGTCGCGAGCAGGTCCTGAAGGCT
CTGTCCGAAGAGAAGGACGTGCTGAAGCAGCAGCTGTCCGCTGCT
ACCTCCCGTATCGCTGAGCTGGAATCCAAGACCAACACCCTGCGT
CTGTCCCAGACCGTGGCTCCCAACTGCTTCAACTCCTCCATCAAC
AACATCCACGAGATGGAAATCCAACTGAAGGACGCTCTCGAGAAG
AACCAGCAGTGGCTGGTGTACGACCAGCAGCGCGAGGTGTACGTG
AAGGGCCTGCTGGCTAAGATCTTCGAGCTGGAAAAGAAGACCGAG
ACTGCTGCTCACTCCCTGCCCCAGCAGACCAAGAAGCCCGAGTCC
GAGGGTTACCTGCAAGAGGAAAAGCAGAAGTGCTACAACGACCTG
CTGGCTTCCGCTAAGAAGGACCTGGAAGTCGAGCGTCAGACCATC
ACCCAGCTGTCCTTCGAGCTGTCCGAGTTCCGTAGGAAGTACGAA
GAGACTCAGAAGGAAGTCCACAACCTGAACCAGCTGCTGTACTCC
CAGCGTCGTGCTGACGTGCAGCACCTCGAGGACGACCGTCACAAG
ACTGAGAAGATCCAGAAGCTGCGCGAAGAGAACGATATCGCTCGT
GGCAAGCTCGAGGAAGAGAAGAAGCGTTCCGAGGAACTGCTGTCC
CAGGTGCAGTTCCTGTACACCTCCCTGCTCAAGCAGCAAGAGGAA
CAGACCCGTGTGGCTCTGTTGGAGCAGCAGATGCAGGCTTGCACC
CTGGACTTCGAGAACGAGAAGCTGGACCGTCAGCACGTCCAGCAC
CAGCTGCACGTGATCCTGAAGGAACTGCGCAAGGCTCGTAACCAG
ATCACCCAGTTGGAGTCCCTGAAGCAGCTGCACGAGTTCGCTATC
ACCGAGCCCCTGGTCACCTTCCAAGGCGAGACTGAGAACCGCGAG
AAGGTGGCCGCTTCCCCCAAGTCCCCCACCGCTGCTCTGAACGAG
TCCCTGGTCGAGTGCCCCAAGTGCAACATCCAGTACCCCGCTACC
GAGCACCGTGACCTGCTGGTGCACGTCGAGTACTGCTCCAAG
Protein Sequence (Seq ID No. 29):
>sp|Q53EZ4|CEP55_HUMAN Centrosomal protein
of 55 kDa
OS = Homo sapiens OX = 9606 GN = CEP55 PE = 1
SV = 3
MSSRSTKDLIKSKWGSKPSNSKSETTLEKLKGEIAHLKTSVDEIT
SGKGKLTDKERHRLLEKIRVLEAEKEKNAYQLTEKDKEIQRLRDQ
LKARYSTTTLLEQLEETTREGERREQVLKALSEEKDVLKQQLSAA
TSRIAELESKTNTLRLSQTVAPNCFNSSINNIHEMEIQLKDALEK
NQQWLVYDQQREVYVKGLLAKIFELEKKTETAAHSLPQQTKKPES
EGYLQEEKQKCYNDLLASAKKDLEVERQTITQLSFELSEFRRKYE
ETQKEVHNLNQLLYSQRRADVQHLEDDRHKTEKIQKLREENDIAR
GKLEEEKKRSEELLSQVQFLYTSLLKQQEEQTRVALLEQQMQACT
LDFENEKLDRQHVQHQLHVILKELRKARNQITQLESLKQLHEFAI
TEPLVTFQGETENREKVAASPKSPTAALNESLVECPKCNIQYPAT
EHRDLLVHVEYCSK

| | | |
|---|---|---|
| SH3GL1 | Q99961 | Endophilin-A2 |

Nucleotide Sequence (Seq ID No. 9):
>P000121_CAN_CAN1-1_SH3GL1_6455_Homo sapiens
SH3-domain GRB2-like
1_BC001270.1_AAH01270.1_Q99961_0_0_1107_0_1104
ATGTCGGTGGCGGGGCTGAAGAAGCAGTTCTACAAGGCGAGCCAG
CTGGTCAGTGAGAAGGTCGGAGGGGCCGAGGGGACCAAGCTGGAT
GATGACTTCAAAGAGATGGAGAAGAAGGTGGATGTCACCAGCAAG
GCGGTGACAGAAGTGCTGGCCAGGACCATCGAGTACCTGCAGCCC
AACCCAGCCTCGCGGGCTAAGCTGACCATGCTCAACACGGTGTCC
AAGATCCGGGGCCAGGTGAAGAACCCCGGCTACCCGCAGTCGGAG
GGGCTTCTGGGCGAGTGCATGATCCGCCACGGGAAGGAGCTGGGC
GGCGAGTCCAACTTTGGTGACGCATTGCTGGATGCCGGCGAGTCC
ATGAAGCGCCTGGCAGAGGTGAAGGACTCCCTGGACATCGAGGTC
AAGCAGAACTTCATTGACCCCCTCCAGAACCTGTGCGAGAAAGAC
CTGAAGGAGATCCAGCACCACCTGAAGAAACTGGAGGGCCGCCGC
CTGGACTTTGACTACAAGAAGAAGCGGCAGGGCAAGATCCCCGAT
GAGGAGCTACGCCAGGCGCTGGAGAAGTTCGAGGAGTCCAAGGAG
GTGGCAGAAACCAGCATGCACAACCTCCTGGAGACTGACATCGAG
CAGGTGAGTCAGCTCTCGGCCCTGGTGGATGCACAGCTGGACTAC
CACCGGCAGGCCGTGCAGATCCTGGACGAGCTGGCGGAGAAGCTC
AAGCGCAGGATGCGGGAAGCTTCCTCACGCCCTAAGCGGGAGTAT
AAGCCGAAGCCCCGGGAGCCCTTTGACCTTGGAGAGCCTGAGCAG
TCCAACGGGGGCTTCCCCTGCACCACAGCCCCCAAGATCGCAGCT
TCATCGTCTTTCCGATCTTCCGACAAGCCCATCCGGACCCCTAGC
CGGAGCATGCCGCCCCTGGACCAGCCGAGCTGCAAGGCGCTGTAC
GACTTCGAGCCCGAGAACGACGGGGAGCTGGGCTTCCATGAGGGC
GACGTCATCACGCTGACCAACCAGATCGATGAGAACTGGTACGAG
GGCATGCTGGACGGCCAGTCGGGCTTCTTCCCGCTCAGCTACGTG
GAGGTGCTTGTGCCCCTGCCGCAG
Protein Sequence (Seq ID No. 30):
>sp|Q99961|SH3G1_HUMAN Endophilin-A2
OS = Homo sapiens OX = 9606 GN = SH3GL1
PE = 1 SV = 1
MSVAGLKKQFYKASQLVSEKVGGAEGTKLDDDFKEMEKKVDVTSK
AVTEVLARTIEYLQPNPASRAKLTMLNTVSKIRGQVKNPGYPQSE
GLLGECMIRHGKELGGESNFGDALLDAGESMKRLAEVKDSLDIEV
KQNFIDPLQNLCEKDLKEIQHHLKKLEGRRLDFDYKKKRQGKIPD
EELRQALEKFEESKEVAETSMHNLLETDIEQVSQLSALVDAQLDY
HRQAVQILDELAEKLKRRMREASSRPKREYKPKPREPFDLGEPEQ
SNGGFPCTTAPKIAASSSFRSSDKPIRTPSRSMPPLDQPSCKALY
DFEPENDGELGFHEGDVITLTNQIDENWYEGMLDGQSGFFPLSYV
EVLVPLPQ

| | | |
|---|---|---|
| FN3K | Q9H479 | Fructosamine-3-kinase |

Nucleotide Sequence (Seq ID No. 10):
>P002359_Q106_Q106p1_FN3K_64122_Homo sapiens
fructosamine 3 kinase_BC042680.1_
AAH42680.1_Q9H479_0_0_930_0_927
ATGGAGCAGCTGCTGCGCGCCGAGCTGCGCACCGCGACCCTGCGG
GCCTTCGGCGGCCCCGGCGCCGGCTGCATCAGCGAGGGCCGAGCC
TACGACACGGACGCAGGCCCAGTGTTCGTCAAAGTCAACCGCAGG
ACGCAGGCCCGGCAGATGTTTGAGGGGGAGGTGGCCAGCCTGGAG
GCCCTCCGGAGCACGGGCCTGGTGCGGGTGCCGAGGCCCATGAAG
GTCATCGACCTGCCGGGAGGTGGGGCCGCCTTTGTGATGGAGCAT
TTGAAGATGAAGAGCTTGAGCAGTCAAGCATCAAAACTTGGAGAG
CAGATGGCAGATTTGCATCTTTACAACCAGAAGCTCAGGGAGAAG
TTGAAGGAGGAGGAGAACACAGTGGGCCGAAGAGGTGAGGGTGCT
GAGCCTCAGTATGTGGACAAGTTCGGCTTCCACACGGTGACGTGC
TGCGGCTTCATCCCGCAGGTGAATGAGTGGCAGGATGACTGGCCG
ACCTTTTTCGCCCGGCACCGGCTCCAGGCGCAGCTGGACCTCATT
GAGAAGGACTATGCTGACCGAGAGGCACGAGAACTCTGGTCCCGG
CTACAGGTGAAGATCCCGGATCTGTTTTGTGGCCTAGAGATTGTC
CCCGCGTTGCTCCACGGGGATCTCTGGTCGGGAAACGTGGCTGAG
GACGACGTGGGGCCCATTATTTACGACCCGGCTTCCTTCTATGGC
CATTCCGAGTTTGAACTGGCAATCGCCTTGATGTTTGGGGGGTTC
CCCAGATCCTTCTTCACCGCCTACCACCGGAAGATCCCCAAGGCT
CCGGGCTTCGACCAGCGGCTGCTGCTCTACCAGCTGTTTAACTAC
CTGAACCACTGGAACCACTTCGGGGGGGAGTACAGGAGCCCTTCG
TTGGGCACCATGCGAAGGCTGCTCAAG
Protein Sequence (Seq ID No. 31):
>sp|Q9H479|FN3K_HUMAN Fructosamine-3-kinase
OS = Homo sapiens OX = 9606 GN = FN3K
PE = 1 SV = 1
MEQLLRAELRTATLRAFGGPGAGCISEGRAYDTDAGPVFVKVNRR
TQARQMFEGEVASLEALRSTGLVRVPRPMKVIDLPGGGAAFVMEH
LKMKSLSSQASKLGEQMADLHLYNQKLREKLKEEENTVGRRGEGA
EPQYVDKFGFHTVTCCGFIPQVNEWQDDWPTFFARHRLQAQLDLI
EKDYADREARELWSRLQVKIPDLFCGLEIVPALLHGDLWSGNVAE
DDVGPIIYDPASFYGHSEFELAIALMFGGFPRSFFTAYHRKIPKA
PGFDQRLLLYQLFNYLNHWNHFGREYRSPSLGTMRRLLK

| | | |
|---|---|---|
| PANK3 | Q9H999 | Pantothenate kinase 3 |

Nucleotide Sequence (Seq ID No. 11):
>P002239_Q106_Q106p2_PANK3_79646_Homo sapiens
pantothenate kinase 3_BC013705.1_AAH13705.1_
Q9H999_0_0_1113_0_1110
ATGAAGATCAAAGATGCCAAGAAACCCTCTTTCCCATGGTTTGGC
ATGGACATTGGGGGAACTCTAGTAAAGCTCTCGTACTTTGAACCT
ATTGATATCACAGCAGAGGAAGAGCAAGAAGAAGTTGAGAGTTTA
AAAAGTATTCGGAAATATTTGACTTCTAACGTGGCATATGGATCC
ACCGGCATTCGGGATGTACACCTTGAACTGAAAGATTTAACACTT
TTTGGCCGAAGAGGGAACTTGCACTTTATCAGGTTTCCAACCCAG
GACCTGCCTACTTTTATCCAAATGGGAAGAGATAAAAACTTCTCA
ACATTGCAGACGGTGCTATGTGCTACAGGAGGTGGTGCTTACAAG
TTTGAAAAAGATTTTCGCACAATTGGAAACCTCCACCTGCACAAA
CTGGATGAACTTGACTGCCTTGTAAAGGGCTTGCTGTATATAGAC
TCTGTCAGTTTCAATGGACAAGCCGAGTGCTATTATTTTGCTAAT
GCCTCAGAACCTGAGCGATGCCAAAAGATGCCTTTTAACCTGGAT
GATCCCTATCCACTGCTTGTAGTGAACATTGGCTCAGGAGTCAGT
ATTTTAGCAGTCCATTCCAAAGACAACTATAAACGAGTGACTGGG
ACAAGCCTTGGAGGGGGTACCTTTCTGGGTTTATGCAGTTTATTG
ACTGGCTGTGAAAGTTTTGAAGAGGCTCTTGAAATGGCATCCAAA
GGTGATAGCACACAAGCTGACAAGCTGGTCCGTGATATTTATGGA
GGAGATTATGAAAGATTTGGTTTGCCAGGTTGGGCTGTAGCATCT
AGTTTTGGGAATATGATTTATAAGGAGAAGCGAGAATCTGTTAGT
AAAGAAGATCTGGCAAGAGCTACTTTAGTTACTATCACCAATAAC
ATTGGTTCTGTGGCACGAATGTGTGCTGTTAATGAGAAAATAAAC
AGAGTTGTCTTTGTTGGAAACTTTTTACGTGTCAATACCCTCTCA
ATGAAACTTTTGGCATATGCACTGGATTACTGGTCAAAAGGTCAA
CTAAAAGCATTGTTTCTAGAACATGAGGGTTACTTTGGAGCAGTT
GGTGCACTTCTTGGGCTGCCAAATTTCAGC
Protein Sequence (Seq ID No. 32):
>sp|Q9H999|PANK3_HUMAN Pantothenate kinase 3
OS = Homo sapiens OX = 9606 GN = PANK3
PE = 1 SV = 1
MKIKDAKKPSFPWFGMDIGGTLVKLSYFEPIDITAEEEQEEVESL
KSIRKYLTSNVAYGSTGIRDVHLELKDLTLFGRRGNLHFIRFPTQ
DLPTFIQMGRDKNFSTLQTVLCATGGGAYKFEKDERTIGNLHLHK
LDELDCLVKGLLYIDSVSFNGQAECYYFANASEPERCQKMPFNLD
DPYPLLVVNIGSGVSILAVHSKDNYKRVTGTSLGGGTFLGLCSLL
TGCESFEEALEMASKGDSTQADKLVRDIYGGDYERFGLPGWAVAS
SFGNMIYKEKRESVSKEDLARATLVTITNNIGSVARMCAVNEKIN
RVVFVGNFLRVNTLSMKLLAYALDYWSKGQLKALFLEHEGYFGAV
GALLGLPNFS

| | | |
|---|---|---|
| HPCAL1 | P37235 | Hippocalcin-like protein 1 |

Nucleotide Sequence (Seq ID No. 12):
>P003172_Q311_Q311_tube_HPCAL1_3241_0_NM_
002149.2_0_P37235_0
ATGGGCAAGCAGAACTCCAAGCTGCGTCCCGAGGTGCTGCAGGAC
CTGCGCGAGAACACCGAGTTCACCGACCACGAGCTGCAAGAGTGG
TACAAGGGTTTCCTGAAGGACTGCCCCACCGGTCACCTGACCGTG
GACGAGTTCAAGAAGATCTACGCTAACTTCTTCCCCTACGGCGAC
GCTTCCAAGTTCGCTGAGCACGTGTTCCGTACCTTCGACACCAAC
GGCGACGGCACCATCGACTTCCGCGAGTTCATCATCGCTCTGTCC
GTGACCTCCCGTGGCAAGCTCGAGCAAAAGCTGAAGTGGGCTTTC
TCGATGTACGACCTGGACGGCAACGGTTACATCTCCCGTTCCGAG
ATGCTCGAGATCGTGCAGGCTATCTACAAGATGGTGTCCTCCGTG
ATGAAGATGCCCGAGGACGAGTCCACCCCCGAGAAGCGTACCGAC
AAGATCTTCCGTCAGATGGACACCAACAACGACGGAAAGCTGTCC
CTGGAAGAGTTCATCCGTGGTGCTAAGTCCGACCCCTCCATCGTG
CGTCTGCTGCAGTGCGACCCATCCTCCGCTTCCCAGTTC
Protein Sequence (Seq ID No. 33):
>sp|P37235|HPCL1_HUMAN Hippocalcin-
like protein 1
OS = Homo sapiens OX = 9606 GN = HPCAL1
PE = 1 SV = 3
MGKQNSKLRPEVLQDLRENTEFTDHELQEWYKGFLKDCPTGHLTV
DEFKKIYANFFPYGDASKFAEHVFRTFDTNGDGTIDFREFIIALS
VTSRGKLEQKLKWAFSMYDLDGNGYISRSEMLEIVQAIYKMVSSV
MKMPEDESTPEKRTDKIFROMDTNNDGKLSLEEFIRGAKSDPSIV
RLLQCDPSSASQF

| | | |
|---|---|---|
| THRA | P10827 | Thyroid hormone |
| | | receptor alpha |

Nucleotide Sequence (Seq ID No. 13):
>P000757_TRN_TRNp2_THRA_7067_Homo sapiens
Homo sapiens thyroid hormone receptor alpha
(erythroblastic leukemia viral (v-erb-a)
onc_BC000261.1_AAH00261.1_P10827_0_0_
1473_0_1470
ATGGAACAGAAGCCAAGCAAGGTGGAGTGTGGGTCAGACCCAGAG
GAGAACAGTGCCAGGTCACCAGATGGAAAGCGAAAAAGAAAGAAC
GGCCAATGTTCCCTGAAAACCAGCATGTCAGGGTATATCCCTAGT
TACCTGGACAAAGACGAGCAGTGTGTCGTGTGTGGGGACAAGGCA
ACTGGTTATCACTACCGCTGTATCACTTGTGAGGGCTGCAAGGGC
TTCTTTCGCCGCACAATCCAGAAGAACCTCCATCCCACCTATTCC
TGCAAATATGACAGCTGCTGTGTCATTGACAAGATCACCCGCAAT
CAGTGCCAGCTGTGCCGCTTCAAGAAGTGCATCGCCGTGGGCATG
GCCATGGACTTGGTTCTAGATGACTCGAAGCGGGTGGCCAAGCGT
AAGCTGATTGAGCAGAACCGGGAGCGGCGGCGGAAGGAGGAGATG
ATCCGATCACTGCAGCAGCGACCAGAGCCCACTCCTGAAGAGTGG
GATCTGATCCACATTGCCACAGAGGCCCATCGCAGCACCAATGCC
CAGGGCAGCCATTGGAAACAGAGGCGGAAATTCCTGCCCGATGAC
ATTGGCCAGTCACCCATTGTCTCCATGCCGGACGGAGACAAGGTG
GACCTGGAAGCCTTCAGCGAGTTTACCAAGATCATCACCCCGGCC
ATCACCCGTGTGGTGGACTTTGCCAAAAAACTGCCCATGTTCTCC
GAGCTGCCTTGCGAAGACCAGATCATCCTCCTGAAGGGGTGCTGC
ATGGAGATCATGTCCCTGCGGGGGCTGTCCGCTACGACCCTGAGA
GCGACACCCTGACGCTGAGTGGGGAGATGGCTGTCAAGCGGGAGC
AGCTCAAGAATGGCGGCCTGGGCGTAGTCTCCGACGCCATCTTTG
AACTGGGCAAGTCACTCTCTGCCTTTAACCTGGATGACACGGAAG
TGGCTCTGCTGCAGGCTGTGCTGCTAATGTCAACAGACCGCTCGG
GCCTGCTGTGTGTGGACAAGATCGAGAAGAGTCAGGAGGCGTACC
TGCTGGCGTTCGAGCACTACGTCAACCACCGCAAACACAACATTC
CGCACTTCTGGCCCAAGCTGCTGATGAAGGAGAGAGAAGTGCAGA
GTTCGATTCTGTACAAGGGGGCAGCGGCAGAAGGCCGGCCGGGGG
GTCACTGGGCGTCCACCCGGAAGGACAGCAGCTTCTCGGAATGCA
TGTTGTTCAGGGTCCGCAGGTCCGGCAGCTTGAGCAGCAGCTTGG
TGAAGCGGGAAGTCTCCAAGGGCCGGTTCTTCAGCACCAGAGCCC
GAAGAGCCCGCAGCAGCGTCTCCTGGAGCTGCTCCACCGAAGCGG
AATTCTCCATGCCCGAGCGGTCTGTGGGGAAGACGACAGCAGTGA
GGCGGACTCCCCGAGCTCCTCTGAGGAGGAACCGGAGGTCTGCGA
GGACCTGGCAGGCAATGCAGCCTCTCCC
Protein Sequence (Seq ID No. 34):
>sp|P10827|THA_HUMAN Thyroid hormone
receptor alpha
OS = Homo sapiens OX = 9606 GN = THRA PE = 1
SV = 1
MEQKPSKVECGSDPEENSARSPDGKRKRKNGQCSLKTSMSGYIPS
YLDKDEQCVVCGDKATGYHYRCITCEGCKGFFRRTIQKNLHPTYS
CKYDSCCVIDKITRNQCQLCRFKKCIAVGMAMDLVLDDSKRVAKR
KLIEQNRERRRKEEMIRSLQQRPEPTPEEWDLIHIATEAHRSTNA
QGSHWKQRRKFLPDDIGQSPIVSMPDGDKVDLEAFSEFTKIITPA
ITRVVDFAKKLPMFSELPCEDQIILLKGCCMEIMSLRAAVRYDPE
SDTLTLSGEMAVKREQLKNGGLGVVSDAIFELGKSLSAFNLDDTE
VALLQAVLLMSTDRSGLLCVDKIEKSQEAYLLAFEHYVNHRKHNI
PHFWPKLLMKEREVQSSILYKGAAAEGRPGGSLGVHPEGQQLLGM
HVVQGPQVRQLEQQLGEAGSLQGPVLQHQSPKSPQQRLLELLHRS
GILHARAVCGEDDSSEADSPSSSEEEPEVCEDLAGNAASP

| | | |
|---|---|---|
| AIFM1 | O95831 | Apoptosis-inducing |
| | | factor 1, mitochondrial |

Nucleotide Sequence (Seq ID No. 14):
>P003305_Q311_Q311_tube_AIFM1_9131_Apoptosis-
inducing factor, mitochondrion-associated,
1 [Homo sapiens]_NM_001130846.2_0_0_0_0_0_0_0
ATGGAAAAGGTCCGCCGCGAGGGTGTCAAGGTCATGCCCAACGCT
ATCGTGCAGTCCGTGGGCGTGTCCTCCGGCAAGCTGCTGATCAAG
CTGAAGGACGGTCGCAAGGTGGAAACCGACCACATCGTGGCTGCT
GTGGGCCTCGAGCCCAACGTCGAGCTGGCTAAGACCGGTGGCCTC
GAGATCGACTCCGACTTCGGTGGTTTCCGTGTGAACGCTGAGCTG
CAGGCTCGTTCCAACATCTGGGTGGCCGGCGACGCTGCTTGCTTC
TACGACATCAAGCTGGGTCGTCGTCGTGTCGAGCACCACGACCAC
GCTGTGGTGTCCGGTCGTCTGGCTGGCGAGAACATGACCGGTGCT
GCTAAGCCCTACTGGCACCAGTCCATGTTCTGGTCCGACCTGGGT
CCCGACGTGGGTTACGAGGCTATCGGCCTGGTGGACTCCTCCCTG
CCCACCGTGGGAGTGTTCGCTAAGGCTACCGCTCAGGACAACCCC
AAGTCCGCTACCGAGCAGTCCGGCACCGGTATCCGTTCCGAGTCC
GAGACTGAGTCCGAGGCTTCCGAGATCACCATCCCCCCCTCCACC
CCCGCTGTGCCTCAAGCTCCTGTGCAGGGCGAGGACTACGGCAAG
GGTGTCATCTTCTACCTGCGTGACAAGGTGGTCGTGGGTATCGTG
CTGTGGAACATCTTCAACCGTATGCCTATCGCCCGCAAGATCATC
AAGGACGGCGAGCAGCACGAGGACCTGAACGAGGTGGCCAAGCTG
TTCAACATCCACGAGGAC
Protein Sequence (Seq ID No. 35):
>sp|O95831|AIFM1_HUMAN Apoptosis-inducing
factor 1, mitochondrial
OS = Homo sapiens OX = 9606
GN = AIFM1 PE = 1 SV = 1
MFRCGGLAAGALKQKLVPLVRTVCVRSPRQRNRLPGNLFQRWHVP
LELQMTRQMASSGASGGKIDNSVLVLIVGLSTVGAGAYAYKTMKE
DEKRYNERISGLGLTPEQKQKKAALSASEGEEVPQDKAPSHVPFL
LIGGGTAAFAAARSIRARDPGARVLIVSEDPELPYMRPPLSKELW
FSDDPNVTKTLRFKQWNGKERSIYFQPPSFYVSAQDLPHIENGGV
AVLTGKKVVQLDVRDNMVKLNDGSQITYEKCLIATGGTPRSLSAI
DRAGAEVKSRTTLFRKIGDFRSLEKISREVKSITIIGGGFLGSEL
ACALGRKARALGTEVIQLFPEKGNMGKILPEYLSNWTMEKVRREG
VKVMPNAIVQSVGVSSGKLLIKLKDGRKVETDHIVAAVGLEPNVE
LAKTGGLEIDSDFGGFRVNAELQARSNIWVAGDAACFYDIKLGRR
RVEHHDHAVVSGRLAGENMTGAAKPYWHQSMFWSDLGPDVGYEAI
GLVDSSLPTVGVFAKATAQDNPKSATEQSGTGIRSESETESEASE
ITIPPSTPAVPQAPVQGEDYGKGVIFYLRDKVVVGIVLWNIFNRM
PIARKIIKDGEQHEDLNEVAKLFNIHED

| | | |
|---|---|---|
| ODC1 | P11926 | Ornithine decarboxylase |

Nucleotide Sequence (Seq ID No. 15):
>P000568_SIG_SIG1-3_ODC1_4953_Homo sapiens
ornithine decarboxylase
1_BC025296.1_AAH25296.1_P11926_62117_0_
1386_0_1383
ATGAACAACTTTGGTAATGAAGAGTTTGACTGCCACTTCCTCGAT
GAAGGTTTTACTGCCAAGGACATTCTGGACCAGAAAATTAATGAA
GTTTCTTCTTCTGATGATAAGGATGCCTTCTATGTGGCAGACCTG
GGAGACATTCTAAAGAAACATCTGAGGTGGTTAAAAGCTCTCCCT
CGTGTCACCCCCTTTTATGCAGTCAAATGTAATGATAGCAAAGCC
ATCGTGAAGACCCTTGCTGCTACCGGGACAGGATTTGACTGTGCT
AGCAAGACTGAAATACAGTTGGTGCAGAGTCTGGGGGTGCCTCCA
GAGAGGATTATCTATGCAAATCCTTGTAAACAAGTATCTCAAATT
AAGTATGCTGCTAATAATGGAGTCCAGATGATGACTTTTGATAGT
GAAGTTGAGTTGATGAAAGTTGCCAGAGCACATCCCAAAGCAAAG
TTGGTTTTGCGGATTGCCACTGATGATTCCAAAGCAGTCTGTCGT
CTCAGTGTGAAATTCGGTGCCACGCTCAGAACCAGCAGGCTCCTT
TTGGAACGGGCGAAAGAGCTAAATATCGATGTTGTTGGTGTCAGC
TTCCATGTAGGAAGCGGCTGTACCGATCCTGAGACCTTCGTGCAG
GCAATCTCTGATGCCCGCTGTGTTTTTGACATGGGGGCTGAGGTT
GGTTTCAGCATGTATCTGCTTGATATTGGCGGTGGCTTTCCTGGA
TCTGAGGATGTGAAACTTAAATTTGAAGAGATCACCGGCGTAATC
AACCCAGCGTTGGACAAATACTTTCCGTCAGACTCTGGAGTGAGA
ATCATAGCTGAGCCCGGCAGATACTATGTTGCATCAGCTTTCACG
CTTGCAGTTAATATCATTGCCAAGAAAATTGTATTAAAGGAACAG
ACGGGCTCTGATGACGAAGATGAGTCGAGTGAGCAGACCTTTATG
TATTATGTGAATGATGGCGTCTATGGATCATTTAATTGCATACTC
TATGACCACGCACATGTAAAGCCCCTTCTGCAAAAGAGACCTAAA
CCAGATGAGAAGTATTATTCATCCAGCATATGGGGACCAACATGT
GATGGCCTCGATCGGATTGTTGAGCGCTGTGACCTGCCTGAAATG
CATGTGGGTGATTGGATGCTCTTTGAAAACATGGGCGCTTACACT
GTTGCTGCTGCCTCTACGTTCAATGGCTTCCAGAGGCCGACGATC
TACTATGTGATGTCAGGGCCTGCGTGGCAACTCATGCAGCAATTC
CAGAACCCCGACTTCCCACCCGAAGTAGAGGAACAGGATGCCAGC
ACCCTGCCTGTGTCTTGTGCCTGGGAGAGTGGGATGAAACGCCAC
AGAGCAGCCTGTGCTTCGGCTAGTATTAATGTG
Protein Sequence (Seq ID No. 36):
>sp|P11926|DCOR_HUMAN Ornithine decarboxylase
OS = Homo sapiens OX = 9606 GN = ODC1 PE = 1
SV = 2
MNNFGNEEFDCHFLDEGFTAKDILDQKINEVSSSDDKDAFYVADL
GDILKKHLRWLKALPRVTPFYAVKCNDSKAIVKTLAATGTGFDCA
SKTEIQLVQSLGVPPERIIYANPCKQVSQIKYAANNGVQMMTFDS
EVELMKVARAHPKAKLVLRIATDDSKAVCRLSVKFGATLRTSRLL
LERAKELNIDVVGVSFHVGSGCTDPETFVQAISDARCVFDMGAEV
GFSMYLLDIGGGFPGSEDVKLKFEEITGVINPALDKYFPSDSGVR
IIAEPGRYYVASAFTLAVNIIAKKIVLKEQTGSDDEDESSEQTFM
YYVNDGVYGSFNCILYDHAHVKPLLQKRPKPDEKYYSSSIWGPTC
DGLDRIVERCDLPEMHVGDWMLFENMGAYTVAAASTFNGFQRPTI
YYVMSGPAWQLMQQFQNPDFPPEVEEQDASTLPVSCAWESGMKRH
RAACASASINV

| | | |
|---|---|---|
| RPS6KA4 | O75676 | Ribosomal protein S6 |
| | | kinase alpha-4 |

Nucleotide Sequence (Seq ID No. 16):
>P001321_Q305_Q305p4_RPS6KA4_8986_Homo sapiens
RPS6KA4 ribosomal protein S6 kinase, 90 kDa,
polypeptide 4_BC047896_AAH47896_O75676_0_0_
1575_0_1572
ATGGGGGACGAGGACGACGATGAGAGCTGCGCCGTGGAGCTGOGG
ATCACAGAAGCCAACCTGACCGGGCACGAGGAGAAGGTGAGCGTG
GAGAACTTCGAGCTGCTCAAGGTGCTGGGCACGGGAGCCTACGGC
AAGGTGTTCCTGGTGCGGAAGGCGGGCGGGCACGACGCGGGGAAG
CTGTACGCCATGAAGGTGCTGCGCAAGGCGGCGCTGGTGCAGCGC
GCCAAGACGCAGGAGCACACGCGCACCGAGCGCTCGGTGCTGGAG
CTGGTGCGCCAGGCGCCCTTCCTGGTCACGCTGCACTACGCTTTC
CAGACGGATGCCAAGCTGCACCTCATCCTGGACTATGTGAGCGGC
GGGGAGATGTTCACCCACCTCTACCAGCGCCAGTACTTCAAGGAG
GCTGAGGTGCGCGTGTATGGGGGTGAGATCGTGCTGGCCCTGGAA
CACCTGCACAAGCTCGGCATCATTTACCGAGACCTGAAACTGGAG
AATGTGCTGCTGGACTCCGAGGGCCACATTGTCCTCACGGACTTC
GGGCTGAGCAAGGAGTTCCTGACGGAGGAGAAAGAGCGGACCTTC
TCCTTCTGTGGCACCATCGAGTACATGGCCCCCGAAATCATCCGT
AGCAAGACGGGGCATGGCAAGGCTGTGGACTGGTGGAGCCTGGGC
ATCTTGCTCTTCGAGCTGCTGACGGGGGCCTCGCCCTTCACCCTG
GAGGGCGAGAGGAACACGCAGGCTGAGGTGTCTCGACGGATCCTG
AAGTGCTCCCCTCCCTTCCCCCCTCGGATCGGGCCCGTGGCGCAG
GACCTGCTGCAGCGGCTGCTTTGTAAGGATCCTAAGAAGCGATTG
GGCGCGGGGCCCCAGGGGGCACAAGAAGTCCGGAACCATCCCTTC
TTCCAGGGCCTCGATTGGGTGGCTCTGGCTGCCAGGAAGATTCCA
GCCCCATTCCGGCCCCAAATCCGCTCAGAGCTGGATGTGGGCAAC
TTTGCGGAGGAATTCACTCGGCTGGAGCCTGTCTACTCACCCCCT
GGCAGCCCCCCACCTGGGGACCCCCGAATCTTTCAGGGATACTCC
TTTGTGGCACCCTCCATTCTCTTTGACCACAACAACGCGGCCGAG
ATCATGTGCAAAATCCGCGAGGGGCGCTTCTCCCTTGACGGGGAG
GCCTGGCAGGGTGTATCCGAGGAAGCCAAGGAGCTGGTCCGAGGG
CTCCTGACCGTGGACCCCGCCAAGCGGCTGAAGCTCGAGGGACTG
CGGGGCAGCTCGTGGCTGCAGGACGGCAGCGCGCGCTCCTCGCCC
CCGCTCCGGACGCCCGACGTGCTCGAGTCCTCTGGGCCCGCAGTG
CGCTCGGGTCTCAACGCCACCTTCATGGCATTCAACCGGGGCAAG
CGGGAGGGCTTCTTCCTGAAGAGCGTGGAGAACGCACCCCTGGCC
AAGCGGCGGAAGCAGAAGCTGCGGAGCGCCACCGCCTCCCGCCGG
GGCTCCCCTGCACCAGCCAACCCGGGCCGAGCCCCCGTCGCCGCC
AAAGGGGCCCCCCGCCGAGCCAACGGCCCCCTGCCCCCCTCC
Protein Sequence (Seq ID No. 37):
>sp|O75676|KS6A4_HUMAN Ribosomal protein
S6 kinase alpha-4
OS = Homo sapiens OX = 9606 GN = RPS6KA4
PE = 1 SV = 1
MGDEDDDESCAVELRITEANLTGHEEKVSVENFELLKVLGTGAYG
KVFLVRKAGGHDAGKLYAMKVLRKAALVQRAKTQEHTRTERSVLE
LVRQAPFLVTLHYAFQTDAKLHLILDYVSGGEMFTHLYQRQYFKE
AEVRVYGGEIVLALEHLHKLGIIYRDLKLENVLLDSEGHIVLTDF
GLSKEFLTEEKERTFSFCGTIEYMAPEIIRSKTGHGKAVDWWSLG
ILLFELLTGASPFTLEGERNTQAEVSRRILKCSPPFPPRIGPVAQ
DLLQRLLCKDPKKRLGAGPQGAQEVRNHPFFQGLDWVALAARKIP
APFRPQIRSELDVGNFAEEFTRLEPVYSPPGSPPPGDPRIFQGYS
FVAPSILFDHNNAVMTDGLEAPGAGDRPGRAAVARSAMMQDSPFF
QQYELDLREPALGQGSFSVCRRCRQRQSGQEFAVKILSRRLEANT
QREVAALRLCQSHPNVVNLHEVHHDQLHTYLVLELLRGGELLEHI
RKKRHFSESEASQILRSLVSAVSFMHEEAGVVHRDLKPENILYAD
DTPGAPVKIIDFGFARLRPQSPGVPMQTPCFTLQYAAPELLAQQG
YDESCDLWSLGVILYMMLSGQVPFQGASGQGGQSQAAEIMCKIRE
GRFSLDGEAWQGVSEEAKELVRGLLTVDPAKRLKLEGLRGSSWLQ
DGSARSSPPLRTPDVLESSGPAVRSGLNATFMAFNRGKREGFFLK
SVENAPLAKRRKQKLRSATASRRGSPAPANPGRAPVASKGAPRRA
NGPLPPS

| | | |
|---|---|---|
| EEF1D | P29692 | Elongation factor 1-delta |

Nucleotide Sequence (Seq ID No. 17):
>P001467_CAG_CAGp2_EEF1D_1936_Homo sapiens
Homo sapiens eukaryotic translation
elongation factor 1 delta (guanine nucleotide
exchang_BC007847.2_AAH07847.1_P29692_0_0_
1944_0_1941
ATGAGGAGCGGGAAGGCCTCCTGCACCCTGGAGACCGTGTGGGAA
GACAAGCACAAGTATGAGGAGGCCGAGCGGCGCTTCTACGAACAC
GAGGCCACACAGGCGGCCGCCTCCGCCCAGCAGCTGCCAGCCGAG
GGGCCAGCCATGAATGGGCCCGGCCAGGACGACCCTGAGGACGCT
GATGAGGCGGAAGCCCCTGACGGCGGCAGCAGGCGTGATCCCAGG
AAGAGCCAGGACAGCAGGAAGCCCCTGCAGAAAAAGAGGAAGCGC
TCCCCCAAGAGCGGGCTCGGCCCCGCGGACCTGGCCCTCCTGGGC
CTCTCGGCCGAACGTGTGTGGCTGGACAAGTCACTTTTCGACCAG
GCAGAGAGCTCCTACCGCCAGAAGCTGGCAGATGTGGCTGCCCAG
GCAGCCTGGCCTCCTGCCTTGGCCCCTTGGGGTCTCTGCACCCAT
GGAAACCAGGTGGCCTGCCACCACGTGACCTGGGGGATCTGGGTC
AACAAGTCCTCCTTCGACCAGGCTGAGCGGGCCTTCGTGGAGTGG
TCTCAGGCCCTGTTGCTGGCCCCCGAGGGCAGCCGCAGGCAGGGG
ACTCCCAACACAGGCCAGCAGGTGGCCGTCCCCGACCTGGCCCAC
CAGCCCAGCCCACCGGTCAATGGCCAGCCCCCGCTGGGCAGCCTG
CAGGCACTGGTTCGGGAGGTGTGGCTGGAGAAGCCCCGGTATGAT
GCAGCCGAGAGGGGCTTCTACGAGGCCCTGTTTGACGGCCATCCC
CCAGGGAAGGTGCGCCTGCAAGAGCGAGCCGGCCTGGCCGAGGGT
GCCCGGGGGGCCGCAGAGACCGGCGGGGCCGCAACATCTTAGGGA
ACAAGCGGGCCGGGCTGCGACGGGCCGATGGGGAGGCCCCCTCTG
CCTTGCCCTACTGTTACTTCCTGCAGAAGGATGCAGAGGCCCCCT
GGCTCAGCAAGCCTGCCTACGACAGCGCCGAGTGCCGCCACCACG
CTGCCGAGGCCCTGCGTGTGGCCTGGTGCCTCGAAGCTGCCTCCC
TGTCTCACCGACCCGGTCCTCGGTCTGGCCTGTCCGTGTCCAGCC
TGAGACCCAACAGAAAAATGGCTACAAACTTCCTAGCACATGAGA
AGATCTGGTTCGACAAGTTCAAATATGACGACGCAGAAAGGAGAT
TCTACGAGCAGATGAACGGGCCTGTGGCAGGTGCCTCCCGCCAGG
AGAACGGCGCCAGCGTGATCCTCCGTGACATTGCGAGAGCCAGAG
AGAACATCCAGAAATCCCTGGCTGGAAGCTCAGGCCCCGGGGCCT
CCAGCGGCACCAGCGGAGACCACGGTGAGCTCGTCGTCCGGATTG
CCAGTCTGGAAGTGGAGAACCAGAGTCTGCGTGGCGTGGTACAGG
AGCTGCAGCAGGCCATCTCCAAGCTGGAGGCCCGGCTGAACGTGC
TGGAGAAGAGCTCGCCTGGCCACCGGGCCACGGCCCCACAGACCC
AGCACGTATCTCCCATGCGCCAAGTGGAGCCCCCAGCCAAGAAGC
CAGCCACACCAGCAGAGGATGACGAGGATGATGACATTGACCTGT
TTGGCAGTGACAATGAGGAGGAGGACAAGGAGGCGGCACAGCTGC
GGGAGGAGCGGCTACGGCAGTACGCGGAGAAGAAGGCCAAGAAGC
CTGCACTGGTGGCCAAGTCCTCCATCCTGCTGGATGTCAAGCCTT
GGGATGATGAGACGGACATGGCCCAGCTGGAGGCCTGTGTGCGCT
CTATCCAGCTGGACGGGCTGGTCTGGGGGGCTTCCAAGCTGGTGC
CCGTGGGCTACGGTATCCGGAAGCTACAGATTCAGTGTGTGGTGG
AGGACGACAAGGTGGGGACAGACTTGCTGGAGGAGGAGATCACCA
AGTTTGAGGAGCACGTGCAGAGTGTCGATATCGCAGCTTTCAACA
AGATC
Protein Sequence (Seq ID No. 38):
>sp|P29692|EF1D_HUMAN Elongation factor 1-delta
OS = Homo sapiens OX = 9606 GN = EEF1D PE = 1
SV = 5
MATNFLAHEKIWFDKFKYDDAERRFYEQMNGPVAGASRQENGASV
ILRDIARARENIQKSLAGSSGPGASSGTSGDHGELVVRIASLEVE
NQSLRGVVQELQQAISKLEARLNVLEKSSPGHRATAPQTQHVSPM
RQVEPPAKKPATPAEDDEDDDIDLFGSDNEEEDKEAAQLREERLR
QYAEKKAKKPALVAKSSILLDVKPWDDETDMAQLEACVRSIQLDG
LVWGASKLVPVGYGIRKLQIQCVVEDDKVGTDLLEEEITKFEEHV
QSVDIAAFNKI

| | | |
|---|---|---|
| KLF10 | Q13118 | Krueppel-like factor 10 |

Nucleotide Sequence (Seq ID No. 18):
>P000598_TRN_TRNp1_TIEG_7071_Homo sapiens
TGFB inducible early growth
response_BC011538.1_AAH11538.1_Q53QU8_0_0_
1410_0_1407
ATGGAGGAAAGAATGGAAATGATTTCTGAAAGGCCAAAAGAGAGT
ATGTATTCCTGGAACAAAACTGCAGAGAAAAGTGATTTTGAAGCT
GTAGAAGCACTTATGTCAATGAGCTGCAGTTGGAAGTCTGATTTT
AAGAAATACGTTGAAAACAGACCTGTTACACCAGTATCTGATTTG
TCAGAGGAAGAGAATCTGCTTCCGGGAACACCTGATTTTCATACA
ATCCCAGCATTTTGTTTGACTCCACCTTACAGTCCTTCTGACTTT
GAACCCTCTCAAGTGTCAAATCTGATGGCACCAGCGCCATCTACT
GTACACTTCAAGTCACTCTCAGATACTGCCAAACCTCACATTGCC
GCACCTTTCAAAGAGGAAGAAAAGAGCCCAGTATCTGCCCCCAAA
CTCCCCAAAGCTCAGGCAACAAGTGTGATTCGTCATACAGCTGAT
GCCCAGCTATGTAACCACCAGACCTGCCCAATGAAAGCAGCCAGC
ATCCTCAACTATCAGAACAATTCTTTTAGAAGAAGAACCCACCTA
AATGTTGAGGCTGCAAGAAAGAACATACCATGTGCCGCTGTGTCA
CCAAACAGATCCAAATGTGAGAGAAACACAGTGGCAGATGTTGAT
GAGAAAGCAAGTGCTGCACTTTATGACTTTTCTGTGCCTTCCTCA
GAGACGGTCATCTGCAGGTCTCAGCCAGCCCCTGTGTCCCCACAA
CAGAAGTCAGTGTTGGTCTCTCCACCTGCAGTATCTGCAGGGGGA
GTGCCACCTATGCCGGTCATCTGCCAGATGGTTCCCCTTCCTGCC
AACAACCCTGTTGTGACAACAGTCGTTCCCAGCACTCCTCCCAGC
CAGCCACCAGCCGTTTGCCCCCCTGTTGTGTTCATGGGCACACAA
GTCCCCAAAGGCGCTGTCATGTTTGTGGTACCCCAGCCCGTTGTG
CAGAGTTCAAAGCCTCCGGTGGTGAGCCCGAATGGCACCAGACTC
TCTCCCATTGCCCCTGCTCCTGGGTTTTCCCCTTCAGCAGCAAAA
GTCACTCCTCAGATTGATTCATCAAGGATAAGGAGTCACATCTGT
AGCCACCCAGGATGTGGCAAGACATACTTTAAAAGTTCCCATCTG
AAGGCCCACACGAGGACGCACACAGGAGAAAAGCCTTTCAGCTGT
AGCTGGAAAGGTTGTGAAAGGAGGTTTGCCCGTTCTGATGAACTG
TCCAGACACAGGCGAACCCACACGGGTGAGAAGAAATTTGCGTGC
CCCATGTGTGACCGGCGGTTCATGAGGAGTGACCATTIGACCAAG
CATGCCCGGCGCCATCTATCAGCCAAGAAGCTACCAAACTGGCAG
ATGGAAGTGAGCAAGCTAAATGACATTGCTCTACCTCCAACCCCT
GCTCCCACACAG
Protein Sequence (Seq ID No. 39):
>sp|Q13118|KLF10_HUMAN Krueppel-like factor 10
OS = Homo sapiens OX = 9606 GN = KLF10 PE = 1
SV = 1
MLNFGASLQQTAEERMEMISERPKESMYSWNKTAEKSDFEAVEAL
MSMSCSWKSDFKKYVENRPVTPVSDLSEEENLLPGTPDFHTIPAF
CLTPPYSPSDFEPSQVSNLMAPAPSTVHFKSLSDTAKPHIAAPFK
EEEKSPVSAPKLPKAQATSVIRHTADAQLCNHQTCPMKAASILNY
QNNSFRRRTHLNVEAARKNIPCAAVSPNRSKCERNTVADVDEKAS
AALYDFSVPSSETVICRSQPAPVSPQQKSVLVSPPAVSAGGVPPM
PVICQMVPLPANNPVVTTVVPSTPPSQPPAVCPPVVFMGTQVPKG
AVMFVVPQPVVQSSKPPVVSPNGTRLSPIAPAPGFSPSAAKVTPQ
IDSSRIRSHICSHPGCGKTYFKSSHLKAHTRTHTGEKPFSCSWKG
CERRFARSDELSRHRRTHTGEKKFACPMCDRRFMRSDHLTKHARR
HLSAKKLPNWQMEVSKLNDIALPPTPAPTQ

| | | |
|---|---|---|
| EPHA2 | P29317 | Ephrin type-A receptor 2 |

Nucleotide Sequence (Seq ID No. 19):
>P003284_Q311_Q311_tube_EPHA2_1969_0_
NM_004431.3_0_P29317_0
ATGGAACTGCAGGCTGCTCGTGCTTGCTTCGCTCTGCTGTGGGGT
TGCGCTTTGGCTGCTGCAGCTGCTGCTCAGGGCAAAGAGGTGGTC
CTGCTGGACTTCGCTGCTGCCGGTGGCGAACTGGGATGGCTGACT
CACCCTTACGGCAAGGGCTGGGACCTGATGCAGAACATCATGAAC
GACATGCCCATCTACATGTACTCCGTGTGCAACGTGATGTCCGGC
GACCAGGACAACTGGCTGCGTACCAACTGGGTGTACCGTGGCGAG
GCTGAGCGCATCTTCATCGAGCTGAAGTTCACCGTGCGCGACTGC
AACTCCTTCCCTGGTGGTGCTTCCAGCTGCAAAGAGACTTTCAAC
CTGTACTACGCTGAGTCCGACCTGGACTACGGCACCAACTTCCAG
AAGCGTCTGTTCACCAAGATCGACACTATCGCTCCCGACGAGATC
ACCGTGTCCTCCGACTTCGAGGCTCGTCACGTGAAGCTGAACGTC
GAGGAACGCTCCGTGGGTCCCCTGACCCGCAAGGGATTCTACCTG
GCTTTCCAGGACATCGGTGCTTGCGTGGCCCTGCTGTCCGTGCGT
GTGTACTACAAGAAGTGCCCCGAGCTGCTCCAGGGCCTGGCTCAC
TTCCCTGAGACTATCGCTGGTTCCGACGCTCCCTCCCTGGCTACC
GTTGCTGGTACTTGCGTGGACCACGCTGTGGTGCCACCTGGTGGC
GAGGAACCTCGTATGCACTGCGCTGTGGACGGCGAGTGGCTGGTG
CCTATCGGTCAATGCCTGTGCCAGGCTGGTTACGAGAAGGTCGAG
GACGCTTGCCAGGCTTGCTCCCCCGGTTTCTTCAAGTTCGAGGCT
TCCGAGTCCCCCTGCCTGGAATGCCCTGAACACACCCTGCCTTCC
CCAGAGGGTGCTACCTCCTGCGAGTGCGAAGAGGGCTTCTTCCGT
GCTCCCCAGGACCCCGCTTCTATGCCTTGCACCCGTCCTCCCTCC
GCTCCCCACTACCTGACTGCTGTCGGCATGGGTGCTAAGGTCGAG
CTGCGTTGGACCCCCCCTCAGGATTCTGGTGGTCGCGAGGACATC
GTCTACTCCGTGACCTGCGAGCAGTGCTGGCCTGAGTCTGGCGAA
TGCGGTCCCTGCGAGGCTTCTGTGCGCTACTCTGAGCCTCCTCAC
GGCCTGACCCGTACCTCTGTGACCGTGTCCGACCTCGAGCCCCAC
ATGAACTACACCTTCACCGTCGAGGCCCGTAACGGTGTCTCCGGA
CTGGTCACCTCCCGTTCCTTCCGTACCGCTTCCGTGTCCATCAAC
CAGACCGAGCCCCCCAAAGTGCGCCTGGAAGGACGTTCTACCACC
TCCCTGTCCGTGTCTTGGTCCATCCCCCCACCTCAGCAGTCCCGT
GTGTGGAAGTACGAAGTGACCTACCGCAAGAAGGGCGACTCCAAC
TCTTACAACGTGCGTCGTACCGAGGGTTTCAGCGTGACCCTGGAC
GACCTGGCTCCCGACACCACCTACCTGGTGCAAGTGCAGGCTCTG
ACCCAAGAGGGCCAGGGTGCTGGTTCCAAGGTGCACGAGTTCCAG
ACCCTGTCCCCCGAGGGTTCCGGAAACTTGGCTGTGATCGGCGGT
GTCGCTGTGGGTGTCGTGCTGCTGTTGGTGCTGGCTGGTGTCGGC
TTCTTCATCCACCGTCGTCGCAAGAACCAGCGTGCTCGTCAGTCC
CCTGAGGACGTGTACTTCTCCAAGTCCGAGCAGCTGAAGCCCCTC
AAGACCTACGTGGACCCCCACACTTACGAGGACCCCAACCAGGCT
GTGCTCAAGTTCACTACCGAGATCCACCCCTCCTGCGTGACCCGT
CAGAAAGTGATCGGTGCTGGCGAGTTCGGCGAGGTGTACAAGGGA
ATGCTCAAGACTTCCTCCGGCAAGAAAGAGGTGCCCGTCGCTATC
AAGACCCTGAAGGCTGGCTACACCGAGAAGCAGCGTGTGGACTTC
CTGGGAGAGGCTGGTATCATGGGCCAGTTCTCCCACCACAACATC
ATCCGTCTGGAAGGTGTCATCTCCAAGTACAAGCCCATGATGATC
ATTACCGAGTACATGGAAAACGGCGCCCTGGACAAGTTCCTGCGC
GAGAAGGATGGCGAGTTCTCCGTGCTGCAGCTCGTGGGAATGCTG
CGTGGTATCGCTGCTGGCATGAAGTACCTGGCCAACATGAATTAC
GTGCACAGGGACCTGGCTGCTCGCAACATCCTGGTCAACTCCAAC
CTCGTGTGCAAGGTGTCAGACTTCGGCCTGTCCCGCGTGCTCGAG
GACGATCCTGAGGCTACCTACACCACCTCCGGTGGAAAGATCCCC
ATCCGTTGGACCGCTCCCGAGGCTATCTCTTACCGCAAGTTCACC
TCCGCTTCCGACGTGTGGTCCTTCGGTATCGTGATGTGGGAAGTG
ATGACCTACGGCGAGCGCCCCTACTGGGAGCTGTCTAACCACGAA
GTCATGAAGGCTATCAACGACGGTTTCCGTCTGCCCACCCCTATG
GACTGCCCCTCCGCTATCTACCAGCTGATGATGCAATGCTGGCAG
CAAGAGCGTGCTAGGCGTCCCAAGTTCGCTGACATCGTGTCTATC
CTCGACAAGCTGATCCGCGCTCCTGACTCCCTGAAAACCCTGGCT
GACTTCGACCCCCGTGTGTCCATCCGCCTGCCTTCTACCTCTGGC
TCCGAGGGTGTCCCTTTCCGTACTGTGTCCGAGTGGCTCGAGTCC
ATCAAGATGCAGCAGTACACCGAGCACTTCATGGCTGCTGGTTAC
ACCGCTATCGAGAAGGTGGTGCAGATGACCAACGACGACATCAAG
CGTATCGGCGTGCGTCTGCCCGGTCACCAGAAGAGGATCGCTTAC
TCCCTGCTGGGCCTGAAGGACCAAGTGAACACCGTGGGTATCCCC
ATC
Protein Sequence (Seq ID No. 40):
>sp|P29317|EPHA2_HUMAN Ephrin type-A
receptor 2
OS = Homo sapiens OX = 9606 GN = EPHA2 PE = 1
SV = 2
MELQAARACFALLWGCALAAAAAAQGKEVVLLDFAAAGGELGWLT
HPYGKGWDLMQNIMNDMPIYMYSVCNVMSGDQDNWLRTNWVYRGE
AERIFIELKFTVRDCNSFPGGASSCKETFNLYYAESDLDYGTNFQ
KRLFTKIDTIAPDEITVSSDFEARHVKLNVEERSVGPLTRKGFYL
AFQDIGACVALLSVRVYYKKCPELLQGLAHFPETIAGSDAPSLAT
VAGTCVDHAVVPPGGEEPRMHCAVDGEWLVPIGQCLCQAGYEKVE
DACQACSPGFFKFEASESPCLECPEHTLPSPEGATSCECEEGFFR
APQDPASMPCTRPPSAPHYLTAVGMGAKVELRWTPPQDSGGREDI
VYSVTCEQCWPESGECGPCEASVRYSEPPHGLTRTSVTVSDLEPH
MNYTFTVEARNGVSGLVTSRSFRTASVSINQTEPPKVRLEGRSTT
SLSVSWSIPPPQQSRVWKYEVTYRKKGDSNSYNVRRTEGFSVTLD
DLAPDTTYLVQVQALTQEGQGAGSKVHEFQTLSPEGSGNLAVIGG
VAVGVVLLLVLAGVGFFIHRRRKNQRARQSPEDVYFSKSEQLKPL
KTYVDPHTYEDPNQAVLKFTTEIHPSCVTRQKVIGAGEFGEVYKG
MLKTSSGKKEVPVAIKTLKAGYTEKQRVDFLGEAGIMGQFSHHNI
IRLEGVISKYKPMMIITEYMENGALDKFLREKDGEFSVLQLVGML
RGIAAGMKYLANMNYVHRDLAARNILVNSNLVCKVSDFGLSRVLE
DDPEATYTTSGGKIPIRWTAPEAISYRKFTSASDVWSFGIVMWEV
MTYGERPYWELSNHEVMKAINDGFRLPTPMDCPSAIYQLMMQCWQ
QERARRPKFADIVSILDKLIRAPDSLKTLADFDPRVSIRLPSTSG
SEGVPFRTVSEWLESIKMQQYTEHFMAAGYTAIEKVVQMTNDDIK
RIGVRLPGHQKRIAYSLLGLKDQVNTVGIPI

| | | |
|---|---|---|
| PRKAR1A | P10644 | CAMP-dependent protein |
| | | kinase type I-alpha |
| | | regulatory subunit |

Nucleotide Sequence (Seq ID No. 20):
>P000113_CAN_CAN1-1_PRKAR1A_5573_Homo sapiens
protein kinase cAMP-dependent regulatory
type I alpha (tissue specific e_BC036285.1_
AAH36285.1_P10644_0_0_1146_0_1143
ATGGAGTCTGGCAGTACCGCCGCCAGTGAGGAGGCACGCAGCCTT
CGAGAATGTGAGCTCTACGTCCAGAAGCATAACATTCAAGCGCTG
CTCAAAGATTCTATTGTGCAGTTGTGCACTGCTCGACCTGAGAGA
CCCATGGCATTCCTCAGGGAATACTTTGAGAGGTTGGAGAAGGAG
GAGGCAAAACAGATTCAGAATCTGCAGAAAGCAGGCACTCGTACA
GACTCAAGGGAGGATGAGATTTCTCCTCCTCCACCCAACCCAGTG
GTTAAAGGTAGGAGGCGACGAGGTGCTATCAGCGCTGAGGTCTAC
ACGGAGGAAGATGCGGCATCCTATGTTAGAAAGGTTATACCAAAA
GATTACAAGACAATGGCCGCTTTAGCCAAAGCCATTGAAAAGAAT
GTGCTGTTTTCACATCTTGATGATAATGAGAGAAGTGATATTTTT
GATGCCATGTTTTCGGTCTCCTTTATCGCAGGAGAGACTGTGATT
CAGCAAGGTGATGAAGGGGATAACTTCTATGTGATTGATCAAGGA
GAGACGGATGTCTATGTTAACAATGAATGGGCAACCAGTGTTGGG
GAAGGAGGGAGCTTTGGAGAACTTGCTTTGATTTATGGAACACCG
AGAGCAGCCACTGTCAAAGCAAAGACAAATGTGAAATTGTGGGGC
ATCGACCGAGACAGCTATAGAAGAATCCTCATGGGAAGCACACTG
AGAAAGCGGAAGATGTATGAGGAATTCCTTAGTAAAGTCTCTATT
TTAGAGTCTCTGGACAAGTGGGAACGTCTTACGGTAGCTGATGCA
TTGGAACCAGTGCAGTTTGAAGATGGGCAGAAGATTGTGGTGCAG
GGAGAACCAGGGGATGAGTTCTTCATTATTTTAGAGGGGTCAGCT
GCTGTGCTACAACGTCGGTCAGAAAATGAAGAGTTTGTTGAAGTG
GGAAGATTGGGGCCTTCTGATTATTTTGGTGAAATTGCACTACTG
ATGAATCGTCCTCGTGCTGCCACAGTTGTTGCTCGTGGCCCCTTG
AAGTGCGTTAAGCTGGACCGACCTAGATTTGAACGTGTTCTTGGC
CCATGCTCAGACATCCTCAAACGAAACATCCAGCAGTACAACAGT
TTTGTGTCACTGTCTGTC
Protein Sequence (Seq ID No. 41):
>sp|P10644|KAPO_HUMAN CAMP-dependent protein
kinase type I-alpha regulatory subunit
OS = Homo sapiens
OX = 9606 GN = PRKAR1A PE = 1 SV = 1
MESGSTAASEEARSLRECELYVQKHNIQALLKDSIVQLCTARPER
PMAFLREYFERLEKEEAKQIQNLQKAGTRTDSREDEISPPPPNPV
VKGRRRRGAISAEVYTEEDAASYVRKVIPKDYKTMAALAKAIEKN
VLFSHLDDNERSDIFDAMFSVSFIAGETVIQQGDEGDNFYVIDQG
ETDVYVNNEWATSVGEGGSFGELALIYGTPRAATVKAKTNVKLWG
IDRDSYRRILMGSTLRKRKMYEEFLSKVSILESLDKWERLTVADA
LEPVQFEDGQKIVVQGEPGDEFFIILEGSAAVLQRRSENEEFVEV
GRLGPSDYFGEIALLMNRPRAATVVARGPLKCVKLDRPRFERVLG
PCSDILKRNIQQYNSFVSLSV

| | | |
|---|---|---|
| EAPP | Q56P03 | E2F-associated |
| | | phosphoprotein |

Nucleotide Sequence (Seq ID No. 21):
>P001616_Q106_Q106p2_EAPP_55837_Homo sapiens
chromosome 14 open reading frame
11_BC001245.1_AAH01245.1_xx_0_0_858_0_855
ATGAACCGGCTTCCGGATGACTACGACCCCTACGCGGTTGAAGAG
CCTAGCGACGAGGAGCCGGCTTTGAGCAGCTCTGAGGATGAAGTG
GATGTGCTTTTACATGGAACTCCTGACCAAAAACGAAAACTCATC
AGAGAATGTCTTACCGGAGAAAGTGAATCATCTAGTGAAGATGAA
TTTGAAAAGGAGATGGAAGCTGAATTAAATTCTACCATGAAAACA
ATGGAGGACAAGTTATCCTCTCTGGGAACTGGATCTTCCTCAGGA
AATGGAAAAGTTGCAACAGCTCCGACAAGGTACTACGATGATATA
TATTTTGATTCTGATTCCGAGGATGAAGACAGAGCAGTACAGGTG
ACCAAGAAAAAAAAGAAGAAACAACACAAGATTCCAACAAATGAC
GAATTACTGTATGATCCTGAAAAAGATAACAGAGATCAGGCCTGG
GTTGATGCACAGAGAAGGGGTTACCATGGTTTGGGACCACAGAGA
TCACGTGAACAACAGCCTGTTCCAAATAGTGATGCTGTCTTGAAT
TGTCCTGCCTGCATGACCACACTTTGCCTTGATTGCCAAAGGCAT
GAATCATACAAAACTCAATATAGAGCAATGTTTGTAATGAATTGT
TCTATTAACAAAGAGGAGGTTCTAAGATATAAAGCCTCAGAGAAC
AGGAAGAAAAGGCGGGTCCATAAGAAGATGAGGTCTAACCAGGAA
GATGCTGCTGAGAAGGCAGAGACAGATGTGGAAGAAATCTATCAC
CCAGTCATGTGCACTGAATGTTCCACTGAAGTGGCAGTCTACGAC
AAGGATGAAGTCTTTCATTTTTTCAATGTTTTAGCAAGCCATTCC
Protein Sequence (Seq ID No. 42):
>sp|Q56P03|EAPP_HUMAN E2F-associated
phosphoprotein
OS = Homo sapiens OX = 9606 GN = EAPP PE = 1
SV = 4
MNRLPDDYDPYAVEEPSDEEPALSSSEDEVDVLLHGTPDQKRKLI
RECLTGESESSSEDEFEKEMEAELNSTMKTMEDKLSSLGTGSSSG
NGKVATAPTRYYDDIYFDSDSEDEDRAVQVTKKKKKKQHKIPTND
ELLYDPEKDNRDQAWVDAQRRGYHGLGPQRSRQQQPVPNSDAVLN
CPACMTTLCLDCQRHESYKTQYRAMFVMNCSINKEEVLRYKASEN
RKKRRVHKKMRSNREDAAEKAETDVEEIYHPVMCTECSTEVAVYD
KDEVFHFFNVLASHS

1. A method for predicting a response to adalimumab from a serum/plasma sample extracted from a rheumatoid arthritis patient prior to treating the patient with adalimumab, said response being classified as a good response corresponding to anti-drug antibody negative or a poor response corresponding to anti-drug antibody positive, comprising the steps of:
(i) testing the sample for the presence of autoantibody biomarkers; and
(ii) determining whether the patient will develop a good response or a poor response to treatment with adalimumab, based on the detection of said autoantibody biomarkers:
characterised in that said autoantibody biomarkers comprise autoantibodies to antigens SSB, TROVE2 and ZHX2, wherein ZHX2 is associated with the good response, and SSB and TROVE2 are associated with the poor response.

2. The method according to claim 1 wherein the autoantibody biomarkers further comprise autoantibodies to one or more antigens from the group comprising of: PPARD, TRIB2, SH3GL1, ODC1, EEF1D, PRKARIA and EAPP which are associated with the good response, and SPANXN2, HNRNPA2B, CEP55, FN3K, PANK3. HPCAL1. THRA. AIFM1, RPS6KA4, KLF10 and EPHA2 which are associated with the poor response.

3. The method according to claim 1 wherein the antigens are biotinylated proteins.

4. The method according to claim 3 wherein each biotinylated protein is formed from a Biotin Carboxyl Carrier Protein folding marker which is fused in-frame with a protein.

5. The method according to claim 3 wherein the biotinylated proteins are bound to a streptavidin-coated substrate.

6. The method according to claim 5 wherein the substrate comprises a hydrogel-forming polymer base layer.

7. The method according to claim 1 wherein the antigens are exposed to a serum/plasma sample extracted from a rheumatoid arthritis patient, such that autoantibody biomarkers from the sample may bind to the antigens.

8. The method according to claim 7 wherein the antigens are subsequently exposed to a fluorescently-tagged secondary antibody to allow the amount of any autoantibodies from the sample bound to the antigens to be determined.

9. The method according to claim 8 wherein the patient's response to treatment with adalimumab corresponds to the relative or absolute amount of autoantibodies from the sample specifically binding to the antigens.

10. The method according to claim 1 wherein the steps are performed in vitro.

11. The method according to claim 1 wherein the method comprises detecting upregulation/downregulation of one or more biomarkers

12. Use of a kit to predict a response to adalimumab from a serum/plasma sample extracted from a rheumatoid arthritis patient prior to treating the patient with adalimumab, said response being classified as a good response corresponding to anti-drug antibody negative or a poor response corresponding to anti-drug antibody positive, the method of manufacturing the kit comprising the steps of:
for each antigen in a panel, cloning a biotin carboxyl carrier protein folding marker in-frame with a gene encoding the antigen and expressing the resulting biotinylated antigen;
binding the biotinylated antigens to addressable locations on one or more streptavidin-coated substrates, thereby forming an antigen array;
such that the amount of autoantibodies from the sample binding to the antigens on the panel can be determined by exposing the substrate to the sample and measuring the response;
characterised in that the antigens comprise SSB, TROVE2 and ZHX2, wherein ZHX2 is associated with the good response, and SSB and TROVE2 are associated with the poor response.

13. The use according to claim 12 wherein the antigens further comprise one or more of: PPARD, TRIB2, SH3GL1, ODC1. EEF1D, PRKAR1A and EAPP which are associated with the good response, and SPANXN2. HNRNPA2B, CEP55, FN3K, PANK3, HPCAL1. THRA, AIFMI1, RPS6KA4, KLF10 and EPHA2 which are associated with the poor response.

14. Use of a composition comprising a panel of antigens to predict an immunogenic and/or therapeutic response to adalimumab in a rheumatoid arthritis patient who has not previously been treated with adalimumab, said response being classified as a good response corresponding to anti-drug antibody negative or a poor response corresponding to anti-drug antibody positive, characterised in that the antigens comprise SSB, TROVE2 and ZHX2, wherein ZHX2 is associated with the good response, and SSB and TROVE2 are associated with the poor response.

15. The use according to claim 14 wherein the antigens further comprise one or more of PPARD. TRIB2, SH3GL1, ODC1. EEF1D. PRKAR1A and EAPP which are associated with the good response, and SPANXN2, HNRNPA2B. CEP55. FN3K, PANK3, HPCAL1, THRA, AIFM1, RPS6KA4. KLF10 and EPHA2 and which are associated with the poor response.

16. The use according to claim 14 wherein the antigens are biotinylated proteins.

17. The use according to claim 14 wherein the amount of one or more autoantibody biomarkers binding in vitro to the antigens in a sample from a patient can be measured to predict the response.

18. Use of a composition comprising a panel of autoantibody biomarkers to predict an immunogenic and/or therapeutic response to adalimumab in a rheumatoid arthritis patient who has not previously been treated with adalimumab, said response being classified as a good response corresponding to anti-drug antibody negative or a poor response corresponding to anti-drug antibody positive,
wherein the level of the autoantibody biomarkers are measured in a serum/plasma sample collected from the patient:
characterised in that the autoantibody biomarkers are specific to antigens comprising SSB, TROVE2 and ZHX2, wherein ZHX2 is associated with the good response, and SSB and TROVE2 are associated with the poor response.


SEQUENCE LISTING (42 sequences)

SEQ ID NO: 1 - DNA, 1224 bp, Escherichia coli
atggctgagaacggcgacaacgagaagatggctgctctcgaggctaagatctgccaccagatcgagtactacttcggcgacttcaacctgccccgtgacaagttcctgaaggaacagatcaagctggacgagggctgggtgcccctcgagatcatgatcaagttcaaccgtctgaaccgcctgaccaccgacttcaacgtgatcgtcgaggctctgtccaagtccaaggctgagctgatggaaatctccgaggacaagaccaagatccgtcgttccccatccaagcccctgcccgaagtgaccgacgagtacaagaacgacgtgaagaaccgttccgtgtacatcaagggtttccccaccgacgctaccctggacgacatcaaggaatggctcgaggacaagggccaggtgctgaacatccagatgcgtcgtaccctgcacaaggctttcaagggttccatcttcgtggtgttcgactccatcgagtccgctaagaagttcgtcgagactcccggccagaagtacaaggaaaccgacctgctgatcctgttcaaggacgactacttcgccaagaagaacgaggaacgcaagcagaacaaggtcgaggccaagctgcgtgctaagcaagagcaagaggctaagcagaagctggaagaggacgctgagatgaagtccctggaagagaagatcggttgcctgctgaagttctccggcgacctggacgaccagacctgccgcgaggacctgcacatcctgttctccaaccacggcgagatcaagtggatcgacttcgtgcgtggtgctaaggaaggcatcatcctcttcaaggaaaaggccaaggaagctctgggcaaggctaaggacgctaacaacggcaacctgcagctgcgtaacaaggaagtgacctgggaggtcctcgagggcgaggtcgagaaggaagccctgaagaagatcatcgaggaccagcaagagtccctgaacaagtggaagtccaagggtcgtcgtttcaagggcaagggaaagggcaacaaggctgctcagcccggttccggaaagggaaaggtgcagttccagggcaagaagaccaagttcgcttccgacgacgagcacgatgagcacgacgagaacggtgctaccggtcccgtgaagcgtgctcgtgaagagactgacaaggaagaacccgcttccaagcagcaaaagaccgagaacggcgctggcgaccag

SEQ ID NO: 2 - DNA, 1614 bp, Escherichia coli
atggaggaatctgtaaaccaaatgcagccactgaatgagaagcagatagccaattctcaggatggatatgtatggcaagtcactgacatgaatcgactacaccggttcttatgtttcggttctgaaggtgggacttattatatcaaagaacagaagttgggccttgaaaatgctgaagctttaattagattgattgaagatggcagaggatgtgaagtgatacaagaaataaagtcatttagtcaagaaggcagaaccacaaagcaagagcctatgctctttgcacttgccatttgttcccagtgctccgacataagcacaaaacaagcagcatttaaagctgtttctgaagtttgtcgcattcctacccatctctttacttttatccagtttaagaaagatctgaaggaaagcatgaaatgtggcatgtggggtcgtgccctccggaaggctatagcggactggtacaatgagaaaggtggcatggcccttgctctggcagttacaaaatataaacagagaaatggctggtctcacaaagatctattaagattgtcacatcttaaaccttccagtgaaggacttgcaattgtgaccaaatatattacaaagggctggaaagaagttcatgaattgtataaagaaaaagcactctctgtggagactgaaaaattattaaagtatctggaggctgtagagaaagtgaagcgcacaagagatgagctagaagtcattcatctaatagaagaacatagattagttagagaacatcttttaacaaatcacttaaagtctaaagaggtatggaaggctttgttacaagaaatgccgcttactgcattactaaggaatctaggaaagatgactgctaattcagtacttgaaccaggaaattcagaagtatctttagtatgtgaaaaactgtgtaatgaaaaactattaaaaaaggctcgtatacatccatttcatattttgatcgcattagaaacttacaagacaggtcatggtctcagagggaaactgaagtggcgccctgatgaagaaattttgaaagcattggatgctgctttttataaaacatttaagacagttgaaccaactggaaaacgtttcttactagctgttgatgtcagtgcttctatgaaccaaagagttttgggtagtatactcaacgctagtacagttgctgcagcaatgtgcatggttgtcacacgaacagaaaaagattcttatgtagttgctttttccgatgaaatggtaccatgtccagtgactacagatatgaccttacaacaggttttaatggctatgagtcagatcccagcaggtggaactgattgctctcttccaatgatctgggctcagaagacaaacacacctgctgatgtcttcattgtattcactgataatgagacctttgctggaggtgtccatcctgctattgctctgagggagtatcgaaagaaaatggatattccagctaaattgattgtttgtggaatgacatcaaatggtttcaccattgcagacccagatgatagaggcatgttggatatgtgcggctttgatactggagctctggatgtaattcgaaatttcacattagatatgatt

SEQ ID NO: 3 - DNA, 2511 bp, Escherichia coli
atggctagcaaacgaaaatctacaactccatgcatggttcggacatcacaagtagtagaacaagatgtgcccgaggaagtagacagggccaaagagaaaggaatcggcacaccacagcctgacgtggccaaggacagttgggcagcagaacttgaaaactcttccaaagaaaacgaagtgatagaggtgaaatctatgggggaaagccagtccaaaaaactccaaggtggttatgagtgcaaatactgcccctactccacgcaaaacctgaacgagttcacggagcatgtcgacatgcagcatcccaacgtgattctcaaccccctctacgtgtgtgcagaatgtaacttcacaaccaaaaagtacgactccctatccgaccacaactccaagttccatcccggggaggccaacttcaagctgaagttaattaaacgcaataatcaaactgtcttggaacagtccatcgaaaccaccaaccatgtcgtgtccatcaccaccagtggccctggaactggtgacagtgattctgggatctcggtgagtaaaacccccatcatgaagcctggaaaaccaaaagcggatgccaagaaggtgcccaagaagcccgaggagatcacccccgagaaccacgtggaagggaccgcccgcctggtgacagacacagctgagatcctctcgagactcggcggggtggagctcctccaagacacattaggacacgtcatgccttctgtacagctgccaccaaatatcaaccttgtgcccaaggtccctgtcccactaaatactaccaaatacaactctgccctggatacaaatgccacgatgatcaactctttcaacaagtttccttacccgacccaggctgagttgtcctggctgacagctgcctccaaacacccagaggagcacatcagaatctggtttgccacccagcgcttaaagcatggcatcagctggtccccagaagaggtggaggaggcccggaagaagatgttcaacggcaccatccagtcagtacccccgaccatcactgtgctgcccgcccagttggcccccacaaaggtgacgcagcccatcctccagacggctctaccgtgccagatcctcggccagactagcctggtgctgactcaggtgaccagcgggtcaacaaccgtctcttgctcccccatcacacttgccgtggcaggagtcaccaaccatggccagaagagacccttggtgactccccaagctgcccccgaacccaagcgtccacacatcgctcaggtgccagagcccccacccaaggtggccaaccccccgctcacaccagccagtgaccgcaagaagacaaaggagcagatagcacatctcaaggccagctttctccagagccagttccctgacgatgccgaggtttaccggctcatcgaggtgactggccttgccaggagcgagatcaagaagtggttcagtgaccaccgatatcggtgtcaaaggggcatcgtccacatcaccagcgaatcccttgccaaagaccagttggccatcgcggcctcccgacacggtcgcacgtatcatgcgtacccagactttgccccccagaagttcaaagagaaaacacagggtcaggttaaaatcttggaagacagctttttgaaaagttcttttcctacccaagcagaactggatcggctaagggtggagaccaagctgagcaggagagagatcgactcctggttctcggagaggcggaagcttcgagacagcatggaacaagctgtcttggattccatggggtctggcaaaaaaggccaagatgtgggagcccccaatggtgctctgtctcgactcgaccagctctccggtgcccagttaacaagttctctgcccagcccttcgccagcaattgcaaaaagtcaagaacaggttcatctcctgaggagcacgtttgcaagaacccagtggcctactccccaggagtacgaccagttagcggccaagactggcctggtccgaactgagattgtgcgttggttcaaggagaacagatgcttgctgaaaacgggaaccgtgaagtggatggagcagtaccagcaccagcccatggcagatgatcacggctacgatgccgtagcaaggaaagcaacaaaacccatggccgagagcccaaagaacgggggtgatgtggttccacaatattacaaggaccccaaaaagctctgcgaagaggacttggagaagttggtgaccagggtaaaagtaggcagcgagccagcaaaagactgtttgccagcaaagccctcagaggccacctcagaccggtcagagggcagcagccgggacggccagggtagcgacgagaacgaggagtcgagcgttgtggattacgtggaggtgacggtcggggaggaggatgcgatctcagatagatcagatagctggagtcaggctgcggcagaaggtgtgtcggaactggctgaatcagactccgactgcgtccctgcagaggctggccaggcc

SEQ ID NO: 4 - DNA, 1083 bp, Escherichia coli
atggagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaatgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggaggtgag

SEQ ID NO: 5 - DNA, 539 bp, Escherichia coli
tggaacagcccacctcttccaccaacggcgagaagcgcaagtccccctgcgagtccaacaacaagaaaaacgacgagatgcaagaggctcccaaccgtgtgctggctcccaagcagtccctgcaaaagaccaagaccatcgagtacctgaccatcatcgtgtactactaccgcaagcacaccaagatcaactccaaccagctcgagaaggaccagtcccgcgagaactccatcaaccccgtgcaagaggaagaggacgagggcctggactccgctgagggatcctcccaagaagatgaggacctggacagctccgagggttccagccaagaggatgaagatctcgactcctccgagggcagctcccaagaggacgaggacttggattcctccgagggatctagtcaagaggacgaggatctggactcttccgaaggctcatctcaagaagatgaagatttggacccccctgagggtagcagtcaagaggatgaggacctcgattccagcgagggctcctcacaagagggtggcgaggat

SEQ ID NO: 6 - DNA, 1023 bp, Escherichia coli
atggaacgcgagaaagagcagttccgcaagctgttcatcggtggcctgtccttcgagactaccgaggaatccctgcgcaactactacgagcagtggggcaagctgaccgactgcgtggtcatgcgtgaccccgcttccaagcgttcccgtggtttcggtttcgtgaccttctccagcatggctgaggtggacgctgctatggctgctcgtccccactccatcgacggtcgtgtggtcgagcctaagcgtgctgtggctcgtgaagagtccggcaagcctggtgctcacgtgaccgtgaagaagctgttcgttggcggtatcaaagaggacaccgaggaacaccacctgagggactacttcgaggaatacggcaagatcgacaccatcgagatcatcaccgaccgtcagtccggaaagaagcgcggcttcggcttcgtcactttcgacgaccacgaccccgtggacaagatcgtgctgcagaagtaccacaccatcaacggtcacaacgctgaagtgcgcaaggctctgtcccgtcaagagatgcaagaggtgcagtcctcccgttccggtcgtggtggcaacttcggattcggcgactctcgcggtggtggcggaaacttcggtcctggtcccggttccaacttccgtggtggttccgacggttacggctccggaagaggtttcggcgacggctacaacggctacggtggtggtcctggcggtggaaatttcggtggttcccctggttacggtggcggtcgcggtggatacggcggaggtggtccaggatacggcaaccagggtggcggttacggcggtggttacgacaactacggtggcggcaactacggttccggaaactacaacgacttcggcaattacaaccagcagccctccaactacggccccatgaagtctggcaatttcggcggctcccgtaacatgggtggtccttacggtggtggaaattacggtcccggtggttccggtggctctggtggctacggcggtcgttcccgttac

SEQ ID NO: 7 - DNA, 1029 bp, Escherichia coli
atgaacatacacaggtctacccccatcacaatagcgagatatgggagatcgcggaacaaaacccaggatttcgaagagttgtcgtctataaggtccgcggagcccagccagagtttcagcccgaacctcggctccccgagcccgcccgagactccgaacttgtcgcattgcgtttcttgtatcgggaaatacttattgttggaacctctggagggagaccacgtttttcgtgccgtgcatctgcacagcggagaggagctggtgtgcaaggtgtttgatatcagctgctaccaggaatccctggcaccgtgcttttgcctgtctgctcatagtaacatcaaccaaatcactgaaattatcctgggtgagaccaaagcctatgtgttctttgagcgaagctatggggacatgcattccttcgtccgcacctgcaagaagctgagagaggaggaggcagccagactgttctaccagattgcctcggcagtggcccactgccatgacggggggctggtgctgcgggacctcaagctgcggaaattcatctttaaggacgaagagaggactcgggtcaagctggaaagcctggaagacgcctacattctgcggggagatgatgattccctctccgacaagcatggctgcccggcttacgtaagcccagagatcttgaacaccagtggcagctactcgggcaaagcagccgacgtgtggagcctgggggtgatgctgtacaccatgttggtggggcggtaccctttccatgacattgaacccagctccctcttcagcaagatccggcgtggccagttcaacattccagagactctgtcgcccaaggccaagtgcctcatccgaagcattctgcgtcgggagccctcagagcggctgacctcgcaggaaattctggaccatccttggttttctacagattttagcgtctcgaattcagcatatggtgctaaggaagtgtctgaccagctggtgccggacgtcaacatggaagagaacttggaccctttctttaac

SEQ ID NO: 8 - DNA, 1392 bp, Escherichia coli
atgtcctcccgttccaccaaggacctgatcaagtctaagtggggttccaagccctccaactccaagtccgagactaccctcgagaagctgaagggcgagatcgctcacctcaagacctccgtggacgagatcacctccggcaagggcaagctgaccgacaaggaacgtcaccgtctgctcgagaagatccgtgtgctcgaggctgagaaggaaaagaacgcttaccagctgactgagaaggacaaggaaatccagcgtctgcgcgaccagctgaaggctcgttactccaccaccgctctgctggaacagctggaagaaaccacccgcgagggcgagcgtcgcgagcaggtcctgaaggctctgtccgaagagaaggacgtgctgaagcagcagctgtccgctgctacctcccgtatcgctgagctggaatccaagaccaacaccctgcgtctgtcccagaccgtggctcccaactgcttcaactcctccatcaacaacatccacgagatggaaatccaactgaaggacgctctcgagaagaaccagcagtggctggtgtacgaccagcagcgcgaggtgtacgtgaagggcctgctggctaagatcttcgagctggaaaagaagaccgagactgctgctcactccctgccccagcagaccaagaagcccgagtccgagggttacctgcaagaggaaaagcagaagtgctacaacgacctgctggcttccgctaagaaggacctggaagtcgagcgtcagaccatcacccagctgtccttcgagctgtccgagttccgtaggaagtacgaagagactcagaaggaagtccacaacctgaaccagctgctgtactcccagcgtcgtgctgacgtgcagcacctcgaggacgaccgtcacaagactgagaagatccagaagctgcgcgaagagaacgatatcgctcgtggcaagctcgaggaagagaagaagcgttccgaggaactgctgtcccaggtgcagttcctgtacacctccctgctcaagcagcaagaggaacagacccgtgtggctctgttggagcagcagatgcaggcttgcaccctggacttcgagaacgagaagctggaccgtcagcacgtccagcaccagctgcacgtgatcctgaaggaactgcgcaaggctcgtaaccagatcacccagttggagtccctgaagcagctgcacgagttcgctatcaccgagcccctggtcaccttccaaggcgagactgagaaccgcgagaaggtggccgcttcccccaagtcccccaccgctgctctgaacgagtccctggtcgagtgccccaagtgcaacatccagtaccccgctaccgagcaccgtgacctgctggtgcacgtcgagtactgctccaag

SEQ ID NO: 9 - DNA, 1104 bp, Escherichia coli
atgtcggtggcggggctgaagaagcagttctacaaggcgagccagctggtcagtgagaaggtcggaggggccgaggggaccaagctggatgatgacttcaaagagatggagaagaaggtggatgtcaccagcaaggcggtgacagaagtgctggccaggaccatcgagtacctgcagcccaacccagcctcgcgggctaagctgaccatgctcaacacggtgtccaagatccggggccaggtgaagaaccccggctacccgcagtcggaggggcttctgggcgagtgcatgatccgccacgggaaggagctgggcggcgagtccaactttggtgacgcattgctggatgccggcgagtccatgaagcgcctggcagaggtgaaggactccctggacatcgaggtcaagcagaacttcattgaccccctccagaacctgtgcgagaaagacctgaaggagatccagcaccacctgaagaaactggagggccgccgcctggactttgactacaagaagaagcggcagggcaagatccccgatgaggagctacgccaggcgctggagaagttcgaggagtccaaggaggtggcagaaaccagcatgcacaacctcctggagactgacatcgagcaggtgagtcagctctcggccctggtggatgcacagctggactaccaccggcaggccgtgcagatcctggacgagctggcggagaagctcaagcgcaggatgcgggaagcttcctcacgccctaagcgggagtataagccgaagccccgggagccctttgaccttggagagcctgagcagtccaacgggggcttcccctgcaccacagcccccaagatcgcagcttcatcgtctttccgatcttccgacaagcccatccggacccctagccggagcatgccgcccctggaccagccgagctgcaaggcgctgtacgacttcgagcccgagaacgacggggagctgggcttccatgagggcgacgtcatcacgctgaccaaccagatcgatgagaactggtacgagggcatgctggacggccagtcgggcttcttcccgctcagctacgtggaggtgcttgtgcccctgccgcag

SEQ ID NO: 10 - DNA, 927 bp, Escherichia coli
atggagcagctgctgcgcgccgagctgcgcaccgcgaccctgcgggccttcggcggccccggcgccggctgcatcagcgagggccgagcctacgacacggacgcaggcccagtgttcgtcaaagtcaaccgcaggacgcaggcccggcagatgtttgagggggaggtggccagcctggaggccctccggagcacgggcctggtgcgggtgccgaggcccatgaaggtcatcgacctgccgggaggtggggccgcctttgtgatggagcatttgaagatgaagagcttgagcagtcaagcatcaaaacttggagagcagatggcagatttgcatctttacaaccagaagctcagggagaagttgaaggaggaggagaacacagtgggccgaagaggtgagggtgctgagcctcagtatgtggacaagttcggcttccacacggtgacgtgctgcggcttcatcccgcaggtgaatgagtggcaggatgactggccgacctttttcgcccggcaccggctccaggcgcagctggacctcattgagaaggactatgctgaccgagaggcacgagaactctggtcccggctacaggtgaagatcccggatctgttttgtggcctagagattgtccccgcgttgctccacggggatctctggtcgggaaacgtggctgaggacgacgtggggcccattatttacgacccggcttccttctatggccattccgagtttgaactggcaatcgccttgatgtttggggggttccccagatccttcttcaccgcctaccaccggaagatccccaaggctccgggcttcgaccagcggctgctgctctaccagctgtttaactacctgaaccactggaaccacttcgggcgggagtacaggagcccttcgttgggcaccatgcgaaggctgctcaag

SEQ ID NO: 11 - DNA, 1110 bp, Escherichia coli
atgaagatcaaagatgccaagaaaccctctttcccatggtttggcatggacattgggggaactctagtaaagctctcgtactttgaacctattgatatcacagcagaggaagagcaagaagaagttgagagtttaaaaagtattcggaaatatttgacttctaacgtggcatatggatccaccggcattcgggatgtacaccttgaactgaaagatttaacactttttggccgaagagggaacttgcactttatcaggtttccaacccaggacctgcctacttttatccaaatgggaagagataaaaacttctcaacattgcagacggtgctatgtgctacaggaggtggtgcttacaagtttgaaaaagattttcgcacaattggaaacctccacctgcacaaactggatgaacttgactgccttgtaaagggcttgctgtatatagactctgtcagtttcaatggacaagccgagtgctattattttgctaatgcctcagaacctgagcgatgccaaaagatgccttttaacctggatgatccctatccactgcttgtagtgaacattggctcaggagtcagtattttagcagtccattccaaagacaactataaacgagtgactgggacaagccttggagggggtacctttctgggtttatgcagtttattgactggctgtgaaagttttgaagaggctcttgaaatggcatccaaaggtgatagcacacaagctgacaagctggtccgtgatatttatggaggagattatgaaagatttggtttgccaggttgggctgtagcatctagttttgggaatatgatttataaggagaagcgagaatctgttagtaaagaagatctggcaagagctactttagttactatcaccaataacattggttctgtggcacgaatgtgtgctgttaatgagaaaataaacagagttgtctttgttggaaactttttacgtgtcaataccctctcaatgaaacttttggcatatgcactggattactggtcaaaaggtcaactaaaagcattgtttctagaacatgagggttactttggagcagttggtgcacttcttgggctgccaaatttcagc

SEQ ID NO: 12 - DNA, 579 bp, Escherichia coli
atgggcaagcagaactccaagctgcgtcccgaggtgctgcaggacctgcgcgagaacaccgagttcaccgaccacgagctgcaagagtggtacaagggtttcctgaaggactgccccaccggtcacctgaccgtggacgagttcaagaagatctacgctaacttcttcccctacggcgacgcttccaagttcgctgagcacgtgttccgtaccttcgacaccaacggcgacggcaccatcgacttccgcgagttcatcatcgctctgtccgtgacctcccgtggcaagctcgagcaaaagctgaagtgggctttctcgatgtacgacctggacggcaacggttacatctcccgttccgagatgctcgagatcgtgcaggctatctacaagatggtgtcctccgtgatgaagatgcccgaggacgagtccacccccgagaagcgtaccgacaagatcttccgtcagatggacaccaacaacgacggaaagctgtccctggaagagttcatccgtggtgctaagtccgacccctccatcgtgcgtctgctgcagtgcgacccatcctccgcttcccagttc

SEQ ID NO: 13 - DNA, 1470 bp, Escherichia coli
atggaacagaagccaagcaaggtggagtgtgggtcagacccagaggagaacagtgccaggtcaccagatggaaagcgaaaaagaaagaacggccaatgttccctgaaaaccagcatgtcagggtatatccctagttacctggacaaagacgagcagtgtgtcgtgtgtggggacaaggcaactggttatcactaccgctgtatcacttgtgagggctgcaagggcttctttcgccgcacaatccagaagaacctccatcccacctattcctgcaaatatgacagctgctgtgtcattgacaagatcacccgcaatcagtgccagctgtgccgcttcaagaagtgcatcgccgtgggcatggccatggacttggttctagatgactcgaagcgggtggccaagcgtaagctgattgagcagaaccgggagcggcggcggaaggaggagatgatccgatcactgcagcagcgaccagagcccactcctgaagagtgggatctgatccacattgccacagaggcccatcgcagcaccaatgcccagggcagccattggaaacagaggcggaaattcctgcccgatgacattggccagtcacccattgtctccatgccggacggagacaaggtggacctggaagccttcagcgagtttaccaagatcatcaccccggccatcacccgtgtggtggactttgccaaaaaactgcccatgttctccgagctgccttgcgaagaccagatcatcctcctgaaggggtgctgcatggagatcatgtccctgcgggcggctgtccgctacgaccctgagagcgacaccctgacgctgagtggggagatggctgtcaagcgggagcagctcaagaatggcggcctgggcgtagtctccgacgccatctttgaactgggcaagtcactctctgcctttaacctggatgacacggaagtggctctgctgcaggctgtgctgctaatgtcaacagaccgctcgggcctgctgtgtgtggacaagatcgagaagagtcaggaggcgtacctgctggcgttcgagcactacgtcaaccaccgcaaacacaacattccgcacttctggcccaagctgctgatgaaggagagagaagtgcagagttcgattctgtacaagggggcagcggcagaaggccggccgggcgggtcactgggcgtccacccggaaggacagcagcttctcggaatgcatgttgttcagggtccgcaggtccggcagcttgagcagcagcttggtgaagcgggaagtctccaagggccggttcttcagcaccagagcccgaagagcccgcagcagcgtctcctggagctgctccaccgaagcggaattctccatgcccgagcggtctgtggggaagacgacagcagtgaggcggactccccgagctcctctgaggaggaaccggaggtctgcgaggacctggcaggcaatgcagcctctccc

SEQ ID NO: 14 - DNA, 783 bp, Escherichia coli
atggaaaaggtccgccgcgagggtgtcaaggtcatgcccaacgctatcgtgcagtccgtgggcgtgtcctccggcaagctgctgatcaagctgaaggacggtcgcaaggtggaaaccgaccacatcgtggctgctgtgggcctcgagcccaacgtcgagctggctaagaccggtggcctcgagatcgactccgacttcggtggtttccgtgtgaacgctgagctgcaggctcgttccaacatctgggtggccggcgacgctgcttgcttctacgacatcaagctgggtcgtcgtcgtgtcgagcaccacgaccacgctgtggtgtccggtcgtctggctggcgagaacatgaccggtgctgctaagccctactggcaccagtccatgttctggtccgacctgggtcccgacgtgggttacgaggctatcggcctggtggactcctccctgcccaccgtgggagtgttcgctaaggctaccgctcaggacaaccccaagtccgctaccgagcagtccggcaccggtatccgttccgagtccgagactgagtccgaggcttccgagatcaccatccccccctccacccccgctgtgcctcaagctcctgtgcagggcgaggactacggcaagggtgtcatcttctacctgcgtgacaaggtggtcgtgggtatcgtgctgtggaacatcttcaaccgtatgcctatcgcccgcaagatcatcaaggacggcgagcagcacgaggacctgaacgaggtggccaagctgttcaacatccacgaggac

SEQ ID NO: 15 - DNA, 1383 bp, Escherichia coli
atgaacaactttggtaatgaagagtttgactgccacttcctcgatgaaggttttactgccaaggacattctggaccagaaaattaatgaagtttcttcttctgatgataaggatgccttctatgtggcagacctgggagacattctaaagaaacatctgaggtggttaaaagctctccctcgtgtcacccccttttatgcagtcaaatgtaatgatagcaaagccatcgtgaagacccttgctgctaccgggacaggatttgactgtgctagcaagactgaaatacagttggtgcagagtctgggggtgcctccagagaggattatctatgcaaatccttgtaaacaagtatctcaaattaagtatgctgctaataatggagtccagatgatgacttttgatagtgaagttgagttgatgaaagttgccagagcacatcccaaagcaaagttggttttgcggattgccactgatgattccaaagcagtctgtcgtctcagtgtgaaattcggtgccacgctcagaaccagcaggctccttttggaacgggcgaaagagctaaatatcgatgttgttggtgtcagcttccatgtaggaagcggctgtaccgatcctgagaccttcgtgcaggcaatctctgatgcccgctgtgtttttgacatgggggctgaggttggtttcagcatgtatctgcttgatattggcggtggctttcctggatctgaggatgtgaaacttaaatttgaagagatcaccggcgtaatcaacccagcgttggacaaatactttccgtcagactctggagtgagaatcatagctgagcccggcagatactatgttgcatcagctttcacgcttgcagttaatatcattgccaagaaaattgtattaaaggaacagacgggctctgatgacgaagatgagtcgagtgagcagacctttatgtattatgtgaatgatggcgtctatggatcatttaattgcatactctatgaccacgcacatgtaaagccccttctgcaaaagagacctaaaccagatgagaagtattattcatccagcatatggggaccaacatgtgatggcctcgatcggattgttgagcgctgtgacctgcctgaaatgcatgtgggtgattggatgctctttgaaaacatgggcgcttacactgttgctgctgcctctacgttcaatggcttccagaggccgacgatctactatgtgatgtcagggcctgcgtggcaactcatgcagcaattccagaaccccgacttcccacccgaagtagaggaacaggatgccagcaccctgcctgtgtcttgtgcctgggagagtgggatgaaacgccacagagcagcctgtgcttcggctagtattaatgtg

SEQ ID NO: 16 - DNA, 1572 bp, Escherichia coli
atgggggacgaggacgacgatgagagctgcgccgtggagctgcggatcacagaagccaacctgaccgggcacgaggagaaggtgagcgtggagaacttcgagctgctcaaggtgctgggcacgggagcctacggcaaggtgttcctggtgcggaaggcgggcgggcacgacgcggggaagctgtacgccatgaaggtgctgcgcaaggcggcgctggtgcagcgcgccaagacgcaggagcacacgcgcaccgagcgctcggtgctggagctggtgcgccaggcgcccttcctggtcacgctgcactacgctttccagacggatgccaagctgcacctcatcctggactatgtgagcggcggggagatgttcacccacctctaccagcgccagtacttcaaggaggctgaggtgcgcgtgtatgggggtgagatcgtgctggccctggaacacctgcacaagctcggcatcatttaccgagacctgaaactggagaatgtgctgctggactccgagggccacattgtcctcacggacttcgggctgagcaaggagttcctgacggaggagaaagagcggaccttctccttctgtggcaccatcgagtacatggcccccgaaatcatccgtagcaagacggggcatggcaaggctgtggactggtggagcctgggcatcttgctcttcgagctgctgacgggggcctcgcccttcaccctggagggcgagaggaacacgcaggctgaggtgtctcgacggatcctgaagtgctcccctcccttcccccctcggatcgggcccgtggcgcaggacctgctgcagcggctgctttgtaaggatcctaagaagcgattgggcgcggggccccagggggcacaagaagtccggaaccatcccttcttccagggcctcgattgggtggctctggctgccaggaagattccagccccattccggccccaaatccgctcagagctggatgtgggcaactttgcggaggaattcactcggctggagcctgtctactcaccccctggcagccccccacctggggacccccgaatctttcagggatactcctttgtggcaccctccattctctttgaccacaacaacgcggccgagatcatgtgcaaaatccgcgaggggcgcttctcccttgacggggaggcctggcagggtgtatccgaggaagccaaggagctggtccgagggctcctgaccgtggaccccgccaagcggctgaagctcgagggactgcggggcagctcgtggctgcaggacggcagcgcgcgctcctcgcccccgctccggacgcccgacgtgctcgagtcctctgggcccgcagtgcgctcgggtctcaacgccaccttcatggcattcaaccggggcaagcgggagggcttcttcctgaagagcgtggagaacgcacccctggccaagcggcggaagcagaagctgcggagcgccaccgcctcccgccggggctcccctgcaccagccaacccgggccgagcccccgtcgccgccaaaggggccccccgccgagccaacggccccctgcccccctcc

SEQ ID NO: 17 - DNA, 1941 bp, Escherichia coli
atgaggagcgggaaggcctcctgcaccctggagaccgtgtgggaagacaagcacaagtatgaggaggccgagcggcgcttctacgaacacgaggccacacaggcggccgcctccgcccagcagctgccagccgaggggccagccatgaatgggcccggccaggacgaccctgaggacgctgatgaggcggaagcccctgacggcggcagcaggcgtgatcccaggaagagccaggacagcaggaagcccctgcagaaaaagaggaagcgctcccccaagagcgggctcggccccgcggacctggccctcctgggcctctcggccgaacgtgtgtggctggacaagtcacttttcgaccaggcagagagctcctaccgccagaagctggcagatgtggctgcccaggcagcctggcctcctgccttggccccttggggtctctgcacccatggaaaccaggtggcctgccaccacgtgacctgggggatctgggtcaacaagtcctccttcgaccaggctgagcgggccttcgtggagtggtctcaggccctgttgctggcccccgagggcagccgcaggcaggggactcccaacacaggccagcaggtggccgtccccgacctggcccaccagcccagcccaccggtcaatggccagcccccgctgggcagcctgcaggcactggttcgggaggtgtggctggagaagccccggtatgatgcagccgagaggggcttctacgaggccctgtttgacggccatcccccagggaaggtgcgcctgcaagagcgagccggcctggccgagggtgcccggcggggccgcagagaccggcggggccgcaacatcttagggaacaagcgggccgggctgcgacgggccgatggggaggccccctctgccttgccctactgttacttcctgcagaaggatgcagaggccccctggctcagcaagcctgcctacgacagcgccgagtgccgccaccacgctgccgaggccctgcgtgtggcctggtgcctcgaagctgcctccctgtctcaccgacccggtcctcggtctggcctgtccgtgtccagcctgagacccaacagaaaaatggctacaaacttcctagcacatgagaagatctggttcgacaagttcaaatatgacgacgcagaaaggagattctacgagcagatgaacgggcctgtggcaggtgcctcccgccaggagaacggcgccagcgtgatcctccgtgacattgcgagagccagagagaacatccagaaatccctggctggaagctcaggccccggggcctccagcggcaccagcggagaccacggtgagctcgtcgtccggattgccagtctggaagtggagaaccagagtctgcgtggcgtggtacaggagctgcagcaggccatctccaagctggaggcccggctgaacgtgctggagaagagctcgcctggccaccgggccacggccccacagacccagcacgtatctcccatgcgccaagtggagcccccagccaagaagccagccacaccagcagaggatgacgaggatgatgacattgacctgtttggcagtgacaatgaggaggaggacaaggaggcggcacagctgcgggaggagcggctacggcagtacgcggagaagaaggccaagaagcctgcactggtggccaagtcctccatcctgctggatgtcaagccttgggatgatgagacggacatggcccagctggaggcctgtgtgcgctctatccagctggacgggctggtctggggggcttccaagctggtgcccgtgggctacggtatccggaagctacagattcagtgtgtggtggaggacgacaaggtggggacagacttgctggaggaggagatcaccaagtttgaggagcacgtgcagagtgtcgatatcgcagctttcaacaagatc

SEQ ID NO: 18 - DNA, 1407 bp, Escherichia coli
atggaggaaagaatggaaatgatttctgaaaggccaaaagagagtatgtattcctggaacaaaactgcagagaaaagtgattttgaagctgtagaagcacttatgtcaatgagctgcagttggaagtctgattttaagaaatacgttgaaaacagacctgttacaccagtatctgatttgtcagaggaagagaatctgcttccgggaacacctgattttcatacaatcccagcattttgtttgactccaccttacagtccttctgactttgaaccctctcaagtgtcaaatctgatggcaccagcgccatctactgtacacttcaagtcactctcagatactgccaaacctcacattgccgcacctttcaaagaggaagaaaagagcccagtatctgcccccaaactccccaaagctcaggcaacaagtgtgattcgtcatacagctgatgcccagctatgtaaccaccagacctgcccaatgaaagcagccagcatcctcaactatcagaacaattcttttagaagaagaacccacctaaatgttgaggctgcaagaaagaacataccatgtgccgctgtgtcaccaaacagatccaaatgtgagagaaacacagtggcagatgttgatgagaaagcaagtgctgcactttatgacttttctgtgccttcctcagagacggtcatctgcaggtctcagccagcccctgtgtccccacaacagaagtcagtgttggtctctccacctgcagtatctgcagggggagtgccacctatgccggtcatctgccagatggttccccttcctgccaacaaccctgttgtgacaacagtcgttcccagcactcctcccagccagccaccagccgtttgcccccctgttgtgttcatgggcacacaagtccccaaaggcgctgtcatgtttgtggtaccccagcccgttgtgcagagttcaaagcctccggtggtgagcccgaatggcaccagactctctcccattgcccctgctcctgggttttccccttcagcagcaaaagtcactcctcagattgattcatcaaggataaggagtcacatctgtagccacccaggatgtggcaagacatactttaaaagttcccatctgaaggcccacacgaggacgcacacaggagaaaagcctttcagctgtagctggaaaggttgtgaaaggaggtttgcccgttctgatgaactgtccagacacaggcgaacccacacgggtgagaagaaatttgcgtgccccatgtgtgaccggcggttcatgaggagtgaccatttgaccaagcatgcccggcgccatctatcagccaagaagctaccaaactggcagatggaagtgagcaagctaaatgacattgctctacctccaacccctgctcccacacag

SEQ ID NO: 19 - DNA, 2928 bp, Escherichia coli
atggaactgcaggctgctcgtgcttgcttcgctctgctgtggggttgcgctttggctgctgcagctgctgctcagggcaaagaggtggtcctgctggacttcgctgctgccggtggcgaactgggatggctgactcacccttacggcaagggctgggacctgatgcagaacatcatgaacgacatgcccatctacatgtactccgtgtgcaacgtgatgtccggcgaccaggacaactggctgcgtaccaactgggtgtaccgtggcgaggctgagcgcatcttcatcgagctgaagttcaccgtgcgcgactgcaactccttccctggtggtgcttccagctgcaaagagactttcaacctgtactacgctgagtccgacctggactacggcaccaacttccagaagcgtctgttcaccaagatcgacactatcgctcccgacgagatcaccgtgtcctccgacttcgaggctcgtcacgtgaagctgaacgtcgaggaacgctccgtgggtcccctgacccgcaagggattctacctggctttccaggacatcggtgcttgcgtggccctgctgtccgtgcgtgtgtactacaagaagtgccccgagctgctccagggcctggctcacttccctgagactatcgctggttccgacgctccctccctggctaccgttgctggtacttgcgtggaccacgctgtggtgccacctggtggcgaggaacctcgtatgcactgcgctgtggacggcgagtggctggtgcctatcggtcaatgcctgtgccaggctggttacgagaaggtcgaggacgcttgccaggcttgctcccccggtttcttcaagttcgaggcttccgagtccccctgcctggaatgccctgaacacaccctgccttccccagagggtgctacctcctgcgagtgcgaagagggcttcttccgtgctccccaggaccccgcttctatgccttgcacccgtcctccctccgctccccactacctgactgctgtcggcatgggtgctaaggtcgagctgcgttggaccccccctcaggattctggtggtcgcgaggacatcgtctactccgtgacctgcgagcagtgctggcctgagtctggcgaatgcggtccctgcgaggcttctgtgcgctactctgagcctcctcacggcctgacccgtacctctgtgaccgtgtccgacctcgagccccacatgaactacaccttcaccgtcgaggcccgtaacggtgtctccggactggtcacctcccgttccttccgtaccgcttccgtgtccatcaaccagaccgagccccccaaagtgcgcctggaaggacgttctaccacctccctgtccgtgtcttggtccatccccccacctcagcagtcccgtgtgtggaagtacgaagtgacctaccgcaagaagggcgactccaactcttacaacgtgcgtcgtaccgagggtttcagcgtgaccctggacgacctggctcccgacaccacctacctggtgcaagtgcaggctctgacccaagagggccagggtgctggttccaaggtgcacgagttccagaccctgtcccccgagggttccggaaacttggctgtgatcggcggtgtcgctgtgggtgtcgtgctgctgttggtgctggctggtgtcggcttcttcatccaccgtcgtcgcaagaaccagcgtgctcgtcagtcccctgaggacgtgtacttctccaagtccgagcagctgaagcccctcaagacctacgtggacccccacacttacgaggaccccaaccaggctgtgctcaagttcactaccgagatccacccctcctgcgtgacccgtcagaaagtgatcggtgctggcgagttcggcgaggtgtacaagggaatgctcaagacttcctccggcaagaaagaggtgcccgtcgctatcaagaccctgaaggctggctacaccgagaagcagcgtgtggacttcctgggagaggctggtatcatgggccagttctcccaccacaacatcatccgtctggaaggtgtcatctccaagtacaagcccatgatgatcattaccgagtacatggaaaacggcgccctggacaagttcctgcgcgagaaggatggcgagttctccgtgctgcagctcgtgggaatgctgcgtggtatcgctgctggcatgaagtacctggccaacatgaattacgtgcacagggacctggctgctcgcaacatcctggtcaactccaacctcgtgtgcaaggtgtcagacttcggcctgtcccgcgtgctcgaggacgatcctgaggctacctacaccacctccggtggaaagatccccatccgttggaccgctcccgaggctatctcttaccgcaagttcacctccgcttccgacgtgtggtccttcggtatcgtgatgtgggaagtgatgacctacggcgagcgcccctactgggagctgtctaaccacgaagtcatgaaggctatcaacgacggtttccgtctgcccacccctatggactgcccctccgctatctaccagctgatgatgcaatgctggcagcaagagcgtgctaggcgtcccaagttcgctgacatcgtgtctatcctcgacaagctgatccgcgctcctgactccctgaaaaccctggctgacttcgacccccgtgtgtccatccgcctgccttctacctctggctccgagggtgtccctttccgtactgtgtccgagtggctcgagtccatcaagatgcagcagtacaccgagcacttcatggctgctggttacaccgctatcgagaaggtggtgcagatgaccaacgacgacatcaagcgtatcggcgtgcgtctgcccggtcaccagaagaggatcgcttactccctgctgggcctgaaggaccaagtgaacaccgtgggtatccccatc

SEQ ID NO: 20 - DNA, 1143 bp, Escherichia coli
atggagtctggcagtaccgccgccagtgaggaggcacgcagccttcgagaatgtgagctctacgtccagaagcataacattcaagcgctgctcaaagattctattgtgcagttgtgcactgctcgacctgagagacccatggcattcctcagggaatactttgagaggttggagaaggaggaggcaaaacagattcagaatctgcagaaagcaggcactcgtacagactcaagggaggatgagatttctcctcctccacccaacccagtggttaaaggtaggaggcgacgaggtgctatcagcgctgaggtctacacggaggaagatgcggcatcctatgttagaaaggttataccaaaagattacaagacaatggccgctttagccaaagccattgaaaagaatgtgctgttttcacatcttgatgataatgagagaagtgatatttttgatgccatgttttcggtctcctttatcgcaggagagactgtgattcagcaaggtgatgaaggggataacttctatgtgattgatcaaggagagacggatgtctatgttaacaatgaatgggcaaccagtgttggggaaggagggagctttggagaacttgctttgatttatggaacaccgagagcagccactgtcaaagcaaagacaaatgtgaaattgtggggcatcgaccgagacagctatagaagaatcctcatgggaagcacactgagaaagcggaagatgtatgaggaattccttagtaaagtctctattttagagtctctggacaagtgggaacgtcttacggtagctgatgcattggaaccagtgcagtttgaagatgggcagaagattgtggtgcagggagaaccaggggatgagttcttcattattttagaggggtcagctgctgtgctacaacgtcggtcagaaaatgaagagtttgttgaagtgggaagattggggccttctgattattttggtgaaattgcactactgatgaatcgtcctcgtgctgccacagttgttgctcgtggccccttgaagtgcgttaagctggaccgacctagatttgaacgtgttcttggcccatgctcagacatcctcaaacgaaacatccagcagtacaacagttttgtgtcactgtctgtc

SEQ ID NO: 21 - DNA, 855 bp, Escherichia coli
atgaaccggcttccggatgactacgacccctacgcggttgaagagcctagcgacgaggagccggctttgagcagctctgaggatgaagtggatgtgcttttacatggaactcctgaccaaaaacgaaaactcatcagagaatgtcttaccggagaaagtgaatcatctagtgaagatgaatttgaaaaggagatggaagctgaattaaattctaccatgaaaacaatggaggacaagttatcctctctgggaactggatcttcctcaggaaatggaaaagttgcaacagctccgacaaggtactacgatgatatatattttgattctgattccgaggatgaagacagagcagtacaggtgaccaagaaaaaaaagaagaaacaacacaagattccaacaaatgacgaattactgtatgatcctgaaaaagataacagagatcaggcctgggttgatgcacagagaaggggttaccatggtttgggaccacagagatcacgtgaacaacagcctgttccaaatagtgatgctgtcttgaattgtcctgcctgcatgaccacactttgccttgattgccaaaggcatgaatcatacaaaactcaatatagagcaatgtttgtaatgaattgttctattaacaaagaggaggttctaagatataaagcctcagagaacaggaagaaaaggcgggtccataagaagatgaggtctaaccaggaagatgctgctgagaaggcagagacagatgtggaagaaatctatcacccagtcatgtgcactgaatgttccactgaagtggcagtctacgacaaggatgaagtctttcattttttcaatgttttagcaagccattcc

SEQ ID NO: 22 - protein, 408 aa, Escherichia coli
MAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNEERKQNKVEAKLRAKQEQEAKQKLEEDAEMKSLEEKIGCLLKFSGDLDDQTCREDLHILFSNHGEIKWIDFVRGAKEGIILFKEKAKEALGKAKDANNGNLQLRNKEVTWEVLEGEVEKEALKKIIEDQQESLNKWKSKGRRFKGKGKGNKAAQPGSGKGKVQFQGKKTKFASDDEHDEHDENGATGPVKRAREETDKEEPASKQQKTENGAGDQ

SEQ ID NO: 23 - protein, 538 aa, Escherichia coli
MEESVNQMQPLNEKQIANSQDGYVWQVTDMNRLHRFLCFGSEGGTYYIKEQKLGLENAEALIRLIEDGRGCEVIQEIKSFSQEGRTTKQEPMLFALAICSQCSDISTKQAAFKAVSEVCRIPTHLFTFIQFKKDLKESMKCGMWGRALRKAIADWYNEKGGMALALAVTKYKQRNGWSHKDLLRLSHLKPSSEGLAIVTKYITKGWKEVHELYKEKALSVETEKLLKYLEAVEKVKRTRDELEVIHLIEEHRLVREHLLTNHLKSKEVWKALLQEMPLTALLRNLGKMTANSVLEPGNSEVSLVCEKLCNEKLLKKARIHPFHILIALETYKTGHGLRGKLKWRPDEEILKALDAAFYKTFKTVEPTGKRFLLAVDVSASMNQRVLGSILNASTVAAAMCMVVTRTEKDSYVVAFSDEMVPCPVTTDMTLQQVLMAMSQIPAGGTDCSLPMIWAQKTNTPADVFIVFTDNETFAGGVHPAIALREYRKKMDIPAKLIVCGMTSNGFTIADPDDRGMLDMCGFDTGALDVIRNFTLDMI

SEQ ID NO: 24 - protein, 837 aa, Escherichia coli
MASKRKSTTPCMVRTSQVVEQDVPEEVDRAKEKGIGTPQPDVAKDSWAAELENSSKENEVIEVKSMGESQSKKLQGGYECKYCPYSTQNLNEFTEHVDMQHPNVILNPLYVCAECNFTTKKYDSLSDHNSKFHPGEANFKLKLIKRNNQTVLEQSIETTNHVVSITTSGPGTGDSDSGISVSKTPIMKPGKPKADAKKVPKKPEEITPENHVEGTARLVTDTAEILSRLGGVELLQDTLGHVMPSVQLPPNINLVPKVPVPLNTTKYNSALDTNATMINSFNKFPYPTQAELSWLTAASKHPEEHIRIWFATQRLKHGISWSPEEVEEARKKMFNGTIQSVPPTITVLPAQLAPTKVTQPILQTALPCQILGQTSLVLTQVTSGSTTVSCSPITLAVAGVTNHGQKRPLVTPQAAPEPKRPHIAQVPEPPPKVANPPLTPASDRKKTKEQIAHLKASFLQSQFPDDAEVYRLIEVTGLARSEIKKWFSDHRYRCQRGIVHITSESLAKDQLAIAASRHGRTYHAYPDFAPQKFKEKTQGQVKILEDSFLKSSFPTQAELDRLRVETKLSRREIDSWFSERRKLRDSMEQAVLDSMGSGKKGQDVGAPNGALSRLDQLSGAQLTSSLPSPSPAIAKSQEQVHLLRSTFARTQWPTPQEYDQLAAKTGLVRTEIVRWFKENRCLLKTGTVKWMEQYQHQPMADDHGYDAVARKATKPMAESPKNGGDVVPQYYKDPKKLCEEDLEKLVTRVKVGSEPAKDCLPAKPSEATSDRSEGSSRDGQGSDENEESSVVDYVEVTVGEEDAISDRSDSWSQAAAEGVSELAESDSDCVPAEAGQA

SEQ ID NO: 25 - protein, 441 aa, Escherichia coli
MEQPQEEAPEVREEEEKEEVAEAEGAPELNGGPQHALPSSSYTDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 26 - protein, 180 aa, Escherichia coli
MEQPTSSTNGEKRKSPCESNNKKNDEMQEAPNRVLAPKQSLQKTKTIEYLTIIVYYYRKHTKINSNQLEKDQSRENSINPVQEEEDEGLDSAEGSSQEDEDLDSSEGSSQEDEDLDSSEGSSQEDEDLDSSEGSSQEDEDLDSSEGSSQEDEDLDPPEGSSQEDEDLDSSEGSSQEGGED

SEQ ID NO: 27 - protein, 353 aa, Escherichia coli
MEKTLETVPLERKKREKEQFRKLFIGGLSFETTEESLRNYYEQWGKLTDCVVMRDPASKRSRGFGFVTFSSMAEVDAAMAARPHSIDGRVVEPKRAVAREESGKPGAHVTVKKLFVGGIKEDTEEHHLRDYFEEYGKIDTIEIITDRQSGKKRGFGFVTFDDHDPVDKIVLQKYHTINGHNAEVRKALSRQEMQEVQSSRSGRGGNFGFGDSRGGGGNFGPGPGSNFRGGSDGYGSGRGFGDGYNGYGGGPGGGNFGGSPGYGGGRGGYGGGGPGYGNQGGGYGGGYDNYGGGNYGSGNYNDFGNYNQQPSNYGPMKSGNFGGSRNMGGPYGGGNYGPGGSGGSGGYGGRSRY

SEQ ID NO: 28 - protein, 343 aa, Escherichia coli
MNIHRSTPITIARYGRSRNKTQDFEELSSIRSAEPSQSFSPNLGSPSPPETPNLSHCVSCIGKYLLLEPLEGDHVFRAVHLHSGEELVCKVFDISCYQESLAPCFCLSAHSNINQITEIILGETKAYVFFERSYGDMHSFVRTCKKLREEEAARLFYQIASAVAHCHDGGLVLRDLKLRKFIFKDEERTRVKLESLEDAYILRGDDDSLSDKHGCPAYVSPEILNTSGSYSGKAADVWSLGVMLYTMLVGRYPFHDIEPSSLFSKIRRGQFNIPETLSPKAKCLIRSILRREPSERLTSQEILDHPWFSTDFSVSNSAYGAKEVSDQLVPDVNMEENLDPFFN

SEQ ID NO: 29 - protein, 464 aa, Escherichia coli
MSSRSTKDLIKSKWGSKPSNSKSETTLEKLKGEIAHLKTSVDEITSGKGKLTDKERHRLLEKIRVLEAEKEKNAYQLTEKDKEIQRLRDQLKARYSTTTLLEQLEETTREGERREQVLKALSEEKDVLKQQLSAATSRIAELESKTNTLRLSQTVAPNCFNSSINNIHEMEIQLKDALEKNQQWLVYDQQREVYVKGLLAKIFELEKKTETAAHSLPQQTKKPESEGYLQEEKQKCYNDLLASAKKDLEVERQTITQLSFELSEFRRKYEETQKEVHNLNQLLYSQRRADVQHLEDDRHKTEKIQKLREENDIARGKLEEEKKRSEELLSQVQFLYTSLLKQQEEQTRVALLEQQMQACTLDFENEKLDRQHVQHQLHVILKELRKARNQITQLESLKQLHEFAITEPLVTFQGETENREKVAASPKSPTAALNESLVECPKCNIQYPATEHRDLLVHVEYCSK

SEQ ID NO: 30 - protein, 368 aa, Escherichia coli
MSVAGLKKQFYKASQLVSEKVGGAEGTKLDDDFKEMEKKVDVTSKAVTEVLARTIEYLQPNPASRAKLTMLNTVSKIRGQVKNPGYPQSEGLLGECMIRHGKELGGESNFGDALLDAGESMKRLAEVKDSLDIEVKQNFIDPLQNLCEKDLKEIQHHLKKLEGRRLDFDYKKKRQGKIPDEELRQALEKFEESKEVAETSMHNLLETDIEQVSQLSALVDAQLDYHRQAVQILDELAEKLKRRMREASSRPKREYKPKPREPFDLGEPEQSNGGFPCTTAPKIAASSSFRSSDKPIRTPSRSMPPLDQPSCKALYDFEPENDGELGFHEGDVITLTNQIDENWYEGMLDGQSGFFPLSYVEVLVPLPQ

SEQ ID NO: 31 - protein, 309 aa, Escherichia coli
MEQLLRAELRTATLRAFGGPGAGCISEGRAYDTDAGPVFVKVNRRTQARQMFEGEVASLEALRSTGLVRVPRPMKVIDLPGGGAAFVMEHLKMKSLSSQASKLGEQMADLHLYNQKLREKLKEEENTVGRRGEGAEPQYVDKFGFHTVTCCGFIPQVNEWQDDWPTFFARHRLQAQLDLIEKDYADREARELWSRLQVKIPDLFCGLEIVPALLHGDLWSGNVAEDDVGPIIYDPASFYGHSEFELAIALMFGGFPRSFFTAYHRKIPKAPGFDQRLLLYQLFNYLNHWNHFGREYRSPSLGTMRRLLK

SEQ ID NO: 32 - protein, 370 aa, Escherichia coli
MKIKDAKKPSFPWFGMDIGGTLVKLSYFEPIDITAEEEQEEVESLKSIRKYLTSNVAYGSTGIRDVHLELKDLTLFGRRGNLHFIRFPTQDLPTFIQMGRDKNFSTLQTVLCATGGGAYKFEKDFRTIGNLHLHKLDELDCLVKGLLYIDSVSFNGQAECYYFANASEPERCQKMPFNLDDPYPLLVVNIGSGVSILAVHSKDNYKRVTGTSLGGGTFLGLCSLLTGCESFEEALEMASKGDSTQADKLVRDIYGGDYERFGLPGWAVASSFGNMIYKEKRESVSKEDLARATLVTITNNIGSVARMCAVNEKINRVVFVGNFLRVNTLSMKLLAYALDYWSKGQLKALFLEHEGYFGAVGALLGLPNFS

SEQ ID NO: 33 - protein, 193 aa, Escherichia coli
MGKQNSKLRPEVLQDLRENTEFTDHELQEWYKGFLKDCPTGHLTVDEFKKIYANFFPYGDASKFAEHVFRTFDTNGDGTIDFREFIIALSVTSRGKLEQKLKWAFSMYDLDGNGYISRSEMLEIVQAIYKMVSSVMKMPEDESTPEKRTDKIFRQMDTNNDGKLSLEEFIRGAKSDPSIVRLLQCDPSSASQF

SEQ ID NO: 34 - protein, 490 aa, Escherichia coli
MEQKPSKVECGSDPEENSARSPDGKRKRKNGQCSLKTSMSGYIPSYLDKDEQCVVCGDKATGYHYRCITCEGCKGFFRRTIQKNLHPTYSCKYDSCCVIDKITRNQCQLCRFKKCIAVGMAMDLVLDDSKRVAKRKLIEQNRERRRKEEMIRSLQQRPEPTPEEWDLIHIATEAHRSTNAQGSHWKQRRKFLPDDIGQSPIVSMPDGDKVDLEAFSEFTKIITPAITRVVDFAKKLPMFSELPCEDQIILLKGCCMEIMSLRAAVRYDPESDTLTLSGEMAVKREQLKNGGLGVVSDAIFELGKSLSAFNLDDTEVALLQAVLLMSTDRSGLLCVDKIEKSQEAYLLAFEHYVNHRKHNIPHFWPKLLMKEREVQSSILYKGAAAEGRPGGSLGVHPEGQQLLGMHVVQGPQVRQLEQQLGEAGSLQGPVLQHQSPKSPQQRLLELLHRSGILHARAVCGEDDSSEADSPSSSEEEPEVCEDLAGNAASP

SEQ ID NO: 35 - protein, 613 aa, Escherichia coli
MFRCGGLAAGALKQKLVPLVRTVCVRSPRQRNRLPGNLFQRWHVPLELQMTRQMASSGASGGKIDNSVLVLIVGLSTVGAGAYAYKTMKEDEKRYNERISGLGLTPEQKQKKAALSASEGEEVPQDKAPSHVPFLLIGGGTAAFAAARSIRARDPGARVLIVSEDPELPYMRPPLSKELWFSDDPNVTKTLRFKQWNGKERSIYFQPPSFYVSAQDLPHIENGGVAVLTGKKVVQLDVRDNMVKLNDGSQITYEKCLIATGGTPRSLSAIDRAGAEVKSRTTLFRKIGDFRSLEKISREVKSITIIGGGFLGSELACALGRKARALGTEVIQLFPEKGNMGKILPEYLSNWTMEKVRREGVKVMPNAIVQSVGVSSGKLLIKLKDGRKVETDHIVAAVGLEPNVELAKTGGLEIDSDFGGFRVNAELQARSNIWVAGDAACFYDIKLGRRRVEHHDHAVVSGRLAGENMTGAAKPYWHQSMFWSDLGPDVGYEAIGLVDSSLPTVGVFAKATAQDNPKSATEQSGTGIRSESETESEASEITIPPSTPAVPQAPVQGEDYGKGVIFYLRDKVVVGIVLWNIFNRMPIARKIIKDGEQHEDLNEVAKLFNIHED

SEQ ID NO: 36 - protein, 461 aa, Escherichia coli
MNNFGNEEFDCHFLDEGFTAKDILDQKINEVSSSDDKDAFYVADLGDILKKHLRWLKALPRVTPFYAVKCNDSKAIVKTLAATGTGFDCASKTEIQLVQSLGVPPERIIYANPCKQVSQIKYAANNGVQMMTFDSEVELMKVARAHPKAKLVLRIATDDSKAVCRLSVKFGATLRTSRLLLERAKELNIDVVGVSFHVGSGCTDPETFVQAISDARCVFDMGAEVGFSMYLLDIGGGFPGSEDVKLKFEEITGVINPALDKYFPSDSGVRIIAEPGRYYVASAFTLAVNIIAKKIVLKEQTGSDDEDESSEQTFMYYVNDGVYGSFNCILYDHAHVKPLLQKRPKPDEKYYSSSIWGPTCDGLDRIVERCDLPEMHVGDWMLFENMGAYTVAAASTFNGFQRPTIYYVMSGPAWQLMQQFQNPDFPPEVEEQDASTLPVSCAWESGMKRHRAACASASINV

SEQ ID NO: 37 - protein, 772 aa, Escherichia coli
MGDEDDDESCAVELRITEANLTGHEEKVSVENFELLKVLGTGAYGKVFLVRKAGGHDAGKLYAMKVLRKAALVQRAKTQEHTRTERSVLELVRQAPFLVTLHYAFQTDAKLHLILDYVSGGEMFTHLYQRQYFKEAEVRVYGGEIVLALEHLHKLGIIYRDLKLENVLLDSEGHIVLTDFGLSKEFLTEEKERTFSFCGTIEYMAPEIIRSKTGHGKAVDWWSLGILLFELLTGASPFTLEGERNTQAEVSRRILKCSPPFPPRIGPVAQDLLQRLLCKDPKKRLGAGPQGAQEVRNHPFFQGLDWVALAARKIPAPFRPQIRSELDVGNFAEEFTRLEPVYSPPGSPPPGDPRIFQGYSFVAPSILFDHNNAVMTDGLEAPGAGDRPGRAAVARSAMMQDSPFFQQYELDLREPALGQGSFSVCRRCRQRQSGQEFAVKILSRRLEANTQREVAALRLCQSHPNVVNLHEVHHDQLHTYLVLELLRGGELLEHIRKKRHFSESEASQILRSLVSAVSFMHEEAGVVHRDLKPENILYADDTPGAPVKIIDFGFARLRPQSPGVPMQTPCFTLQYAAPELLAQQGYDESCDLWSLGVILYMMLSGQVPFQGASGQGGQSQAAEIMCKIREGRFSLDGEAWQGVSEEAKELVRGLLTVDPAKRLKLEGLRGSSWLQDGSARSSPPLRTPDVLESSGPAVRSGLNATFMAFNRGKREGFFLKSVENAPLAKRRKQKLRSATASRRGSPAPANPGRAPVASKGAPRRANGPLPPS

SEQ ID NO: 38 - protein, 281 aa, Escherichia coli
MATNFLAHEKIWFDKFKYDDAERRFYEQMNGPVAGASRQENGASVILRDIARARENIQKSLAGSSGPGASSGTSGDHGELVVRIASLEVENQSLRGVVQELQQAISKLEARLNVLEKSSPGHRATAPQTQHVSPMRQVEPPAKKPATPAEDDEDDDIDLFGSDNEEEDKEAAQLREERLRQYAEKKAKKPALVAKSSILLDVKPWDDETDMAQLEACVRSIQLDGLVWGASKLVPVGYGIRKLQIQCVVEDDKVGTDLLEEEITKFEEHVQSVDIAAFNKI

SEQ ID NO: 39 - protein, 480 aa, Escherichia coli
MLNFGASLQQTAEERMEMISERPKESMYSWNKTAEKSDFEAVEALMSMSCSWKSDFKKYVENRPVTPVSDLSEEENLLPGTPDFHTIPAFCLTPPYSPSDFEPSQVSNLMAPAPSTVHFKSLSDTAKPHIAAPFKEEEKSPVSAPKLPKAQATSVIRHTADAQLCNHQTCPMKAASILNYQNNSFRRRTHLNVEAARKNIPCAAVSPNRSKCERNTVADVDEKASAALYDFSVPSSETVICRSQPAPVSPQQKSVLVSPPAVSAGGVPPMPVICQMVPLPANNPVVTTVVPSTPPSQPPAVCPPVVFMGTQVPKGAVMFVVPQPVVQSSKPPVVSPNGTRLSPIAPAPGFSPSAAKVTPQIDSSRIRSHICSHPGCGKTYFKSSHLKAHTRTHTGEKPFSCSWKGCERRFARSDELSRHRRTHTGEKKFACPMCDRRFMRSDHLTKHARRHLSAKKLPNWQMEVSKLNDIALPPTPAPTQ

SEQ ID NO: 40 - protein, 976 aa, Escherichia coli
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 41 - protein, 381 aa, Escherichia coli
MESGSTAASEEARSLRECELYVQKHNIQALLKDSIVQLCTARPERPMAFLREYFERLEKEEAKQIQNLQKAGTRTDSREDEISPPPPNPVVKGRRRRGAISAEVYTEEDAASYVRKVIPKDYKTMAALAKAIEKNVLFSHLDDNERSDIFDAMFSVSFIAGETVIQQGDEGDNFYVIDQGETDVYVNNEWATSVGEGGSFGELALIYGTPRAATVKAKTNVKLWGIDRDSYRRILMGSTLRKRKMYEEFLSKVSILESLDKWERLTVADALEPVQFEDGQKIVVQGEPGDEFFIILEGSAAVLQRRSENEEFVEVGRLGPSDYFGEIALLMNRPRAATVVARGPLKCVKLDRPRFERVLGPCSDILKRNIQQYNSFVSLSV

SEQ ID NO: 42 - protein, 285 aa, Escherichia coli
MNRLPDDYDPYAVEEPSDEEPALSSSEDEVDVLLHGTPDQKRKLIRECLTGESESSSEDEFEKEMEAELNSTMKTMEDKLSSLGTGSSSGNGKVATAPTRYYDDIYFDSDSEDEDRAVQVTKKKKKKQHKIPTNDELLYDPEKDNRDQAWVDAQRRGYHGLGPQRSRQQQPVPNSDAVLNCPACMTTLCLDCQRHESYKTQYRAMFVMNCSINKEEVLRYKASENRKKRRVHKKMRSNREDAAEKAETDVEEIYHPVMCTECSTEVAVYDKDEVFHFFNVLASHS